(19) United States
(12) Patent Application Publication
(10) Pub. No.: US 2006/0063727 A1
(43) Pub. Date: Mar. 23, 2006

(54) Tr4/tr2 response elements

(76) Inventor: Chawnshang Chang, Pittford, NY (US)

Correspondence Address: NEEDLE & ROSENBERG, P.C., SUITE 1000, 999 PEACHTREE STREET, ATLANTA, GA, 30309-3915, US

(21) Appl. No.: 10/514,581

(22) PCT Filed: May 19, 2003
(86) PCT No.: PCT/US03/15926

§ 371 Date: Jan. 18, 2005

Related U.S. Application Data
(60) Provisional application No. 60/381,438, filed on May 17, 2002.

(51) Int. Cl. A61K 48/00; C12Q 1/70; C12Q 1/68

(52) U.S. Cl. 514/44; 435/5; 435/6

(57) ABSTRACT

Disclosed are compositions and methods related to TR2 and TR4.

This application claims benefit of U.S. Provisional Application No. 60/381,438, filed May 17, 2002, which is hereby incorporated herein by reference in its entirety.

### I. ACKNOWLEDGEMENTS

This work was supported by NIH grants, DK47258, DK1346, and DK56884, and the United States Government may have certain rights in this invention.

### II. BACKGROUND OF THE INVENTION

Orphan receptors, or receptors without known ligands, make up the vast majority of members of the steroid receptor superfamily (Evans, R. M. (1988) Science 240, 889-895; Chang, C., and Kokontis, J. (1988) Biochem. Biophys. Res. Commun. 155, 971-977; Chang, C. et al. (1989) Biochem. Biophys. Res. Commun. 165, 735-741). Members of this family are characterized by a highly conserved DNA-binding domain (DBD) and a carboxy-terminal ligand-binding domain. The conserved amino acid sequence within the DBDs of these receptors is predicted to form two zinc finger motifs (Evans, R. M. (1988) Science 240, 889-895). Additionally, steroid receptors bind to specific DNA sequences known as hormone response elements (HREs), through which regulation of target gene expression may occur (Evans, R. M. (1988) Science 240, 889-895). This orphan receptor binds to a consensus HRE composed of a direct repeat (DR) with variable spacing (AGGTCAX(n)AGGTCA, n=0-6) (Lin, T. M. et al. (1995) J. Biol. Chem. 270, 30121-30128). Using the known TR2 HRE sequence as a guide, it was determined that TR2 has modulatory effects on several signaling pathways, such as those involving retinoic acid (Lin, T. M. et al. (1995) J. Biol. Chem. 270, 30121-30128), thyroid hormone (Chang, C., and Pan, H. (1998) Mol. Cel. Biochem. 189, 195-200), and ciliary neurotrophic factor (Young, W. et al. (1998) J Biol. Chem. 273, 20877-20885). Moreover, expression of the erythropoietin gene (Lee, H. J., and Chang, C. (1995) J. Biol. Chem. 270, 5434-5440), aldolase gene (Chang, C., and Lee, H. (1997) Biochem. Biophys. Res. Commun. 235, 205-211), histamine H1 receptor (Lee, H., and Chang, C. (1999) Mol. Cel. Biochem. 194, 199-207), and Simian Virus 40 (SV40) (Lee, H. J. et al. (1996) J. Biol. Chem. 271, 10405-10412) is also regulated by TR2.

### III. SUMMARY OF THE INVENTION

In accordance with the purposes of this invention, as embodied and broadly described herein, this invention, in one aspect, relates to compositions and method related to TR2 and TR4 DNA binding.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### IV. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1. shows that TR2 protein is expressed at potential site of HPV-16 infection. Saggital sections of the vagina/cervix from adult female ICR mice (Taconic) were stained with hematoxylin/eosin (FIG. 1A), using phosphate-buffered saline in place of primary antibody (FIG. 1B), or with an anti-TR2 monoclonal antibody (FIG. 1C). Prominent TR2 staining is observed in nuclei of cells in the basal layers of the stratified squamous epithelium (FIG. 1E) of the mouse vagina. LP, lamina propria; SM, smooth muscle.

FIG. 2 shows that the HPV-16 oncoprotein E6 antagonizes p53-mediated repression of TR2. Transient transfections were performed in the H1299 cell line using the TR2-promoter linked to the CAT gene (TR2pCAT) as a reporter. TR2pCAT was co-transfected with constructs expressing either wildtype p53, full length E6 and E7 (p1321), or E6 and E7 with an early termination codon in the E6 open reading frame (p1434). In the histogram, CAT activity produced under each condition is displayed relative to that produced with the reporter alone (lanes 2-5 vs. lane 1). Expression of p53 in the p53-null H1299 cell line resulted in suppression of TR2 promoter-linked reporter expression (lane 2). Addition of a construct expressing E6 and E7 eliminated the repression of TR2 mediated by p53 (lanes 3-4), while a truncated version of E6 was unable to reverse the suppressive effect of p53 (lane 5).

FIG. 3 shows the functional role of TR2 relative to HPV-16 LCR-directed gene expression. Gel-shift analysis was performed using in vitro expressed TR2 protein and 32 P-labeled HPV-16-DR4RE. FIG. 3A. Binding reaction mixture containing the isotope-labeled probe was incubated with TR2 alone (lane 1), or in the presence of either excess unlabeled oligonucleotide (10×) (lane 2), or mock translated TR2 protein (lane 3). Lane 4 contains only the 32 P-labeled probe. The complex between TR2 and the isotope-labeled HPV sequence is indicated by the arrow (lane 1). FIG. 3B. Lane 1 contains the 32 P-labeled probe alone. The 32 P-labeled HPV-16 DR4RE probe was incubated with mock translated TR2 (lane 2), in vitro synthesized TR2 (lane 4), or TR2 along with a monoclonal anti-TR2 antibody Cane 3). TR2 in complex with HPV-16 DR4RE is indicated by arrow II (lane 4), and the antibody-supershifted complex is indicated by arrow I (lane 3)

FIG. 4 shows the TR2-mediated induction of HPV-16 gene expression. Transient transfection assays were performed in CHO cells, using an HPV-16 long control region (LCR)-derived CAT reporter. Levels of CAT activity upon co-transfection of increasing doses of either pSG5-hTR2-11 (TR2) (lanes 2-5), pSG5-ARp-TR2 (2A2) (lanes 6-9), or pCMX-TR4 (TR4) (lanes 10-13) are displayed relative to the CAT activity of the reporter alone (lane 1). The chimeric TR2 receptor, lacking the TR2-specific P-box which is important for DNA binding, is unable to induce HPV-16 reporter gene expression.

FIG. 5 shows the functional analysis of HPV-16 DR4RE. FIG. 5A. A portion of the HPV-16 LCR sequence, containing the TR2-responsive HPV-DR4RE, is shown. The DR4RE contains two A(G/T)(G/T)TCA half-sites located 175 base pairs upstream of the TATA-box at the start of the E6 ORF. FIG. 5B. The HPV-16 LCR-D27CAT mutant plasmid was constructed via site-directed mutagenesis PCR. A 27 bp region, containing the HPV-16-DR4RE, was deleted from the HPV-16 LCR-CAT construct. C. The HPV-16 LCR-D27CAT mutant reporter was co-transfected with plasmids expressing either orphan receptor TR2 (pSG5-hTR2-11) or the highly homologous orphan receptor, TR4 (pCMXTR4). Neither TR2 (lanes 2-4), nor TR4 (lanes 5-7) was able to induce expression of the reporter, as the receptor binding site (DR4RE) is no longer present in the mutant construct.

FIG. 6 shows that HPV-DR4RE upregulates transcriptional activity of the TK minimal promoter in CHO cells. FIG. 6A. The 16 bp HPV-16 DR4RE oligonucleotide (bold arrow) was inserted into the 32TK-CAT vector in either sense (+) or antisense (−) orientation. FIG. 6B. The reporter gene expression of both constructs is significantly up-regulated upon addition of increasing amounts of either TR2 (pSG5-hTR2-1 1) or TR4 (pCMX-TR4).

FIG. 7 shows the feedback regulation between TR2 and HPV-16. This figure is a schematic representation of the regulatory pathway involving both the TR2 orphan receptor and HPV-16, as suggested by our study. We hypothesize that, through the binding of TR2 molecules at the DR4 response element sequence in the upstream long control region of HPV-16, TR2 is able to increase expression of the HPV-16 early gene product, E6. From here, E6 is involved in promoting the ubiquitin-mediated degradation of tumor suppressor p53. As p53 exhibits a repressive effect on TR2 expression, enhanced E6 expression and the resulting enhancement of p53 degradation may allow an increase in TR2 protein level.

FIG. 8 shows the binding preference of TR4 to different DRs. FIG. 8 shows a Sequence List of Two Direct-Repeats and Monomer DNA Response Elements. The core consensus AGGTCA sequences (in capitals) of direct repeats (A) or monomeric (13) response elements are aligned. FIG. 8C, binding Affinity of the TR4 to Direct Repeats of Synthetic Response Elements. (a) Competitive EMSA. Six tandem repeats of the consensus AGGTCA sequence spaced by 1 to 6 bp (DR1-6) were serially diluted from 1.6 to 0.00625 ng to compete with 0.1 ng 32P-labeled DRs for binding to the in vitro translated TR4 (1 □1) in EMSA. (b) RBA values. The RBA was determined as a percentage of the radioactivity of the DNA-protein complex obtained with the lowest concentration cold competition. IC50 was calculated from the curves shown in (a) FIG. 8D, binding Affinity of the TR4 to Direct Repeats of Natural Response Elements. (a) Competitive EMSA. CRBPII, SV40+55, VDRE, TRE, RARE, and ERE all contain direct repeats of the AGGTCA core sequence, and were tested in the same conditions as in FIG. 1B (b) RBA values.

FIG. 9 shows the binding of the In Vitro expressed TR4 to the monomeric response elements. FIG. 9(A) Binding of the TR4 to the NBRE. EMSA was performed with the in vitro expressed TR4 and the ³²P-labeled NBRE probe. Lane 1 displays the probe alone, which contains the 30-bp NBRE sequence (29, 30). Binding reaction mixtures were incubated with the probe and either the mock-translated product (lane 2), or the in vitro synthesized TR4 (lanes 3-7) in the presence of 50 fold molar excesses of unlabeled NBRE (lane 4), preimmune serum (preim, lane 5), monoclonal anti-TR4 antibody (G232-416.3, lane 6), or monoclonal anti-TR4 antibody (G232-85.6, lane 7). The retarded complexes are indicated by the arrowhead for specific DNA-protein complexes, whereas the supershift band is marked by the arrow for DNA-protein-antibody complexes. Nonspecific complexes appear between the retarded complexes and the free probe at the bottom. FIG. 9(B) Binding Affinity of the TR4 to the Monomeric NBRE. Binding of the TR4 to different concentrations of the NBRE probe in EMSA. Constant amounts of the in vitro expressed TR4 (1 μl) were incubated with various amounts of the probe (0.05-6.4 ng). The specific protein-DNA complex (arrowhead) and the free probe at the bottom were quantified by PhosphorImager (Molecular Dynamics). FIG. 9(C) Scatchard analysis for the NBRE probe. The ratio between specific DNA-protein binding (bound, nM) and free DNA probe with respect to specific DNA-protein binding (bound/free) was plotted. The dissociation constant (K_{d}) and Bₘₐₓ values were generated from the Ebda program (Biosoft).

FIG. 10 shows that TR4 represses 21-OHase gene expression via the -228TR4RE by Dual-luciferase Reporter Assay. Mouse Y-1 cells were co-transfected with the pCMX-TR4 expression plasmid (lanes 2 and 4) and either the parent pGL3-promoter (lanes 1 and 2) or pGL3-228 (lanes 3 and 4) reporter plasmid. All firefly luciferase activities were normalized with Renilla luciferase activities, and then averaged over at least three independent experiments with error bars designating standard deviations. Significant (p<0.05) difference from control is marked with an asterisk.

FIG. 11 shows the sequence comparison of a regulatory region among 21-OHase Genes. The nucleotide sequences of the homologous regions of three 21-OHase genes are shown (43). Different species of the 21-OHase genes are human (GenBank™ accession no. M12792 and M23280), mouse (M15009 and M64933), and bovine (M11267 and M13545), respectively. Identical bases are indicated by a hyphen. The monomeric response element is boxed.

FIG. 12 shows a higher expression level of TR4 in S-phase in P19 cells. FIG. 12A, 1-2×10⁸ P19 cells were centrifuged and loaded into the Sanderson separation chamber. About 0.5-1×10⁶ cells from each fraction were fixed for DNA analysis on an Epics Profile flow cytometer. The cell cycle stage from five populations (G1, G1/S, S, early G2, and G2M) and unseparated (L) were determined and represented in DNA histograms. Panel B. Total RNA (25 μg) was isolated from each cell fraction. The probe used for the hybridization was the N-terminal domain of TR4 and was labeled randomly with α[³²P]dCTP. The hybridization bands were quantified by Phospolmager. Positions of 28S and 18S are indicated as well as the location of β-actin and TR4 transcripts.

FIG. 13 shows a strategy for establishing the P19 stable transfectant expressing anti-sense TR4 (P19αTR4) under control of a tetracycline-inducible system. FIG. 13A, schematic representation of the main features of two expression vectors, pCMV-tetR-KRAB-hyg and ptetO₇-CMV-αTR4-neo. FIG. 13B, the protocol for obtaining the P19αTR4. Five μg of these two expression vectors were co-transfected into the mouse teratocarcinoma P19 cells by the lipofectin method and then selected with Hgy B (300 μg/ml) and G418 (500 μg/ml). Single colonies were obtained by a ½ to ½¹² serial dilution after two to three months.

FIG. 14 shows a northern blot analysis of neo expression in P19αTR4 clones. Twenty-five μg of total RNA isolated from clones #1, 2, 4, and 5 of P19αTR4 (lanes 1 to 4) or P19 parental cells (lane 5). The probe used for the hybridization was neo, random labeled with α[³²P]-dCTP.

FIG. 15 shows the inhibition of TR4 expression in P19αTR4 by adding doxycycline. P19αTR4 and parental P19 cells were treated with or without 1 αg/ml doxycycline for 48 h. Three μg nuclear extracts from P19αTR4 (lanes 4, 5, 9, and 10) or P19 cells (lanes 2, 3, 7, and 8) were incubated with 0.1 ng of [³²P]-CNTFR-DR1. As a control, 1 μl of in vitro translated TR4 was also added (lanes 1, and 6). In order to distinguish TR4 from other DR1-binding proteins, 1 μl of anti-TR4 antibody was added to the reaction (lanes 6-10). The reaction mixtures were separated on 5% non-denaturing gel. The DNA-TR4, and DNA-TR4-McAb complexes were indicated as an arrowhead and arrow, respectively.

FIG. 16 shows the morphological changes of P19αTR4 cells during the RA-treatment in the presence or absence of doxycycline. The P19αTR4 cells were either treated (FIG. 16A, C, and E) or no-treated (FIG. 16B, D, and F) with doxycycline for 48 h. The cells were then treated with 10⁻⁶ M atRA for 24 h (A and B), 48 h (FIG. 16C and D) and 72 h (FIG. 16E and F) and observed under a microscope. Pictures were taken under 10×10 magnification.

FIG. 17 shows a DNA ladder formation in RA-treated P19αTR4 cells. The P19αTR4 cells were treated with atRA for 72 h with or without addition of doxycycline. The cells were harvested and DNA fragmentation analysis was performed. Lane 1 represents the 1 kb standard marker.

FIG. 18 shows that TR2 protein is expressed in HaCaT keratinocytes. Twenty μg of cell lysate from subconfluent HaCaT keratinocytes was subjected to SDS-PAGE and Western blotting, followed by immunoblotting with the monoclonal antibody against TR2. Bars on the left margin represent the molecular mass marker in kilodaltons.

FIG. 19 shows that TR2 protein is increased during keratinocyte differentiation. At 0, 24, 48, and 72 hours after incubating in 1.8 mM Ca²⁺, normal human keratinocytes were harvested. Twenty ,g of cell lysates were subjected to Western blot analysis. The upper graph is the quantification of each band in the lower immunoblot.

FIG. 20 shows that TR2 can suppress RA-induced pRARβ-CAT, but not vitamin D-induced P450cc24-CAT reporter activity. The reporter plasmids (4 μg of pRARβ-CAT and P450cc24-CAT) were transfected into HaCaT keratinocytes in the presence of the indicated amount of pCMV-TR2 plasmid. The cells were treated with 10⁻⁶ M all-trans retinoic acid, 10⁻⁷ M 1,25—(OH)₂D₃, or ethanol only, as indicated. CAT activities were determined and expressed as relative activity of each reporter in the presence of all-trans retinoic acid or 1,25—(OH)₂D₃ but in the absence of pCMV-TR2 plasmid. Error bars represent the mean±S.D. from three independent experiments. *Significant difference from the control (lane 2), p<0.01.

FIG. 21 shows TR2 can specifically bind to PPRE. In vitro translated TR2 (1 μl) was incubated with 0.1 ng ³²P-labeled PPRE oligomer and analyzed by an electrophoretic mobility shift assay. Lane 1: labeled probe alone; lane 2: the probe and the mock translated control; lane 3: the probe and the in vitro translated TR2 protein; lane 4: the probe, the TR2 protein, and a 100-fold molar excess of PPRE; lane 5: the probe, the TR2 protein, and anti-TR2 serum; lane 6: the probe, the TR2 protein, and preimmune serum.

FIG. 22 shows that TR2 and TR4 differentially suppress PPAR transactivation in HaCaT keratinocytes. The reporter plasmids (4 μg of PPRE₃-tk-LUC) were transfected into HaCaT keratinocytes in the presence of various amounts (lanes 1 and 2: 0 μg; lanes 3 and 8: 0.01 μg; lanes 4 and 9: 0.25 μg; lanes 5 and 10: 0.5 μg; lanes 6 and 11: 0.75 μg; lanes 7 and 12: 1 μg) of pCMV-TR2 or pCMX-TR4. The cells were treated with 10⁻⁵ M WY-14643 or ethanol only, as indicated. Luciferase activities were determined and expressed as relative activity of the reporter in the presence of Wy-14643 but in the absence of pCMV-TR2 and pCMX-TR4. Error bars represent mean±S.D. from three independent experiments. *Significant difference from the control (lane 2), p<0.01.

FIG. 23 shows that the affinity of TR2 for binding to PPRE is weaker than that of TR4. Scatchard analysis of the binding of the ³²P-labeled probe (PPRE) to in vitro translated orphan receptors TR2 (a) and TR4 (b). Band shifts were developed and quantified by ImageQuant software. To quantify the total probe in the reaction, as known amount of the probe was spotted onto a filter paper and then quantified by ImageQuant software.

FIG. 24 shows that the Wy-14643 upregulates the expression level of TR4 protein and decreases that of TR2 protein. At 0, 24, 48 and 72 hours after the addition of 10⁻⁵ M Wy-14643, HaCaT keratinocytes were harvested. Forty μg of cell lysates were subjected to Western blot analysis for TR4 (a) or TR2 (b). Each upper graph is the quantification of each band in the lower immunoblot.

### V. DETAILED DESCRIPTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Abbreviations: CAT, chloramphenicol acetyltransferase; DBD, DNA-binding domain; E2, 17β-estradiol; ER, estrogen receptor; ERE, estrogen response element; GST, glutathione S-transferase; LBD, ligand-binding domain; PR, progesterone receptor; TR2, TR2 human testicular orphan receptor 2, TR4, TR4 human testicular orphan receptor 4; RA, retinoic acid; PPARα peroxisome proliferator-activated receptor α; CAT, chloramphenicol acetyltransferase; RAR, retinoic acid receptor; PPRE, peroxisome proliferator response element; 1,25—(OH)2D3, 1,25-dihydroxyvitamin D3; Kd, equilibrium dissociation constant, HRE, hormone receptor response element; DR, direct repeat, HPV-16, human papilloma virus type 16; LCR, long control region; CAT, chloramphenicol acetyltransferase; DBD, DNA binding domain; CHO, chinese hamster ovary; TK, thymidine kinase.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular TR4 or TR2 is disclosed and discussed and a number of modifications that can be made to a number of molecules including the TR4 or TR2 are discussed, specifically contemplated is each and every combination and permutation of TR4 and TR2 and the modifications that are possible unless specifically indicated to the contrary. Thus, if a class of molecules A, B. and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

B. Compositions and Methods

The human TR4 orphan receptor (TR4) is a member of the steroid/thyroid hormone receptor superfamily. It has been documented that the TR4 may bind as a homodimer to a DNA response element containing two direct repeats of the AGGTCA consensus motif However, the data disclosed herein indicate that the expression of the human steroid 21-hydroxylase (21-OHase) gene could be repressed by the TR4 via the monomeric AGGTCA motif (−228TR4RE) at its 5′ flanking region (nucleotide numbers 1431-1444, 5′-GGAAAAAGGTCAGG-3′). Electrophoretic mobility shift assay showed specific binding with dissociation constants (K_{d}) of 0.2 nM between the TR4 and the monomeric −288TR4RE motif. Additionally, dual-luciferase assay demonstrated that the TR4 can function as a repressor via the −228TR4RE of the 21-OHase gene. Thus the disclosed data indicate that the TR4 orphan receptor may bind to a monomeric DNA response element and play an important role in the suppression of 21-OHase gene expression.

Disclosed herein, a TR2 DR4 consensus site within the long control region (LCR) of the human papilloma virus type 16 (HPV-16) has been found. Also disclosed herein it is shown that TR2 is able to bind the consensus response element (DR4RE) within the HPV-16 gene, and modulate HPV-16 expression.

Also disclosed herein TR4 alters the retinoic acid-induced differentiation pathways that result in the changes of cell morphology and cell cycle profile, including apoptosis. Also disclosed, this TR4 effect can be altered by the production of antisense-TR4.

Steroid 21-hydroxylase (21-OHase; EC 1.14.99.10) belongs to the cytochrome P-450 superfamily, and is one of the key enzymes in biosynthesis of the steroid hormones, leading to the production of cortisol and aldosterone (Miller, W. L. (1988) Endocrine Rev. 9, 295-318). Disclosed herein, the 21-Ohase gene contains a perfect monomeric DNA response element containing the single AGGTCA core motif preceded by an AT-rich sequence located in the −228 element in the human 21-OHase gene (White, P. C., et al. (1986) Proc. Natl. Acad. Sci. USA 83, 5111-5115), and that the TR4 can function as a repressor in human steroid 21-hydroxylase gene regulation via the −228TR4RE.

Also disclosed herein, are interactions of TR2 and TR4 in the (peroxisome proliferator activated receptor alpha) PPARα signal pathway.

Disclosed are methods of inhibiting HPV infection comprising interfering with TR2 or TR4 interaction with the TR2/TR4 response element in the HPV genome.

Also disclosed are methods, wherein the interfering occurs in stratified squamous epithelial cells, wherein the interfering occurs in vaginal or cervix tissue, wherein the response element is a DR4 response element, wherein the DR4 response element is located 175 bp upstream of the TATA-box at bp 65 of the E6 ORF, wherein the consensus DR response element comprises the sequence 5′-A(G/T)(G/T)TCA)-3′, wherein the DR4 response element comprises the sequence 5′-A(G/T)(G/T)TCAccctA(G/T)(G/TCA)-3′, and/or wherein the DR4 response element comprises the sequence 5′ATGTCAccctAGTTCA-3′.

Also disclosed are methods of identifying an inhibitor of TR2 or TR4 HPV DR4 binding comprising forming an incubation mixture comprising a potential inhibitor, TR2, TR4, or combination thereof, and a nucleic acid comprising 5′-A(G/T)(G/T)TCA)-3′, 5′-A(G/T)(G/T)TCAccctA(G/T)(G/T)TCA)-3′, or 5′ATGTCAccctAGTTCA-3′ in double stranded form, and assaying whether the potential inhibitor decreases the amount of TR2, TR4, or combination thereof bound to 5′-A(G/T)(G/T)TCA)-3′, 5′-A(G/T)(G/T)TCAccctA(G/T)(G/T)TCA)-3′, or 5′ATGTCAccctAGTTCA-3′.

Disclosed are methods of producing an inhibitor of TR2 or TR4 HPV DR4 binding comprising identifying a potential inhibitor of TR2 or TR4 HPV DR4 binding comprising forming an incubation mixture comprising a potential inhibitor, TR2, TR4, or combination thereof, and a nucleic acid comprising 5′-A(G/T)(G/T)TCA)-3′, 5′-A(G/T)(G/T)TCAccctA(G/T)(G/T)TCA)-3′, or 5′ATGTCAccctAGTTCA-3′ in double stranded form, and assaying whether the potential inhibitor decreases the amount of TR2, TR4, or combination thereof bound to 5′-A(G/T)(G/T)TCA)-3′, 5′-A(G/T)(G/T)TCAccctA(G/T)(G/T)TCA)-3′, or 5 ′ATGTCAccctAGTTCA-3′.

Also disclosed are methods of manufacturing an inhibitor of TR2 or TR4 HPV DR4 binding comprising synthesizing the potential inhibitor disclosed herein.

Also disclosed are methods of manufacturing a potential inhibitor of TR2 or TR4 HPV DR4 binding comprising admixing the potential inhibitor of disclosed herein with a pharmaceutical carrier.

Also disclosed are methods of inhibiting 21-Ohase gene expression comprising administering TR4 protein or a functional equivalent thereof.

Also disclosed are methods, wherein TR4 protein binding to NBRE AGGTCA is reduced, wherein the NBRE is located at −228 of the 21-Ohase gene, wherein the 21-Ohase gene expression occurs in a human, mouse, or bovine cell.

Disclosed are methods of inhibiting transcription of a gene comprising administering TR4 such that TR4 binds a monomeric binding site associated with the gene.

Disclosed are methods of identifying an inhibitor of 21-Ohase expression comprising forming an incubation mixture comprising a potential inhibitor and a nucleic acid comprising sequence of 21-Ohase gene in double stranded form, and assaying whether the potential inhibitor decreases the amount of TR2, TR4, or combination thereof bound to 21-OHase, and selecting those potential inhibitors that bind 21 Ohase sequence.

Disclosed are methods of producing an inhibitor of 21-Ohase gene expression, comprising identifying the inhibitor of 21-Ohase expression comprising forming an incubation mixture comprising a potential inhibitor and a nucleic acid comprising 21-Ohase nucleic acid in double stranded form, and assaying whether the potential inhibitor decreases the amount of TR2, TR4, or combination thereof bound to 21-Ohase nucleic acid, and selecting those potential inhibitors that bind 21-Ohase nucleic acid.

Disclosed are methods of manufacturing an inhibitor of 21-Ohase gene expression comprising synthesizing the potential inhibitor disclosed herein.

Also disclosed are methods of manufacturing a potential inhibitor 21-Ohase comprising admixing the potential inhibitor(s) disclosed herein with a pharmaceutical carrier.

Also disclosed are methods of inhibiting RA induced cellular activity comprising administering a composition that inhibits TR4 function.

Disclosed are methods, wherein the composition is an antisense nucleic acid, and/or wherein the cellular activity is differentiation, cell cycle progression, cell morphology, or cell apoptosis.

Disclosed are systems for testing the effect of compounds on RA induced cellular activity comprising a cell expressing P19 cells an inhibitor of T4, T2, or a combination thereof

Also disclosed are systems and methods, wherein the inhibitor is controlled by an inducible system, wherein the inducible system is a tetracycline inducible system, wherein the inducible system comprises a first plasmid and a second plasmid, wherein the first plasmid comprises a transcriptional silencer protein under the control of a constitutive promoter, wherein the first plasmid further comprises sequence encoding a poly A tail or a nuclear localization signal, wherein the promoter is a CMV promoter, wherein the transcriptional; silencer protein is TetR-KRAB, wherein the second plasmid comprises a TR4 antisense molecule a TetR-KRAB site, and a strong constitutive promoter, wherein there are 7 TetR-KRAB sites, wherein the first and second plasmid further comprise a selection marker, wherein the composition inhibits the expression of TR4, and/or wherein the composition is an antisense molecule of TR4.

Disclosed are methods of modulating retinoic acid induced apoptosis, comprising administering a modulator of TR4 activity.

Also disclosed are methods, wherein the modulator inhibits the RA induced apoptotic activity, wherein the inhibitor reduces the expression of TR4, wherein the inhibitor is antisense molecule that interacts with TR4 mRNA.

Also disclosed are methods method of reducing RA mediated transactivation in a cell comprising administering TR2 to the cell.

Also disclosed are methods, wherein the cell is a human HaCaT Keratinocyte, wherein the cell is a CV1 cell, and/or wherein the RA mediated transactivation is reduced by at least 44%

Also disclosed are methods of reducing RA mediated transactivation in a cell comprising administering TR4 to the cell, wherein the cell is a human HaCaT Keratinocyte, and/or wherein the RA mediated transactivation is reduced by at least 47%.

Disclosed are methods of regulating transactivation activity of the Wy-14643-mediated peroxisome proliferator-activated receptor a (PPARa) comprising administering TR4.

Also disclosed are methods of regulating the expression of peroxisome proliferator-activated receptor a (PPARa) comprising contacting the PPRE of peroxisome proliferator-activated receptor a (PPARa) with TR2 or TR4, and/or methods wherein the PPRE has the sequence GTCGACAGGGGACCAGGACAaAGGTCACGTTCGGGAGTCGAC or a functional homolog.

Disclosed are methods of regulating the expression of TR2 and TR4 comprising administering Wy-14643.

Disclosed herein TR2 can interact with a number of different response elements.

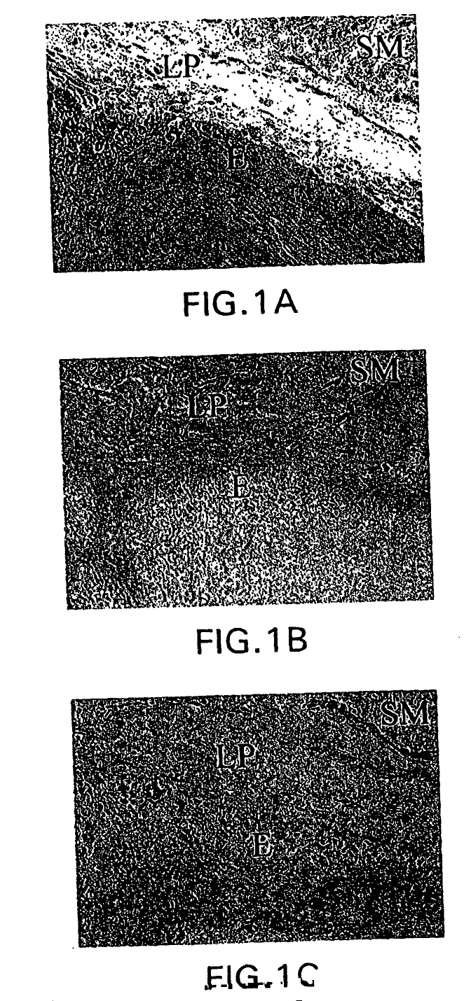

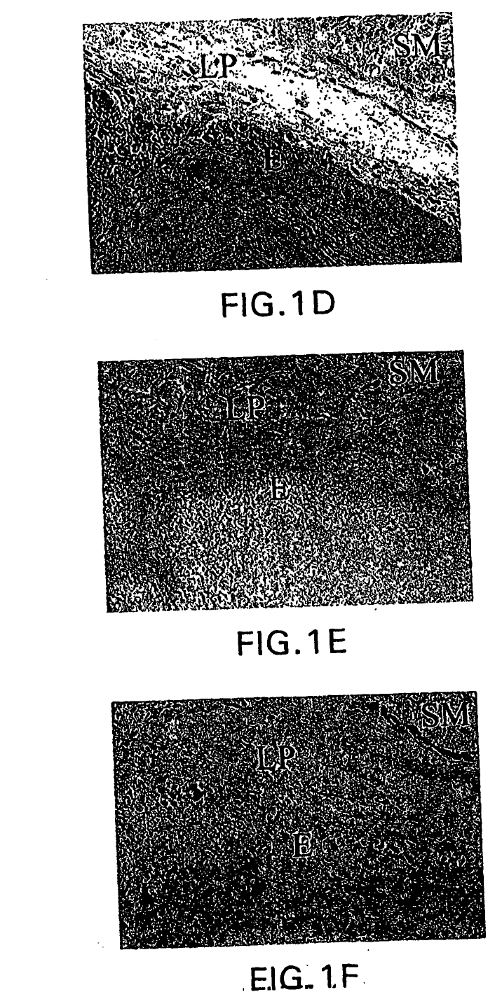

Disclosed herein TR4 can interact with a number of different response elements. TR4 can recognize and bind to tandem repeats of the AGGTCA motif in its target genes (Lin, D. L. et al. (1998) Endocrine 8, 123-134; Lee, H.-J. et al. (1995) J. Biol. Chem. 270, 30129-30133). The binding preference of the TR4 to synthetic DRs (FIG. 1A) is in the order of DR6>DR5>DR1>DR3>DR2>DR4 with the IC₅₀ ranging from 0.08 to 0.24 ng (FIG. 1B).

For natural DRs, however, the TR4 has a different preference in the order of CRBPII (DR1)>SV40+55 (DR2)>TRE (DR4)>RAREβ(DR5)>VDRE (DR3)>ERE (palindrome) with the IC₅₀ varying from 0.023 to 85 ng (FIG. 1C).

For synthetic HREs, there was only a 2 fold difference in the binding affinity among DR1 to DR5. This indicates that the TR4 has a broad and non-discriminating ability to recognize synthetic DRs. The binding affinity for TR4 and natural CRBPII had an 87 fold difference in binding affinity between it and the other HREs. In addition to direct repeats, the TR4 bound to palindromic ERE poorly, suggesting that direct repeats of the AGGTCA consensus motif function more effectively as HREs for the TR4 orphan receptor. Thus the variety in binding preference of the TR4 to different natural HREs strongly implies that not only the space between the direct repeats, but also the motif sequence, contributed to the specific binding of the TR4.

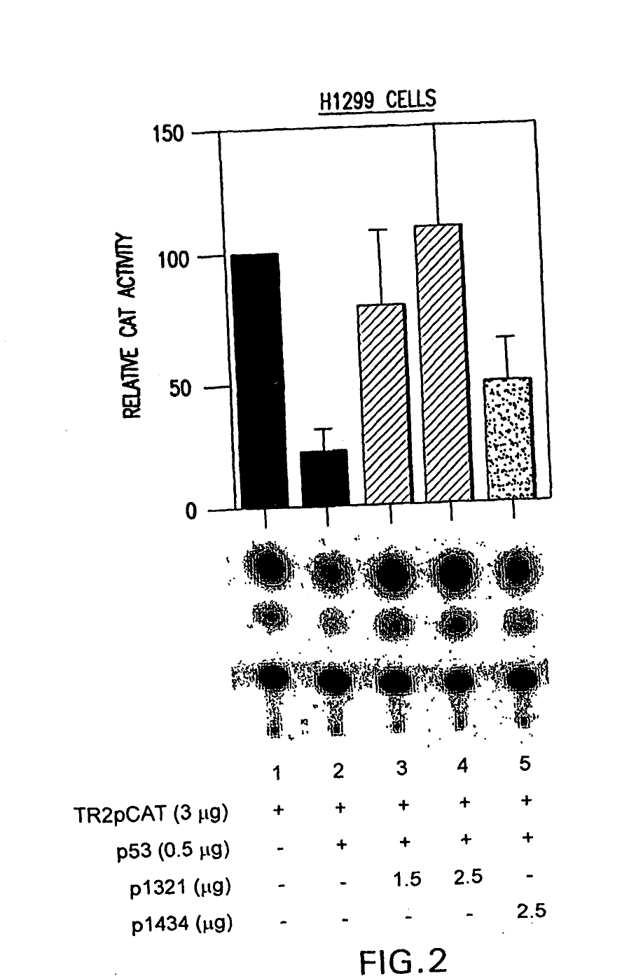

TR4 can bind to a monomer response element. TR4 can bind to a monmeric double stranded NBRE oligonucleotide as a probe. TR4 binds to a monomeric response element as a single binding component, only one TR4 per binding site, and has dissociation constants (K_{d}) of 0.2 nM for the NBRE (FIG. 2B). TR4 can form dimers during DNA binding, while only one of them has specific DNA contacts.

TR4 can significantly suppress the luciferase reporter activity via the −228TR4RE of the 21-OHase gene. TR4 can bind to direct repeats of the AGGTCA core sequence in several response targets (Young, W.-J. et al. (1997) J. Biol. Chem. 272, 3109-3116; Lee, H.-J. et al. (1995) J. Biol. Chem. 270, 30129-30133) with high affinity. Disclosed are TR4 responses in viral gene expression and the cytokine pathway during neurogenesis.

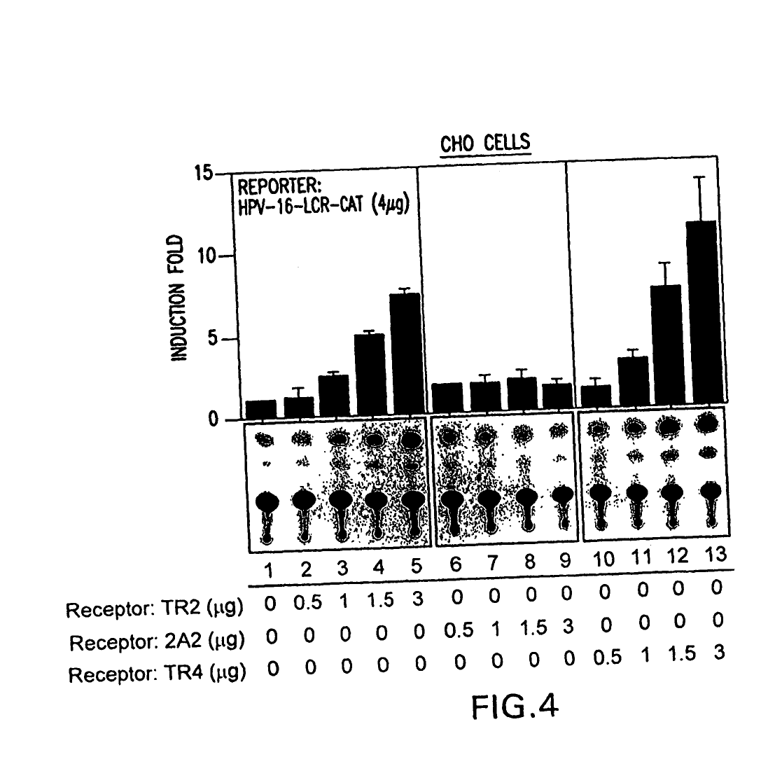

Comparison of the 5′ flanking sequences of the 21-OHase gene among human, mouse, and bovine revealed an important regulatory element which covers the major part of a highly conserved 40-bp region (Parker, K. L. et al. (1986) J. Biol. Chem. 261, 15353-15355). This regulatory region has been demonstrated to be required for expression of 21-OHase in Y-1 adrenal cells. As shown in FIG. 4, this regulatory region in the human gene is located between 253 and 203 nucleotides 5′ of the transcriptional initiation site. Interestingly, the AGGTCA core consensus sequence of a monomeric response element is highly conserved in the center of this regulatory region among all three species. In contrast, the perfect monomeric response element is only present in the human gene, while both mouse and bovine genes vary in the AT-rich preceding region (FIG. 4). This is consistent with TR4 having an additional regulatory effect on human 21-OHase gene expression via this monomeric −228TR4RE. The monomeric TR4 interactions can have a squelching or quenching effect on the transcription.

C. Compositions

Steroid/thyroid hormones function through the action of specific receptor proteins (Mangelsdorf, D. J., et al. (1995) Cell 83, 835-839; Tingley, D. W. (1996) J. NIH Res. 8, 81-88). Steroid/thyroid hormone receptors comprise a huge family of transcriptional factors that regulate complex gene networks in a wide variety of biological processes, such as growth, development, and differentiation (Evans, R. M. (1988) Science 240, 889-895). Members of this superfamily include receptors for steroid hormones, thyroid hormones, vitamin A and D derivatives, as well as a large group of orphan receptors whose cognate ligands remain to be identified. Upon binding to their respective hormonal ligands, the steroid hormone receptors can undergo an activation or transformation step (O'Malley, B. W., and Tsai, M.-J. (1992) Biol. Reprod. 46, 163-167; Truss, M., and Beato, M. (1993) Endocrine Rev. 14, 459-479). The hormone-receptor complex, serving as a trans-regulator, may specifically bind to a cis-acting DNA sequence, known as a hormone response element (HRE), and thereafter regulate the transcription of target genes (Mangelsdorf, D. J., and Evans, R. M. (1995) Cell 83, 841-850). The orphan receptors belong to the nuclear receptor superfamily which mediates extracellular hormonal signals to transcriptional response. The roles of orphan receptors have been linked to development, homeostasis, and diseases. (Mangelsdorf, D. J., and Evans, R. M. (1995) Cell 83(6), 841-50; Enmark, E., and Gustafsson, J. A. (1996) Mol. Endocrinol. 10(11), 1293-307; Giguere, V. (1999) Endocr. Rev. 20(5), 689-725) Orphan receptor is used to group receptor-like proteins that have no known ligand or function (Mangelsdorf, D. J., and Evans, R. M. (1995) Cell 83, 841-850; O'Malley, B. W., and Conneely, O. M. (1992) Mol. Endocrinol. 6, 1359-1361).

1. TR2

The human TR2 orphan receptor (TR2), a member of the nuclear hormone receptor superfamily, was cloned from human testis and prostate cDNA libraries and has no previously identified ligand(s) (Chang, C., et al. (1989) Biochem. Biophys. Res. Commun. 165, 73541; Chang, C. & Kokontis, J. (1988) Biochem. Biophys. Res. Commun. 155, 971-7). In the screening, an oligonucleotide probe homologous to the highly conserved DBD of the glucocorticoid receptor was used (Chang, C., and Kokontis, J. (1988) Biochem. Biophys. Res. Commun. 155, 971-977). TR2 is mapped to locate on chromosome 12q22 (Lin, D. L., et al. (1998) Endocrine 8, 123-34), known to be frequently deleted in various tumors, including testicular and ovarian germ cell tumors (Faulkner, S. W. & Friedlander, M. L. (2000) Gynecol. Oncol. 77, 283-8; Murty, V. V., et al. (1996) Genomics 35, 562-70). Four RNA isoforms, TR2-5, -7, -9, and -11, have been identified. While TR2-11 encodes the full-length receptor, TR2-5, -7, and -9 encode truncated receptors with distinct deletions of ligand-binding domains (LBD) but share identical sequences in the N-terminal and DBD regions (Chang, C., et al. (1989) Biochem. Biophys. Res. Commun. 165, 735-41; Chang, C. et al. (1989) Biochem. Biophys. Res. Commun. 165, 735-741). TR2 has high homology with TR4, which places them in a unique subfamily within the nuclear hormone receptor superfamily (Chang, C., et al. (1994) Proc. Natl. Acad. Sci. USA 91, 6040-4). TR2 is evolutionarily conserved among species from primitive creatures to mammalians, including sea urchin, rainbow trout, axolotl, xenopus, drosophila, mouse, and human (Chang, C., et al. (1989) Biochem. Biophys. Res. Commun. 165, 735-41; Chang, C. & Kokontis, J. (1988) Biochem. Biophys. Res. Commun. 155, 971-7; Kontrogianni-Konstantopoulos, A., et al. (1996) Dev. Biol. 177, 371-82; Zelhof, A. C., et al. (1995) Proc. Natl. Acad. Sci. USA 92, 10477-81; Wirtanen, L., et al. (1997) Differentiation 62, 159-70; Huard, V. & Seguin, C. (1998) DNA Seq 9, 113-20; Le Jossic, C. & Michel, D. (1998) Biochem Biophys Res Commun 245, 64-9). The facts that TR2 is broadly expressed in many tissues throughout development starting at as early as midgestation stage (Lee, C. H., et al. (1996) Mol. Reprod. Dev. 44, 305-14; Young, W. J., et al. (1998) J. Biol. Chem. 273, 20877-85; Lee, H. J., et al. (1996) J. Biol. Chem. 271, 10405-12; Lin, T. M., et al. (1995) J. Biol. Chem. 270, 30121-8) and that drosophila with null mutations of DHR78 nuclear receptor, a homolog of human TR2, is lethal at the third-instar larval stage with severe defects in ecdysteroid-triggered metamorphosis (Fisk, G. J. & Thummel, C. S. (1998) Cell 93, 543-55) are consistent with the biological importance of TR2 being involved in the development process. It has been emphasized that with prominent expression throughout the active proliferating zones of the neural areas and the sensory nerve-targeted organs and the testes during development, TR2 may exert an important role in the early development of the nervous system and the male reproductive system (Lee, C. H., et al. (1996) Mol. Reprod. Dev. 44, 305-14; Young, W. J., et al. (1998) J. Biol. Chem. 273, 20877-85; Lee, H. J., et al. (1996) J. Biol. Chem. 271, 10405-12; Lin, T. M., et al. (1995) J. Biol. Chem. 270, 30121-8). Also, it has been shown that TR2 is primarily expressed in the mouse testis, particularly in the developing germ cells, indicating a role of TR2 in spermatogenesis (Lee, C. H., et al. (1996) Mol. Reprod. Dev. 44, 305-14; Lee, C. H., et al. (1995) Genomics 30, 46-52).

Using the known TR2 HRE sequence as a guide, it was determined that Th2 has modulatory effects on several signaling pathways, such as those involving retinoic acid (Lin, T. M. et al. (1995) J. Biol. Chem. 270, 30121-30128), thyroid hormone (Chang, C., and Pan, H. (1998) Mol. Cel. Biochem. 189, 195-200), and ciliary neurotrophic factor (Young, W. et al. (1998) J. Biol. Chem. 273, 20877-20885). Moreover, expression of the erythropoietin gene (Lee, H. J., and Chang, C. (1995) J. Biol. Chem. 270, 5434-5440), aldolase gene (Chang, C., and Lee, H. (1997) Biochem. Biophys. Res. Commun. 235, 205-211), histamine H1 receptor (Lee, H., and Chang, C. (1999) Mol. Cel. Biochem. 194, 199-207), and Simian Virus 40 (SV40) (Lee, H. J. et al. (1996) J. Biol. Chem. 271, 10405-10412) is also regulated by TR2. Disclosed herein, a TR2 DR4 consensus site within the long control region (LCR) of the human papilloma virus type 16 (HPV-16) has been found. Also disclosed herein it is shown that TR2 is able to bind the consensus response element (DR4RE) within the HPV-16 gene, and modulate HPV-16 expression.

In cell line models, information regarding TR2 function, in terms of transcription activity, has been demonstrated by many studies. TR2 functions as a transcription factor that binds to its consensus response element (AGGTCA) in a direct repeat (DR) orientation (AGGTCA(n)ₓAGGTCA, x=1-6) (Lin, T. M., et al. (1995) J. Biol. Chem. 270, 30121-8). New TR2 target genes are continually being discovered, such as cellular retinol-binding protein II (CRBPII), retinoic acid receptor β (RARβ), SV40, erythropoietin, histamine H1 receptor, muscle-specific aldolase A, and ciliary neurotrophic factor receptor (CNTFR) (Young, W. J., et al. (1998) J. Biol. Chem. 273, 20877-85; Lee, H. J., et al. (1996) J. Biol. Chem. 271, 10405-12;

Lin, T. M., et al. (1995) J. Biol. Chem. 270, 30121-8, Lee, H. J. & Chang, C. (1995) J. Biol. Chem. 270, 5434-40; Lee, H. J., et al. (1995) J. Biol. Chem. 270, 30129-33; Chang, C. & Pan, H. J. (1998) Mol. Cell Biochem. 189, 195-200; Lee, H. J., et al. (1999) Mol Cell Biochem 194, 199-207), suggesting that TR2 has abroad range of biological functions. In terms of the regulation of TR2 expression, TR2 can be induced during neuronal differentiation in P19 embryonic carcinoma cells stimulated by ciliary neurotrophic factor (CNTF). In return, TR2 activates its target gene, CNTFR, expression which mediates CNTF signaling and is required for the motor neuron development (Young, W. J., et al. (1998) J. Biol. Chem. 273, 20877-85; DeChiara, T. M., et al. (1995) Cell 83, 313-22). These may provide a linkage between TR2 and neurogenesis. The tumor suppressor genes, p53 and Rb, that induce cell cycle arrest can down-regulate TR2 expression in cells after ionizing radiation and in cells overexpressing p53 or Rb (Mu, X., et al. (2000) J. Biol. Chem. 275, 23877-83; Lin, D. L. & Chang, C. (1996) J. Biol. Chem. 271, 14649-52). TR2 can then go through a feed-back control mechanism to induce HPV-16 E6 and E7 target gene expression that are known to enhance the P53 protein degradation and inactivate the Rb function, respectively (Mu, X., et al. (2000) J. Biol. Chem. 275, 23877-83, Collins, L. L., et al. (2001) J Biol Chem 276, 27316-21). TR2 is, therefore, thought to be involved in cell cycle regulation.

In addition to functioning as a transcription regulator, TR2 can modulate other signaling via different mechanisms. For example, TR2 suppresses RXR- and RXR/RAR-mediated transcription by binding to the same DNA response element (DRE) with a higher binding affinity (Lin, T. M., et al. (1995) J. Biol. Chem. 270, 30121-8) and represses thyroid receptor α/RXR signaling by competing for limited amounts of DREs (Chang, C. & Pan, H. J. (1998) Mol. Cell Biochem. 189, 195-200). TR2 can also exert its suppressive effects via the recruitment of class I and class II histone deacetylases (HDAC) (Franco, P. J., et al. (2001) Mol Endocrinol 15, 1318-28).

Nucleic Acids that encode various TR2s. There are many variants and allelic and homolog molecules of TR2. Genbank accession numbers for an exemplary set are provided here. Each of these sequences is herein incorporated by reference, at least for material related to the sequence. It is also understood that one of skill in the art would recognize the various TR2 proteins encoded by the nucleic acids, where protein sequence is not provided. A representative list of TR2 genes and related sequences can be found at Genbank Accession Nose: NT_{—}031693, NM_{—}003807, BM313468, BM272414, BM272208, NM_{—}003297, BF476378, BF223014, BF109885, BE856797, 12: AW743650, AU076765, AW299455, AW272476, AW105139, AW073142, AW002180, AI983624, AF171055, AM893903, AI864325, AI686942, AI653325, AI507032, AI431858, AI370806, AI341113, AI203072, AI385609, AI379335, AI3708071, AA884437, AA770397, AI242989, AI127957, AI153653, AI089445, AI081737, AI089220, U30482, AI050052, AI005665, AA227068, AA226914, AA656392, AA641155, AA593861, AA558488, AA454474, AA411285, AA381676, AA375076, AA300579, W36063, W39474, W38377, U19026, H75390, H68838, H68990, T27625, R54467, R52304, T95992, T95892, T84513, M29959, M29960, and M21985. Unless specifically indicated, TR2 refers to any TR2 molecule. It is understood that TR2 can be divided into, for example, a DNA binding domain (DBD, a ligand binding domain (LBD), and an activation domain (AD), just like opther steroid receptors. These regions are readily identifiable and are disclosed herein. It is also understood that a TR2 lacking a functional activation domain can act as an inhibitor of normal TR2 function, by for example, competing with DNA binding. Thus, for example, a molecule comprising just the DNA binding domain, and lacking a functional activation domain can be used as an inhibitor of TR2 or TR 4 function. Likewise with molecules comprising the DNA binding domain, but lack a functional ligand binding domain. Any molecule that binds the TR2/TR4 element and prevents transactivation by TR2 or TR4 can be considered an inhibitor of TR2 or TR4 activity. These molecules can be identified using transcription activation assays, as described herein. It is also understood that the various isoforms of TR2, lacking the LBD, 6, 7, and 9 can function as TR2 inhibitors.

2. TR4

The human testicular receptor 4 (TR4) was originally isolated from testes, prostate, and brain cDNA libraries by degenerative polymerase chain reaction cloning (Chang, C. et al. (1994) Proc. Natl. Acad. Sci. USA. 91(13), 6040-4). While TR4 shares the structural features of nuclear receptors, no ligand has yet been previously identified and it is therefore considered an orphan receptor.

TR4 directly regulates transcription through binding to a direct repeat (DR) of a AGGTCA core element separated by a variable number of nucleotides. (TR2 and TR4 can bind to AGGTCA direct repeats (DRx; AGGTCA (n)ₓ AGGTCA, x=0-6) (Lee, Y.-F. et al. (1997) J. Biol. Chem. 272, 12215-12220; Chang, C., and Pan, H.-J. (1998) Molecular and Cellular Biochemistry; Young, W.-J. et al. (1997) J. Biol. Chem. 272, 3109-3116; Young, W.-J. et al. (1998) J. Biol. Chem. 273, 20877-20885; Lin, T.-M. et al. (1995) J. Biol. Chem. 270, 30121-30128; Lee, Y.-F. et al. (1998) J. Biol. Chem. 273, 13437-13443; Lee, H.-J., and Chang, C. (1995) J. Biol. Chem. 270, 5434-5440; Lee, H.-J. et al. (1995) J. Biol. Chem. 270, 30129-30133; Lee, H.-J. et al. (1996) J. Biol. Chem. 271, 10405-10412)). TR4 functions as a transcriptional activator when bound to the DR separated by four nucleotides (a DR-4 element) (Chang, C. et al. (1994) Proc. Natl. Acad. Sci. USA. 91(13), 6040-4). However, TR4 functions as a transcriptional repressor when bound to DR-1, DR-2, DR-3, or DR-5 type (Lee, Y. F. et al. (1998) J. Biol. Chem. 273(22), 13437-43; Lee, H. J. et al. (1995) J. Biol. Chem. 270(50), 30129-33; Lee, Y. F. et al. (1999) J. Biol. Chem. 274(23), 16198-205). The differential spacings between the core elements cause TR4 to adopt different conformations and alter the ability of TR4 to interact with coregulators (Lee, Y. F. et al. (1999) J. Biol. Chem. 274(23), 16198-205). Consistent with its neuronal localization, TR4 also induces the transcription of the cytokine receptor, which is a ciliary neurotrophic factor receptor (Young, W. J. et al. (1997) J. Biol. Chem. 272(5), 3109-16).

In addition to direct transcriptional regulation, TR4 can also modulate other nuclear receptors' transactivation. Previous studies have indicated that TR4 can compete for binding to the hormone response elements of retinoic acid receptor (RAR), retinoid X receptor (RXR) (Lee, Y. F. et al. (1998) J. Biol. Chem. 273(22), 13437-43) and vitamin D receptor (VDR) (Lee, Y. F. et al. (1999) J. Biol. Chem. 274(23), 16198-205) to suppress RAR/RXR- or VDR-mediated transcription. TR4 may also inhibit peroxisome proliferator activated receptor alpha (PPARα) induced transactivation by competitive binding to PPAR response elements and through competition for coactivators such as RIP140 (Yan, Z. H. et al. (1998) J. Biol. Chem. 273(18), 10948-57). The AR-TR4 interaction could then result in the mutual suppression of AR- or TR4-mediated transcription (Lee, Y. F. et al. (1999) Proc. Natl. Acad. Sci. USA. 96(26), 14724-9). Previous reports have linked TR4 function to neurogenesis (Young, W. J. et al. (1997) J. Biol. Chem. 272(5), 3109-16) and spermatogenesis (Lee, C. H. et al. (1998) J. Biol. Chem. 273(39), 25209-15). TR4 has been demonstrated to suppress many other receptors' transactivation, such as VDR, RAR, RXR, and PPAR (Lee, Y. F. et al. (1998) J. Biol. Chem. 273(22), 13437-43, Lee, Y. F. et al. (1999) J. Biol. Chem. 274(23), 16198-205; Yan, Z. H. et al. (1998) J. Biol. Chem. 273(18), 10948-57). The suppression mechanism for these receptors' transactivation has been demonstrated through the competition of TR4 with those receptors' ability to bind their hormone response elements.

The human TR4 cDNA shares structural homology with members of the steroid hormone receptor superfamily (Chang, C. et al. (1994) Proc. Natl. Acad. Sci. USA. 91, 6040-6044). The TR4, also named as TAK1 (Hirose, T. et al. (1994) Mol. Endocrinol. 8, 1667-1680), is most closely related to the previously identified TR2 orphan receptor (Chang, C., and Kokontis, J. (1988) Biochem. Biophys. Res. Commun. 155, 971-977; Chang, C. et al. (1989) Biochem. Biophys. Res. Commun. 165, 735-741; Gronemeyer, H., and Laudet, V. (1995) Protein Profile 2, 1173-1308), forming subfamily within the superfamily of steroid receptors. Recently, the TR4 was designated as the TR2β, while the TR2 orphan receptor was referred to as TR2α (Mangelsdorf, D. J., and Evans, R. M. (1995) Cell 83, 841-850). The mouse TR4 cDNA has been cloned from mouse testis by reverse transcription-PCR (Young, W.-J. et al. (1997) J. Biol. Chem. 272, 3109-3116). Subsequently, the human TR4 gene has been mapped to chromosome 3p24.3 (Lin, D. L. et al. (1998) Endocrine 8, 123-134).

TR4 encodes a 67 kDa protein (Chang, C. et al. (1994) Proc. Natl. Acad. Sci. USA 91, 6040-6044). The P-box sequence of the DNA binding domain (DBD), TR4 is classified as a member of the estrogen receptor and thyroid hormone receptor subfamily, which can recognize the hormone response elements (HREs) composed of the AGGTCA motif. Examples of HREs with this motif include those of the retinoic acid receptor (RARE), retinoid X receptor (RXRE) (4), thyroid hormone receptor (T₃RE) (Lee, Y.-F. et al. (1997) J. Biol. Chem. 272, 12215-12220) and vitamin D receptor (VDRE)¹. In this case, TR4 may interfere with other steroid hormone pathways by binding to the same HREs. Several in vitro studies have demonstrated that TR4 acts as a regulator of various steroid/thyroid hormone pathways (Lee, Y.-F. et al. (1997) J. Biol. Chem. 272, 12215-12220; Young, W.-J. et al. (1997) J. Biol. Chem. 272, 3109-3116; Lee, H.-J. et al. (1995) J. Biol. Chem. 270, 30129-30133; Young, W.-J. et al. (1998) J. Biol. Chem. 273, 20877-20885).

In situ hybridization analysis shows that TR4 is highly expressed in adult mouse brain especially in the regions in which cells undergo active proliferation and in the granule cells of the hippocampus and cerebellum (Chang, C. et al. (1994) Proc. Natl. Acad. Sci. USA. 91(13), 6040-4). It has been demonstrated that TR4 inhibits the retinoic acid (RA) pathway that is highly involved in the development of the nervous system (Young, W.-J. et al. (1998) J. Biol. Chem. 273, 20877-20885). In contrast, TR4 enhanced the transactivation activity of the ciliary neurotrophic factor receptor (CNTFR) gene, whose expression pattern is restricted to nervous tissues and is highly similar to that of TR4, via binding to CNTFR-DR1 (Young, W.-J. et al. (1997) J. Biol. Chem. 272, 3109-3116). It was found that treatment of cells with RA would increase TR4 amounts at both RNA and protein levels (Lee, Y.-F. et al. (1998) J. Biol. Chem. 273, 13437-13443). The TR4 increase was also observed in CNTF-treated mouse P19 teratocarcinoma cells (Young, W.-J. et al. (1998) J. Biol. Chem. 273, 20877-20885). The data from both in situ and in vitro studies suggest that TR4 may be involved in the regulation of differentiation of neuron cells.

In situ hybridization analysis has demonstrated that TR4 is expressed in a complex spatiotemporal pattern. In the development of neurons, TR4 transcripts were detected throughout the neural tube at early stages of embryo development, and were subsequently restricted to the regions where cells were rapidly proliferating in the later stages of the embryo (Young, W.-I. et al. (1997) J. Biol. Chem. 272, 3109-3116). Consistent with in situ analysis of mouse embryos, the TR4 transcripts were expressed higher in the S-phase than in G1 and G2/M phases, determined by testing of elutriated P19 cell fractions.

In addition, the expression of the TR4 transcripts occurs widely in many mouse tissues, including the central nervous system and peripheral organs such as the adrenal gland, spleen, thyroid gland, and prostate (Yoshikawa, T. et al. (1996) Endocrinol. 137, 1562-1571; Young, W.-J. et al. (1997) J. Biol. Chem. 272, 3109-3116). These data are consistent with TR4 playing a role in neurogenesis and neuronal maturation.

There are many variants and allelic and homolog molecules of TR4. Genbank accession numbers for an exemplary set are provided here. Each of these sequences is herein incorporated by reference, at least for material related to the sequence. It is also understood that one of skill in the art would recognize the various TR4 proteins encoded by the nucleic acids, where protein sequence is not provided. A representative list of TR4 genes and related sequences can be found at Genbank Accession Nos: NM_{—}017323 Rattus norvegicus TR4 orphan receptor (Tr4), mRNA; AV327704 RIKEN full-length enriched, adult male medulla oblongata Mus musculus cDNA clone 6330436D07 3′ similar to L27513 Rat TR4 orphan receptor mRNA, mRNA sequence; BF439121 Soares_NSF_F8_{—}9W_OT_PA_P_S1 Homo sapiens cDNA clone IMAGE:3270267 3′ similar to SW:TR4_HUMAN P49116 ORPHAN NUCLEAR RECEPTOR TR4;, mRNA sequence; NM_{—}003298, Homo sapiens nuclear receptor subfamily 2, group C, member 2 (NR2C2), mRNA; U59454 Rattus norvegicus orphan receptor TR4-NS (TR4) gene, unspliced intron, partial sequence; and partial cds; AW169955 Soares_NFL_T_GBC_S1 Homo sapiens cDNA clone IMAGE:2659198 3′ similar to SW:TR4_HUMAN P49116 ORPHAN NUCLEAR RECEPTOR TR4;, mRNA sequence; AI924957 wn26c02.x1 NCI_CGAP_Gas4 Homo sapiens cDNA clone IMAGE:2446562 3′ similar to SW:TR4_HUMAN P49116 ORPHAN RECEPTOR TR4;, mRNA sequence; AI571166 tn85h11.x1 NCI_CGAP_Ut2 Homo sapiens cDNA clone IMAGE:2176389 3′ similar to SW:TR4_HUMAN P49116 ORPHAN RECEPTOR TR4;, mRNA sequence; AA781743 ai60f03.s1 Soares_testis_NHT Homo sapiens cDNA clone 1375229 3′ similar to SW:TR4_HUMAN P49116 ORPHAN RECEPTOR TR4;, mRNA sequence; AI221141qg91c11.x1 Soares_NFL_T_GBC_S1 Homo sapiens cDNA clone IMAGE:1842548 3′ similar to SW:TR4_HUMAN P49116 ORPHAN RECEPTOR TR4;, mRNA sequence; AI218732, oo07b04.x1 Soares_NSF_F8_{—}9W_OT_PA_P_S1 Homo sapiens cDNA clone IMAGE: 1565455 3′ similar to SW:TR4_HUMAN P49116 ORPHAN RECEPTOR TR4;, mPNA sequence; U59456 Rattus norvegicus nuclear hormone receptor rTR4alpha1 (TR4) mRNA, exon Q, partial cds; AH006640 Human orphan receptor TR4 (TR4) gene, intron N1, 5′ sequence; U40267 Human orphan receptor TR4 (TR4) gene, intron N3, 3′ sequence; U40266 Human orphan receptor TR4 (TR4) gene, intron N3, 5′ sequence; U40150 Human orphan receptor TR4 (TR4) gene, intron N2, 3′ sequence; U40149 Human orphan receptor TR4 (TR4) gene, intron N2, 5′ sequence; U40148 Human orphan receptor TR4 (TR4) gene, intron N1, 3′ sequence; U39639 Human orphan receptor TR4 (TR4) gene, intron N1, 5′ sequence; AI024815 ov35f10.x1 Soares_testis_NHT Homo sapiens cDNA clone IMAGE:1639339 3′ similar to SW:TR4_HUMAN P49116 ORPHAN RECEPTOR TR4;, mRNA sequence AI018200, Soares_NFL_T_GBC_{—S}1 Homo sapiens cDNA clone IMAGE:1626410 3′ similar to SW:TR4_HUMAN P49116 ORPHAN RECEPTOR TR4;, mRNA sequence; U30482 Mus musculus TR2 (mTR2) mRNA, complete cds; U32939 Mus musculus TR4 mRNA, partial cds; U59455 Homo sapiens TR4 gene, unspliced intron, 3′sequence; L27513 Rat TR4 orphan receptor mRNA, complete cds; and L27586 Human TR4 orphan receptor mRNA, complete cds. Protein sequences can be found at for example, Genbank Accession Nos: NP_{—}003289, AAB91433, P55094, AAC52777, AAC50677, AAC50676, AAC50675, AAC50674, AAC50673, AAC29502, AAC18408, AAA21475 and AAA21474 which are herein incorporated by reference at least for the disclosed sequences. Unless specifically indicated, TR4 refers to any TR4 molecule.

It is understood that TR4 can be divided into, for example, a DNA binding domain (DBD, a ligand binding domain (LBD), and an activation domain (AD), just like opther steroid receptors. These regions are readily identifiable and are disclosed herein. It is also understood that a TR4 lacking a functional activation domain can act as an inhibitor of normal TR4 function, by for example, competing with DNA binding. Thus, for example, a molecule comprising just the DNA binding domain, and lacking a functional activation domain can be used as an inhibitor of TR2 or TR 4 function. Likewise with molecules comprising the DNA binding domain, but lack a functional ligand binding domain. Any molecule that binds the TR2/TR4 element and prevents transactivation by TR2 or TR4 can be considered an inhibitor of TR2 or TR4 activity. Any molecule that binds the TR2/TR4 element and prevents transactivation by TR2 or TR4 can be considered an inhibitor of TR2 or TR4 activity. These molecules can be identified using transcription activation assays, as described herein.

3. HPV

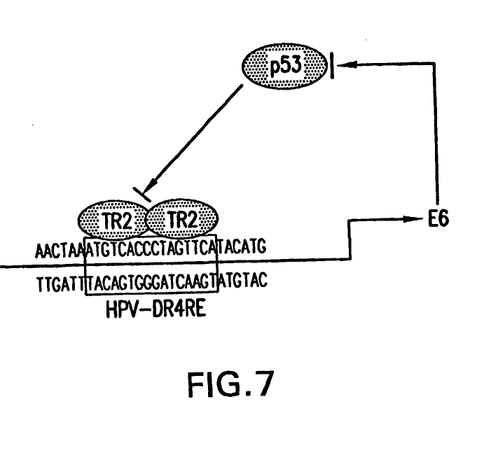

The HPV-16 genome consists of an approximately 850 bp regulatory long control region (LCR), as well as early (E6, E7, E1, E2, E4 and E5) and late (L2 and L1) open reading frames (ORF) (Seedorf, K. et al. (1985) Virology 145, 181-185). Upstream of the HPV-16 E6-E7 gene region, there exist E2-responsive core sequences, as well as a short enhancer element which responds to E2-independent cellular factors in keratinocytes (Cripe, T. P. et al. (1987) EMBO J. 6, 3745-3753). The E2 ORF encodes two trans-acting factors that have the potential to bind upstream, and modulate expression of the E6 and E7 genes. The long E2 gene product binds E2-responsive core sequences upstream of the E6-E7 promoter and has a transactivating effect. In contrast, there is a C-terminal E2 gene product which inhibits E2-independent transactivation, as well as the keratinocytic cellular factor dependent response (Cripe, T. P. et al. (1987) EMBO J. 6, 3745-3753). Viral integration in the infected host often occurs in the region of the E1-E2 ORFs, disrupting transcriptional regulation by E2. (Munger, K., et al. (1989) J. Virology 63, 4417-4421). Disruption of both the activation and suppression functions of the E2 gene products allows upregulation of upstream enhancer-mediated, cellular factor-dependent transactivation of the E6 and E7 oncogenes (Cripe, T. P. et al. (1987) EMBO J. 6, 3745-3753). The HPV-16 protein E6 is capable of binding to the p53 tumor suppressor protein (Werness, B. A. et al. (1990) Science 248, 76-79), and has been shown to stimulate ubiquitin-dependent degradation of p53 (Scheffner, M. et al. (1990) Cell 63, 1129-1136). Further, on a functional level, HPV-16 E6 expression has been shown to disrupt the p53-mediated pathway, which is induced in response to DNA damage. This suggests that the E6 oncogene may, through its association with p53, disrupt a tumor suppressive response and therefore contribute to the accumulation of genetic changes often associated with tumorigenesis (Kessis, T. D. et al. (1993) Proc. Natl. Acad. Sci. U.S.A. 90, 3988-3992). With TR2, a transcription factor that induces HPV-16 gene expression, and that is repressed by p53, the potential for cell cycle disruption and for propagation of the viral infection increases. By inducing expression of the HPV-16 early gene E6, TR2 lessens the regulatory effect exerted by p53 on both TR2 itself, as well as on the cell cycle in general. Without the tumor suppressive regulation afforded by p53, the human papilloma virus is able to infect growth-arrested cells and more readily induce proliferation of differentiated cells (Syrjanen, S. M., and Syrjanen, K. S. (1999) Ann. Med. 31, 175-187). Disclosed herein TR2 is involved in a positive feedback loop with HPV-16, as depicted in FIG. 7. TR2 induces HPV-16 E6, and E6, in turn, reduces the available p53. In this way, p53-mediated cell cycle regulation, as well as TR2 repression, is attenuated. As a result, more TR2 will be available to continue to upregulate E6 expression. As the cell cycle continues, more and more cells will undergo HPV infection, and loss of p53-mediated regulation of proliferation will occur. This process increases the potential for accumulation of mutations that may eventually lead to malignancy. Our studies reported here indicate that TR2 is likely to be expressed in the cell types prone to HPV-16 infection (the squamous epithelial cells of the vagina and cervix), and that a TR2 response element has been identified in the HPV-16 LCR. Also disclosed herein is the binding of TR2 to the HPV-16-DR4RE and the subsequent upregulation of HPV-16 gene transcription.

The human papilloma virus is a common sexually transmitted pathogen that is linked to increased risk for the development of cervical neoplasia, one of the most common cancers in women throughout the world. HPV is thought to contribute to the development of 10-15% of all human cancers (Syrjanen, S. M., and Syrjanen, K. J. (1999) Ann. Med. 31, 175-187). Papillomaviruses are epitheliotropic organisms, and therefore result in chronic infection of the skin and mucous membranes. The most common site of infection of genital HPVs in women is the stratified squamous epithelium at the squamocolumnar junction of the cervix. Over 70 HPV types have been identified, and each is classified as either low risk (eg. HPV-6, HPV-11) based on association with benign lesions, or high risk (eg. HPV-16, HPV-18) based on association with cervical carcinoma. Over 90% of cervical cancers show high risk HPV infection, and over 20% of genital HPV types are associated with cervical cancer; high risk HPV-16 is the predominant tumor-associated type (Bosch, F. X. et al. (1995) J. Natl. Cancer Inst. 87, 796-802).

A common element in pathways involving either TR2 or HPV is the p53 tumor suppressor. The p53 protein is a transcription factor that binds to a specific regulatory sequence and modulates the expression of various target genes such as p21 waf1/cip1, bax, mdm2 and cyclin G (reviewed in Syrjanen, S. M., and Syrjanen, K. J. (1999) Ann. Med. 31, 175-187). Through regulation of its target genes, p53 is able to suppress cell cycle progression in response to DNA damage. As a tumor suppressor, p53 is able to block DNA replication after damage has occurred, allowing time for damage repair or the induction of the onset of apoptosis (Martinez, J. et al. (1991) Genes and Dev. 5, 151-159; Yonish-Rouach, E. et al. (1991) Nature 352, 345-347). This process prevents mutations, induced via DNA-damaging agents, from being replicated and passed on, thus reducing the potential for cellular transformation. p53 expression has been shown to increase after insults such as gamma-irradiation or actinomycin D treatments, which damage DNA directly. Along with p53 induction, cells demonstrate corresponding cell cycle arrest in phase G1 (Kastan, M. B. et al. (1991) Cancer Res. 51, 6304-6311). Relating this to TR2, it has been shown that ionizing radiation represses the expression of the orphan receptor at both transcriptional and translational levels, and that this repression is mediated by p53 (Lin, D., and Chang, C. (1996) J. Biol. Chem. 271, 14649-14652). The TR2 protein expression kinetics in cells after irradiation was opposite that of p53 expression; TR2 levels decreased dramatically 1-2 h after treatment and were restored to pre-treatment levels over time, whereas p53 levels increased soon after treatment and decreased over time. The suggestion that p53 plays a role in the repression of TR2, which is induced by ionizing radiation, was further substantiated by the reduction in TR2 expression after p53 levels were increased in the absence of irradiation, as well as by the lack of TR2 repression when a mutant form of p53 was overexpressed (Lin, D., and Chang, C. (1996) J. Biol. Chem. 271, 14649-14652). It is known that steroid hormone response elements exist within the HPV-16 viral regulatory region. Both progesterone and glucocorticoid have been shown to induce HPV-16 gene expression through receptor association with three glucocorticoid response elements (GREs) within the HPV-16 LCR (Khare, S. et al. (1997) Exptl. Cell Res. 232, 353-360; Gloss, B. et al. (1989) J. Virology 63, 1142-1152; Chan, W. et al. (1989) J. Virology 63, 3261-3269; Mittal, R. et al. (1993) Obstet. Gynecol. 81, 5-12). Disclosed herein TR2 is also able to modulate the expression of HPV-16 genes, and that it does so through binding to a TR2 response element located in the HPV-16 LCR. Also disclosed herein E6 is able to reduce the repression of TR2 by p53, which is likely to occur through the binding of p53 by E6, an event that initiates p53 degradation. Cumulatively, the disclosed data demonstrate a positive-feedback regulatory pathway between TR2 and HPV-16, providing evidence for a novel relationship between a steroid receptor and the HPV-16 DNA tumor virus.

There are many strains of HPV. Genbank accession numbers for an exemplary set are provided here. Each of these sequences is herein incorporated by reference, at least for material related to the sequence. It is also understood that one of skill in the art would recognize the various HPV proteins encoded by the nucleic acids, where protein sequence is not provided.

A representative list of HPV genomes and related sequences can be found at Genbank Accession Nos: NC_{—}001353 Human papillomavirus type 57, complete genome; NC_{—}002627 Human papillomavirus type 87, complete genome; AJ400628 Human papillomavirus type 87 (candidate) complete genome; NC_{—}001526 Human papillomavirus type 16, complete genome; NC_{—}001691 Human papillomavirus type 50, complete genome; NC_{—}001695 Human papillomavirus type 66, complete genome; NC_{—}001694 Human papillomavirus type 61; complete genome; NC_{—}001693 Human papillomavirus type 60, complete genome; NC_{—}001692 Human papillomavirus type 55, complete genome; NC_{—}001690 Protein Human papillomavirus type 48, complete genome; NC_{—}001689 Human papillomavirus type 44, complete genome; NC_{—}001688 Human papillomavirus type 38, complete genome; NC_{—}001687 Human papillomavirus type 37, complete genome; NC_{—}001686 Human papillomavirus type 36, complete genome; NC_{—}001685 Human papillomavirus type 29, complete genome; NC_{—}001684 Human papillomavirus type 28, complete genome; NC_{—}001680 Human papillomavirus type 21, complete genome; NC_{—}001679 Human papillomavirus type 20, complete genome; NC_{—}001459 Human papillomavirus type 65, complete genome; NC_{—}001458 Human papillomavirus type 63, complete genome; NC_{—}001457 Human papillomavirus type 4, complete genome; NC_{—}001444 Human papillomavirus type 5b, complete genome; AF293961 Human papillomavirus type 82 subtype IS39/AE2, complete genome; NC_{—}001352 Human papillomavirus type 2a, complete genome; NC_{—}002676 Human papillomavirus type 84, complete genome; AF293960 Human papillomavirus type 84, complete genome; AF131950 Human papillomavirus candHPV85, complete genome: NC_{—}001676 Human papillomavirus type 54, complete genome; NC_{—}001530 Human papillomavirus type 47, complete genome; AF125673 Human papillomavirus type 16 isolate 16W12E, complete genome; U37488 Human papillomavirus type 54, complete genome; U89349 Human papillomavirus type 18 variant, partial sequence; M32305 Human papillomavirus type 47 (HPV-47) +-sense strand; U40822 Human papillomavirus type 74 E6 protein (E6), E7 protein (E7), and L1 protein (L1) genes, complete cds; U22461 Human papillomavirus type 70 E6 protein (E6), E7 protein (E7), and L1 protein (L1) genes, complete cds; 53: D90252 Human papillomavirus type 5b complete genome; 54: Y15175 Human papillomavirus type 77 E6, E7, E1, E2, E4, L2, and L1 genes; Y15174 Human papillomavirus type 76 E6, E7, E1, E2, E4, L2, and L1 genes; Y15173 Human papillomavirus type 75 E6, E7, E1, E2, E4, L2, and L1 genes; Y15176 Human papillomavirus type 80 E6, E7, E1, E2, E4, L2, and L1 genes; D00204 Human papillomavirus type 19 gene for L1 protein, E6 protein, partial cds; D00205 Human papillomavirus type 25 gene for L1 protein, E6 protein, partial cds; AF014803 Human papillomavirus type 16 mutant early transforming protein E6 (E6) gene, partial cds; D90264 Human papillomavirus (HPV) type 25 E6 gene; D90263 Human papillomavirus (HPV) type 21 E6 gene; D90261 Human papillomavirus (HPV) type 20 E6 gene; D90262 Human papillomavirus (HPV) type 14 E6 gene; S80204 {L1-E6 region, long control region} [Human papillomavirus type 11, HPV-11, Genomic, 627 nt]; S80202. {L1-E6 region, long control region} [Human papillomavirus type 6a, HPV-6a, Genomic Mutant, 707 nt]; S80200 {L1-E6 region, long control region} [Human papillomavirus type 6b, HPV-6b, Genomic, 675 nt]; AH004565 L1, E6 {long control region} [human papillomavirus type 2c HPV-2c, Genomic, 400 nt 2 segments]; S70160 Related Sequences, PubMed, Taxonomy L1, E6 {long control region} [human papillomavirus type 2c HPV-2c, Genomic, 200 nt, segment 2 of 2]; 547085 L1, E6 {long control region} [human papillomavirus type 2c HPV-2c, Genomic, 400 nt 2 segments]; S70159 L1, E6 {long control region} [human papillomavirus type 2c HPV-2c, Genomic, 200 nt, segment 1 of 2]; S60413 Long control region: L1 orf, E6 orf {LCR microheterogeneity} [human papillomavirus type 16 HPV 16, isolate C3, Genomic, 883 nt]; S51110 orf E6 [human papillomavirus HPV, type 16, head and neck tumor, Genomic, 421 nt]; X94164 Human papillomavirus type 72 E6, E7, E1A, E1B, E2, E4, L2, and L1 genes; X94165 Human papillomavirus type 73 E6, E7, E1, E2, E4, L2, and L1 genes; X67160 Human Papilloma Virus E6 gene for transforming protein; X70829 Protein, Human papillomavirus type 65 complete genome; X70828 Human papillomavirus type 63 complete genome; X70827 Human papillomavirus type 4 complete genome; X04354 Human papilloma virus (HPV-18) E6 ORF; X55965 Human papillomavirus type 57 complete DNA; X05568 Integrated HPV-18 E6/E7 ORF with DNaseI-hypersensitive site; X04773 Related Sequences, Human papilloma virus type 18 (HPV 18) DNA for early region with proteins E6, E7, E1X52061 Human papillomavirus (HPV) long control region; X52060 Human papillomavirus (HPV) long control region; X52059; Human papillomavirus (HPV) long control region; X52062 Human papillomavirus (HPV) long control region; X66269 HPV-16 enhancer sequence; U31794 Human papillomavirus type 66, complete genome; U31793 Human papillomavirus type 61, complete genome; U31792 Human papillomavirus type 60, complete genome; U31791 Human papillomavirus type 55, complete genome; U31790 Human papillomavirus type 50, complete genome; U31789 Human papillomavirus type 48, complete genome; U31788 Human papillomavirus type 44, complete genome; U31787 Human papillomavirus type 38, complete genome; U31786 Human papillomavirus type 37, complete genome; U31785 Human papillomavirus type 36, complete genome; U31784 Human papillomavirus type 29, complete genome; U31783 Human papillomavirus type 28, complete genome; U31782 Human papillomavirus type 24, complete genome; U31780 Human papillomavirus type 22, complete genome; U31779; Human papillomavirus type 21, complete genome; U31778 Human papillomavirus type 20, complete genome; J04351 Human papillomavirus type 11 (HPV-11) DNA; M 14710 Human papillomavirus type 18 (HPV 18) L1, (3′ end) and E6, (5′ end) genes; M72880 Human papillomavirus E6 protein (E6) gene sequence; M73458 Human papillomavirus E6 protein (E6) gene sequence; M73457 Human papillomavirus E6 protein (E6) gene sequence; M73456 Human papillomavirus E6 protein (E6) gene sequence; M80460 Human papillomavirus E6 protein (E6) gene sequence; J04353 Human papillomavirus type 31 (HPV-31) complete genome; K02718 Human papillomavirus type 16 (BPV16), complete genome; and M14119 Human papillomavirus type 11 (HPV-11) complete genome.

4. P19 Mouse Teratocarcinoma Cells

P19 cells have a number of characteristics that make them a suitable model for studying early mammalian development and especially development of the nervous system. The cells possess a normal karyotype (Mcburney, M. W. and Rogers, B. J. (1982) Dev. Biol. 89, 503-508), suggesting that they do not contain any gross genetic abnormalities, and stable transfected clonal sublines, which retain their ability to differentiate, are readily obtained with a high frequency. In addition, depending upon the treatment used, these multipotential P19 cells can differentiate into derivatives of all three germ layers: endoderm, mesoderm, and ectoderm (Bain, G. et al.. (1994) BioEssays 16, 343-348). These observations show that P19 cells are able to participate in many normal differentiation pathways. For example, treatment of aggregated P19 cells with retinoic acid results in cells that are capable of differentiating into neuron-, glia-, and fibroblast-like cells (Johnes-Villeneuve, E. M. V. et al. (1982) J. Cell. Biol. 94, 253-262).

5. Tetracycline Regulatory System

The tetracycline regulatory system can be used to study gene regulation in eukaryotes (Gossen, M. et al. (1993) Trends Biochem. Sci. 18, 471415). In this system, the Kruppel-associated box (KRAB) repressor domain of the human Kox 1 zinc finger protein (Margolin, J. et al. (1994) Proc. Natl. Acad. Sci. USA 91, 4509-4513) has been fused to a tetracycline repressor (tetR). In the absence of tetracycline, this chimeric DNA-binding protein (tetR-KRAB) exerts its silencing activity by binding to several cis-acting tetracycline operator (tetO) sites in front of a human cytomegalovirus (CMV) promoter. Alternatively, promoter activity is restored upon administration of tetracycline, preventing the binding of tetR-KRAB to tetO sequence. This system generates a very tight genetic switch (Deuschle, U. et al. (1995) Mol. Cell. Biol. 15, 1907-1914). Doxycycline was chosen as the inducer because it is one of the most potent effectors among the tetracycline derivatives (Gossen, M. et al. (1995) Science 268, 1766-1769). By u Gossen, M. et al. (1993) Trends Biochem. Sci. 18, 471-415sing the tetracycline-inducible system to control the expression of anti-sense TR4 during the RA-treatment in P19 cell lines, the function of TR4 in the process of differentiation of neuron cells was shown and disclosed herein. Using a tetracycline-inducible system to express antisense TR4, we demonstrated here that when this stable transfectant expressed anti-sense TR4 in the presence of doxycycline, there was a delayed response to RA both in the morphological and apoptotic responses consistent with TR4 being involved in RA-mediated apoptosis and differentiation.

6. Receptor DNA Recognition

Differential recognition of target genes by the steroid/thyroid hormone members is determined by at least three properties: protein-DNA interactions, protein-protein interactions, and protein environment. For the protein-DNA interaction, DNA-binding domains of family members selectively interact with HREs, which are structurally related but functionally distinct. Based on the zinc finger model, the proximal box in the DNA-binding domain of receptor proteins may determine target HRE specificity (Umesono, K., and Evans, R. M. (1989) Cell 57, 1139-1146). Consequently, the TR4 can be grouped into members of the estrogen receptor subfamily, recognizing direct repeats of the hexameric consensus motif AGGTCA (Umesono, K., and Evans, R. M. (1989) Cell 57, 1139-1146). Thus, it has been demonstrated that the TR4 may bind to tandem repeats, including the +55 region of the simian virus (SV) 40 major late promoter (Young, W.-J. et al. (1997) J. Biol. Chem. 272, 3109-3116), the fifth intron of the ciliary neurotrophic factor receptor (Lee, H.-J. et al. (1995) J. Biol. Chem. 270, 30129-30133), CRBPII and RARβ genes, respectively (Lee, Y.-F. et al. (1998) J. Biol. Chem. 273, 13437-13443). In addition, the TR4 has been shown to bind as a homodimer to direct repeats of the AGGTCA core sequence spaced by one (DR1) or more nucleotides (Hirose, T. et al. (1994) Mol. Endocrinol. 8, 1667-1680).

7. Steroid 21-hydroxylase

Steroid 21-hydroxylase (21-OHase; EC 1.14.99.10) belongs to the cytochrome P-450 superfamily, and is one of the key enzymes in biosynthesis of the steroid hormones, leading to the production of cortisol and aldosterone (Miller, W. L. (1988) Endocrine Rev. 9, 295-318). The expression of the 21-OHase gene is restricted to the adrenal cortex, where it is essential for the production of both glucocorticoids and mineralocorticoids. 21-OHase mediates the conversion of progesterone to 11-deoxycortisol. Since cortisol is the primary feedback inhibitor of corticotropin (ACTH) secretion, 21-OHase deficiency is one of the major causes of increasing levels of corticotropin causing congenital adrenal hyperplasia. This disorder is thus the most common autosomal recessive defect in humans (Miller, W. L. (1988) Endocrine Rev. 9, 295-318). The human 21-OHase gene has been cloned and characterized (Higashi, Y. et al. (1986) Proc. Natl. Acad. Sci. USA. 83, 2841-2845; White, P. C., et al. (1986) Proc. Natl. Acad. Sci. USA 83, 5111-5115; Rodrigues, N. R. et al. (1987) EMBO J. 6, 1653-1661; Speek, M., and Miller, W. L. (1995) Mol. Endocrinol. 9, 1655-1665). Subsequently, multiple regulatory elements have been identified from the 5′ flanking region of the 21-OHase gene, including the cyclic AMP-response element (John, M. E. et al. (1986) Proc. Natl. Acad. Sci. USA 83, 4715-4719; Handler, J. D. et al. (1988) J. Biol. Chem. 263, 13068-13073; Kagawa, N., and Waterman, M. R. (1990) J. Biol. Chem. 265, 11299-11305; Rice, D. A. et al. (1991) Mol. Endocrinol. 5, 1552-1561). In addition, members of the steroid receptor superfamily are suspected to regulate the expression of the adrenal steroidogenic enzymes (Rice, D. A. et al. (1991) Mol. Endocrinol. 5, 1552-1561). Indeed, it has been demonstrated that the NGFI-B/nur77/TR3 orphan receptor may bind to the mouse 21-OHase -65 element, and up-regulate this gene expression (Wilson, T. E. et al. (1993) Mol. Cell. Biol. 13, 861-868; Crawford, P. A. et al. (1995) Mol. Cell. Biol. 15, 4331-4336). The mouse 21-OHase -65 element does not exist in the 5′flanking region of the human gene. However, there is a perfect monomeric DNA response element containing the single AGGTCA core motif preceded by an AT-rich sequence located in the -228 element (nucleotide numbers 1431-1444, GenBank™ accession no. M12792 and M23280) in the human 21-OHase gene (White, P. C., et al. (1986) Proc. Natl. Acad. Sci. USA 83, 5111-5115)

It has been assumed that members of the steroid/thyroid hormone receptor family are able to bind to HREs in three different ways, monomeric, homodimeric, and heterodimeric (Glass, C. K. (1994) Endocrine Rev. 15, 391-407). Most classical steroid hormone receptors have been characterized to form homodimers or heterodimers with another member of the superfamily (Glass, C. K. (1994) Endocrine Rev. 15, 391-407). However, only a few members have sufficient affinity to bind to HREs as a monomer. Indeed, NGFI-B orphan receptor is the first nuclear protein for which binding to a yeast DNA element, referred to as the NGFI-B response element (NBRE), as a monomer was demonstrated (Wilson, T. E. et al. (1991) Science 252, 1296-1300; Wilson, T. E. et al. (1992) Science 256, 107-110). Two elements, referred to as the A and T boxes, at the C-terminal region of the second zinc finger of the NGFI-B/nur77/TR3 orphan receptor are critical for DNA recognition (Wilson, T. E. et al. (1991) Science 252, 1296-1300; Wilson, T. E. et al. (1992) Science 256, 107-110; Kurokawa, R. et al. (1993) Genes Dev. 7, 1423-1435). Several orphan receptors, for example, ROR/RZR, NGFI-B/nur77/TR3, FTZ-F1, and SF-1 may bind as monomers to HREs composed of a single AGGTCA motif preceded by an AT-rich sequence (Glass, C. K. (1994) Endocrine Rev. 15, 391-407; Wilson, T. E. et al. (1991) Science 252, 1296-1300; Wilson, T. E. et al. (1992) Science 256, 107-110; Ueda, H. et al. (1992) Mol. Cell. Biol. 12, 5667-5672; Giguere, V. et al. (1995) Mol. Cell. Biol. 15, 2517-2526; Schrader, M. et al. (1996) J. Biol. Chem. 271, 19732-19736). Thus, non-zinc finger regions of receptor proteins also contribute to the specificity of DNA binding. It was also demonstrated that the TR4 specifically binds to monomeric DNA response elements. The data disclosed herein demonstrate that the TR4 can function as a repressor in human steroid 21-hydroxylase gene regulation via the −228TR4RE. Thus, the 21-OHase gene represents a unique target down-regulated by the TR4 orphan receptor.

8. PPAR Pathways

A PPARα signal pathway can enhance keratinocyte differentiation (Hanley, K et al. (1998) J Invest Dermatol 110, 368-375). Disclosed herein is the cross-talk between TR2/TR4 and PPARα signaling pathways in HaCaT cells. Disclosed herein TR2, as well as TR4, suppresses RA-mediated, but not vitamin D-mediated, transcriptional activity in human HaCaT keratinocytes, using a chloramphenicol acetyltransferase (CAT) reporter gene assay. Also disclosed herein, TR4, but not TR2 can significantly suppress the Wy-14643-induced PPARα transactivation in HaCaT keratinocytes. Furthermore, Wy-14643 can increase TR4 and decrease TR2 in HaCaT keratinocytes. These results indicate that a differential bi-directional regulation between TR2/TR4 and PPARα can occur in HaCaT keratinocytes, providing the evidence that TR2 and TR4 may play important roles in HaCaT keratinocytes.

9. Molecules That Inhibit TR2/TR4 DNA Binding Interactions

Disclosed are compositions that inhibit TR2 and TR4 effects on the pathways disclosed herein. Also disclosed are compositions and methods for the isolation of molecules that can modulate, such as inhibit or increase the effects on the pathways disclosed herein. Some of these molecules for example, can be functional nucleic acids, antibodies, peptide mimetics or other small molecules.

a) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of genes disclosed herein or the genomic DNA of genes disclosed herein or they can interact with the polypeptide of the genes disclosed herein. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than 10⁻⁶. It is more preferred that antisense molecules bind with a k_{d} less than 10⁻⁸. It is also more preferred that the antisense molecules bind the target molecule with a k_{d} less than 10⁻¹⁰. It is also preferred that the antisense molecules bind the target molecule with a k_{d} less than 10⁻¹². A representative sample of methods and techniques which aid in the design and use of antisense molecules can be found in the following non-limiting list of U.S. Pat. Nos.: 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶. It is more preferred that the aptamers bind the target molecule with a k_{d} less than 10⁻⁸. It is also more preferred that the aptamers bind the target molecule with a k_{d} less than 10⁻¹⁰. It is also preferred that the aptamers bind the target molecule with a k_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10 fold lower than the k_{d} with a background binding molecule. It is more preferred that the aptamer have a k_{d} with the target molecule at least 100 fold lower than the k_{d} with a background binding molecule. It is more preferred that the aptamer have a k_{d} with the target molecule at least 1000 fold lower than the k_{d} with a background binding molecule. It is preferred that the aptamer have a k_{d} with the target molecule at least 10000 fold lower than the k_{d} with a background binding molecule. It is preferred when doing the comparison for a polypeptide for example, that the background molecule be a different polypeptide. For example, when determining the specificity of disclosed aptamers, the background protein could be serum albumin. Representative examples of how to make and use aptamers to bind a variety of different target molecules can be found in the following non-limiting list of U.S. Pat. Nos.: 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos.: 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos.: 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos.: 5,595,873 and 5,652,107). There are also a number of ribozymes that are not found in natural systems, but which have been engineered to catalyze specific reactions de novo (for example, but not limited to the following U.S. Pat. Nos.: 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos.: 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶. It is more preferred that the triplex forming molecules bind with a k_{d} less than 10⁻⁸. It is also more preferred that the triplex forming molecules bind the target molecule with a k_{d} less than 10⁻¹⁰. It is also preferred that the triplex forming molecules bind the target molecule with a k_{d} less than 10⁻¹². Representative examples of how to make and use triplex forming molecules to bind a variety of different target molecules can be found in the following non-limiting list of U.S. Pat. Nos.: 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos.: 5,168,053, 5,624,824, 5,683,873, 5,728,521, 5,869,248, and 5,877,162

b) Antibodies

### (1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with the disclosed compositions such that the disclosed effects of TR2 and TR4 are modulated, such as inhibited or increased. Antibody also includes, chimeric antibodies and hybrid antibodies, with dual or multiple antigen or epitope specificities, and fragments, such as F(ab′)2, Fab′, Fab and the like, including hybrid fragments, as well as conjugates of antibody fragments and antigen binding proteins (single chain antibodies) as described, for example, in U.S. Pat. No. 4,704,692, the contents of which are hereby incorporated by reference. The antibodies can be tested for their desired activity using the in vitro assays described herein, or by analogous methods, after which their in vivo therapeutic and/or prophylactic activities are tested according to known clinical testing methods. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, monoclonal antibodies of the invention can be prepared using hybridoma methods, such as those described by Kohler and Mulstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the binding domains of the compositions described, herein, such as the PTAP binding domain, described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis or a specific region of the protein, followed by expression and testing of the expressed polypeptide. Such methods are readily apparent to a skilled practitioner in the art and can include site-specific mutagenesis of the nucleic acid encoding the antibody or antibody fragment (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods of the invention serves to lessen the chance that an antibody administered to a human will evoke an undesirable immune response.

### (2) Human Antibodies

The human antibodies of the invention can be prepared using any technique.

Examples of techniques for human monoclonal antibody production include those described by Cole et al. (Monoclonal Antibodies and Cancer Therapy, Alan R. Liss, p.77, 1985) and by Boerner et al. (J. Immunol., 147(1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No.5,721,367 (Kay et al.), U.S. Pat. No. 5,837,243 (Deo et al.), U.S. Pat. No.5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti TSG101 antibodies and antibody fragments of the invention can also be administered to patients or subjects as a nucleic acid preparation (e.g., DNA or RNA) that encodes the antibody or antibody fragment, such that the patient′s or subject′s own cells take up the nucleic acid and produce and secrete the encoded antibody or antibody fragment. The delivery of the nucleic acid can be by any means, as disclosed herein, for example.

c) Compositions Identified by Screening with Disclosed Compositions/Combinatorial Chemistry

### (1) Combinatorial Chemistry

The disclosed compositions can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target molecule′s function. The molecules identified and isolated when using the disclosed compositions, such as, TR2 and TR4, are also disclosed. Thus, the products produced using the combinatorial or screening approaches that involve the disclosed compositions, such as, TR2 and TR4, are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak I. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain. A peptide of choice is attached to a DNA binding domain of a transcriptional activation protein, such as Gal 4. By performing the Two-hybrid technique on this type of system, molecules that bind the peptide of choice can be identified.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5 5,856,496), imidazol-pyrido-indole and imidazol-pyrido-benzothiophenes (U.S. Pat. No. 5,856,107) substituted 2-methylene-2, 3-dihydrothiazoles (U.S. Pat. No. 5,847,150), quinolines (U.S. Pat. No. 5,840,500), PNA U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in interative processes.

### (2) Computer Assisted Drug Design

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties such as inhibition or stimulation or the target molecule′s function. The molecules identified and isolated when using the disclosed compositions are also disclosed. Thus, the products produced using the molecular modeling approaches that involve the disclosed compositions are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, OSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

10. Aspects Applicable to All Compositions

a) Sequence similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

b) Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6× SSC or 6× SSPE) at a temperature that is about 12-25° C. below the Tm (the melting temperature at which half of the molecules dissociate from their hybridization partners) followed by washing at a combination of temperature and salt concentration chosen so that the washing temperature is about 50° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6× SSC or 6× SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

c) Nucleic Acids

There are a variety of molecules disclosed herein that are nucleic acid based, including for example the nucleic acids that encode, for example TR2, TR4, RAR, RXR, PPAR, 21-Ohase, and the HPV genome as well as any other protein disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### (1) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### (2) Sequences

There are a variety of sequences related to, for example TR2, TR4, RAR, RXR, PPAR, 21-Ohase, and the HPV genome as well as any other protein disclosed herein that are disclosed on Genbank, and these sequences and others are herein incorporated by reference in their entireties as well as for individual subsequences contained therein.

Sequences related to the human 21-OHase gene and having GenBank™ accession no. M12792 and M23280 are used herein, as an example, to exemplify the disclosed compositions and methods. It is understood that the description related to this sequence is applicable to any sequence related to human 21-OHase or any other disclosed protein unless specifically indicated otherwise. Those of skill in the art understand how to resolve sequence discrepancies and differences and to adjust the compositions and methods relating to a particular sequence to other related sequences (i.e. sequences of 21-OHase). Primers and/or probes can be designed for any 21-OHase sequence given the information disclosed herein and known in the art.

### (3) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

d) Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991)Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### (1) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as TR2 or TR4 into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone, as well as lentiviruses. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is′inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

(a) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

(b) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462477 (1970); Brown and Burlingham, J. Virology 12:386-396 (1973); Svensson and Persson, J. Virology 55:442449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virions are generated in a cell line such as the human 293 cell line. In another preferred embodiment both the E1 and E3 genes are removed from the adenovirus genome.

(c) Adeno-associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (TTRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRS, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

(d) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 3341, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

### (2) Non-nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed molecules or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No.4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta. 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### (3) In vivo/ex vivo

As described above, the compositions can be administered in a pharmaceutically acceptable carrier and can be delivered to the subject=s cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

e) Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (1) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### (2) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

f) Peptides

### (1) Protein Variants

As discussed herein there are numerous variants of the disclosed proteins that are known and herein contemplated. In addition, to the known functional strain variants there are derivatives of the disclosed proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-inking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | Ala; A |
| allosoleucine | AIle |
| arginine | Arg; R |
| asparagine | Asn; N |
| aspartic acid | Asp; D |
| cysteine | Cys; C |
| glutamic acid | Glu; E |
| glutamine | Gln; Q |
| glycine | Gly; G |
| histidine | His; H |
| isolelucine | Ile; I |
| leucine | Leu; L |
| lysine | Lys; K |
| phenylalanine | Phe; F |
| proline | Pro; P |
| pyroglutamic acidp | Glu |
| serine | Ser; S |
| threonine | Thr; T |
| tyrosine | Tyr; Y |
| tryptophan | Trp; W |
| valine | Val; V |

TABLE 2
Amino Acid Substitutions
Original Residue Exemplary Conservative Substitutions,
others are known in the art.

| | |
|---|---|
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular organism from which that protein arises is also known and herein disclosed and described.

g) Pharmaceutical Carriers/Delivery of Pharamceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions maybe administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No.3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother.. 35:421425, (1992); Pietersz and McKenzie, Immunolog. Reviews. 129:57-80, (1992); and Roffler, et al., Biochem. Pharmacol 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### (1) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer′s solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable..

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### (2) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, N.Y. (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing an HBV infection, the efficacy of the therapeutic antibody can be assessed in various ways well known to the skilled practitioner. For instance, one of ordinary skill in the art will understand that a composition, such as an antibody, disclosed herein is efficacious in treating or inhibiting an HBV infection in a subject by observing that the composition reduces viral load or prevents a further increase in HBV viral load. Viral loads can be measured by methods that are known in the art, for example, using polymerase chain reaction assays to detect the presence of HBV nucleic acid or antibody assays to detect the presence of HBV protein in a sample (e.g., but not limited to, blood) from a subject or patient, or by measuring the level of circulating anti-HBV antibody levels in the patient.

Other molecules that modulate TR2 or TR4 function as disclosed herein which do not have a specific pharmacuetical function, but which may be used for tracking changes within a cellular environment.

The disclosed compositions and methods can also be used for example as tools to isolate and test new drug candidates for a variety of TR2 and TR4 related diseases.

h) Chips and Micro Arrays

Disclosed are chips where at least one address is the sequences or part of the sequences set forth in any of the nucleic acid sequences disclosed herein. Also disclosed are chips where at least one address is the sequences or portion of sequences set forth in any of the peptide sequences disclosed herein.

Also disclosed are chips where at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

i) Computer Readable Mediums

It is understood that the disclosed nucleic acids and proteins can be represented as a sequence consisting of the nucleotides of amino acids. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer readable mediums, such as, commercially available floppy disks, tapes, chips, hard drives, compact disks, and video disks, or other computer readable mediums. Also disclosed are the binary code representations of the disclosed sequences. Those of skill in the art understand what computer readable mediums. Thus, computer readable mediums on which the nucleic acids or protein sequences are recorded, stored, or saved.

Disclosed are computer readable mediums comprising the sequences and information regarding the sequences set forth herein.

11. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include primers to perform the amplification reactions discussed in certain embodiments of the methods, as well as the buffers and enzymes required to use the primers as intended.

D. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W. H. Freeman and Co., N.Y. (1992); Bodansky M and Trost B., Ed. (1993) Principles of Peptide Synthesis. Springer-Verlag Inc., N.Y. (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton RC et al., Techniques in Protein Chemistry IV. Academic Press, N.Y., pp. 257-267 (1992)).

3. Process Claims for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed

peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate including a human, ape, monkey, orangutang, or chimpanzee.

Also disclosed are animals produced by the process of adding to the animal any of the cells disclosed herein.

E. Methods of Using the Compositions

1. Methods of Using the Compositions as Research Tools

The compositions can be used for example as targets in combinatorial chemistry protocols or other screening protocols to isolate molecules that possess desired functional properties related to TR2 and TR4 DNA binding, for example.

The disclosed compositions can also be used diagnostic tools related to diseases such as HPV related diseases or TR2/TR4 related diseases.

The disclosed compositions can be used as discussed herein as either reagents in micro arrays or as reagents to probe or analyze existing microarrays. The disclosed compositions can be used in any known method for isolating or identifying single nucleotide polymorphisms. The compositions can also be used in any known method of screening assays, related to chip/micro arrays. The compositions can also be used in any known way of using the computer readable embodiments of the disclosed compositions, for example, to study relatedness or to perform molecular modeling analysis related to the disclosed compositions.

2. Methods of Gene Modification and Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a cell is transformed with a vector which is designed to homologously recombine with a region of a particular chromosome contained within the cell, as for example, described herein. This homologous recombination event can produce a chromosome which has exogenous DNA introduced, for example in frame, with the surrounding DNA. This type of protocol allows for very specific mutations, such as point mutations, to be introduced into the genome contained within the cell. Methods for performing this type of homologous recombination are disclosed herein.

One of the preferred characteristics of performing homologous recombination in mammalian cells is that the cells should be able to be cultured, because the desired recombination event occur at a low frequency.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

3. Method of Treating Cancer

The disclosed compositions can be used to treat any disease where uncontrolled cellular proliferation occurs such as cancers. Disclosed are methods for inhibiting cancers related to TR2 or TR4 related cancers for example, as they relate to, for example, to HPV related cancers and retinoid related cancers, for example. HPV related cancers include precancer conditions such as cervical and anal dysplasias, other dysplasias, severe dysplasias, hyperplasias, atypical hyperplasias, and neoplasias. Other related HPV cancers include genital and vaginal cancers, as well as oral cancers. For example CIN1-CIN3. Also disclosed are methods for inhibiting cancers that are related to p53 and Rb.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### F. EXAMPLS

### 1. Example 1

### Feedback Regulation Between Orphan Nuclear Receptor TR2 and Human Papilloma Virus Type 16

The human TR2 orphan receptor (TR2), initially isolated from testis and prostate cDNA libraries, is a member of the steroid receptor superfamily. TR2 can regulate several target genes via binding to a consensus response element (AGGTCA) in direct-repeat orientation (AGGTCAX(n)AGGTCA, n=0-6). Here it is shown that TR2 is able to induce the expression of human papillomavirus type-16 (HPV-16) genes via binding to a DR4 response element n the long control region of HPV-16. Additionally, one of the HPV-16 gene products, the E6 oncogene, regulates TR2 gene expression. A likely mechanism for this regulation involves E6-mediated degradation of the tumor suppressor p53, a protein known to suppress TR2 expression. Together, the data disclosed herein provide evidence for feedback regulation between TR2 and HPV-16, which represents a regulatory pathway involving a member of the steroid receptor superfamily and the HPV-DNA tumor virus.

a) Materials and Methods

### (1) Immunohistochemical Staining

Three 8-10 week old female ICR mice (Taconic, Germantown, N.Y.) were sacrificed, and the vagina, uterus, and cervix of each animal were removed. The tissue was fixed in Histochoice MB (Amresco, Solon, Ohio) at 4° C. overnight, and embedded in paraffin. Saggital sections through each of the three anatomic regions of interest were cut at a thickness of 7 μm, dried at 37° C. overnight, and processed for modified hemotoxylin/eosin (Culling, C. F. A. (1963) Handbook of Histopathological Techniques Butterworths, Inc.,

Washington) and immunohistochemical staining. For immunohistochemical staining, sections were stained with the mouse monoclonal, anti-TR2 antibody G204, and a biotinylated anti-mouse secondary antibody (Vector Laboratories, Burlingame, Calif.). Staining was visualized upon development with the DAB substrate kit (Vector Laboratories, Burlingame, Calif.). Stained sections were analyzed via light microscopy (Nikon, Tokyo, Japan) and photographed.

### (2) Plasmids

The TR2pCAT reporter plasmid consists of the 2.7 kb 5′-flanking region of the TR2 orphan receptor (Lin, D. et al. (1998) Endocrine 8, 123-134) linked to the CAT gene. pC53-SN3 (gift from B. Vogelstein, described in Baker, S. J. et al. (1990) Science 249, 912-915) was constructed by inserting wild-type p53 cDNA into the pCMV-Neo-Bam vector. p1321 and p1434 (gifts from P. M. Howley, described in Munger, K., et al. (1989) J. Virology 63, 4417-4421) contain the E6 and E7 genes of HPV-16, driven by the human β-actin promoter. p1321 expresses both E6 and E7 genes, whereas p1434 contains a translation termination linker within E6. The plasmid pSG5-hTR2-11 (Lee, H. et al. (1993) Biochem. Biophys. Res. Commun. 194, 97-103) expresses full-length TR2, and the plasmid pCMX-TR4 (gift from R. M. Evans, described in Lee, H. et al. (1995) J. Biol. Chem. 270, 30129-30133) expresses full length TR4. For coupled in vitro transcription and translation, pSG5-hTR2-11 (Lee, H. et al. (1993) Biochem. Biophys. Res. Commun. 194, 97-103) was used. The HPV-16 LCR-CAT reporter plasmid was constructed using EcoRV-Hind III digested pBSCAT2 as a vector into which a 600 bp Sma I-Hind III fragment released from pGL2-LCR (wt) (gift from G. J. Sibbet, described in Sibbet, G. J. et al. (1995) J. Virology 69, 4006-4011) was inserted. Dideoxy chain termination DNA sequencing (Sanger, F. et al. (1977) Proc. Natl. Acad. Sci. USA 74, 5463-5467) of HPV-16 LCR-CAT was conducted to confirm the integrity of the plasmid sequence. pSG5-TR2-AR-TR2 (p2A2) was constructed by replacing the DNA-binding domain of TR2 with that of the androgen receptor (AR) in the context of the pSG5 expression vector, employing a method similar to that used to create the chimeric pSG5-AR-TR2-AR plasmid (Lee, H. et al. (1993) Biochem. Biophys. Res. Commun. 194, 97-103). Site-directed mutagenesis (Lee, H. et al. (1993) Biochem. Biophys. Res. Commun. 194, 97-103) was utilized to produce HPV-16 LCR-D27CAT, which lacks a 27 bp region of the HPV-16 LCR, including the HPV-16 DR4RE. The plasmids HPV-DR4RE(+)-TKCAT and HPV-DR4RE(−)-TKCAT were constructed by insertion of the 16 bp HPV-16 DR4RE oligonucleotide into the −32TK-CAT vector (gift from R. C. Ralff (Ralff, C. et al., JBC 276:27316-27321 (2001) in either sense(+) or antisense(−) orientation. The pCMVβgal expression vector was co-transfected in all CAT assay experiments to enable normalization of transfection efficiency (Lee, H. et al. (1993) Biochem. Biophys. Res. Commun. 194, 97-103).

### (3) Cell Culture, Transfection, and CAT Assay

H1299 cells were cultured as previously described (Lin, D., and Chang, C. (1996) J. Biol. Chem. 271, 14649-14652). Chinese Hamster Ovary (CHO) cells were cultured in Dulbecco's Modified Eagle's F-12 medium supplemented with 5% fetal bovine serum, 100 μg/ml penicillin, 100 μg/ml streptomycin, and 0.25 μg/ml amphotericin (Sigma, St. Louis, Mo.). Twenty-four hours before transfection, cells were plated at a density of 10₆ cells per 60 mm tissue culture plate. Cells were transfected with a total of 10.5 μg plasmid DNA per plate via the calcium phoshpate method (Chen, C., and Okayama, H. (1987) Mol. Cel. Biol. 7, 2745-2752). Specific plasmids and doses used are described in Figures and associated Figure Legends. Twenty-four hours after transfection, the medium was changed. After another 24 hours, cells were subjected to freeze-thaw lysis in 250 mM Tris-HCl (pH 7.8), and the resulting cell extracts were assayed for chloramphenicol acetyltransferase (CAT) activity. CAT activity was assessed via Phosphorimager scanning and quantitated using ImageQuant software (Molecular Dynamics Inc., Sunnyvale, Calif.).

### (4) In Vitro Transcription/Translation

The expression vector pSG5-hTR2-11 was utilized to produce in vitro transcribed and translated TR2 protein in the TNT coupled rabbit reticulocyte lysate system (Promega, Madison, Wis.).

### (5) Electophoretic Mobility Shift Assay (EMSA)

EMSA analysis was performed as described previously (Lee, H. J., and Chang, C. (1995) J. Biol. Chem. 270, 5434-5440). Briefly, the HPV-16 DR4RE oligonucleotide was end-labeled with ₃₂P to serve as a probe to demonstrate the binding of in vitro expressed TR2 to the DR4 sequence 8 within the LCR of HPV-16. For competition reactions, unlabeled HPV-16 DR4RE oligonucleotides were mixed with labeled probe prior to its addition to the reaction. Antibody shift analysis involved the addition of 1 μl of the monoclonal anti-TR2 orphan receptor antibody, G204, to the reaction for 15 min., at room temperature, prior to gel loading.

b) Results

### (1) TR2 Protein is Expressed in Cell Types Commonly Infected by HPV-16

To establish physiological relevance for studying the interaction of regulatory pathways involving TR2 and HPV-16, staining of mouse vaginal and cervical tissue, with a monoclonal antibody specific to TR2 (G204), was performed. As HPV-16 is epitheliotropic, and the genital form commonly infects the stratified squamous epithelial layers of the uterine cervix and vagina (Bosch, F. X. et al. (1995) J. Natl. Cancer Inst. 87, 796-802), it was determined whether TR2 was expressed in the same cell types within these regions. The anti-TR2 monoclonal antibody G204 was used in immunohistochemical staining of longitudinal sections of paraffin-embedded mouse cervix and vagina samples. After staining, it was clear that TR2 was present in the nuclei of stratified squamous epithelial cells within the regions known to be susceptible to BPV-16 infection (FIG. 1).

### (2) HPV-16 Oncogene E6 Antagonizes p53-mediated Repression of TR2 Gene Expression

TR2 levels in MCF-7 cells are repressed by ionizing radiation, and that the kinetics of the down regulation of TR2 correlated with a concurrent increase in p53 expression (Lin, D., and Chang, C. (1996) J. Biol. Chem. 271, 14649-14652). Additionally, it was shown that upon co-transfection of the SV40 large T antigen, a viral oncoprotein known to associate with, and inactivate p53 (Lane, D. P. and Crawford, L. V. (1979) Nature 278, 261-263; Linzer, D. I. H. and Levine A. J. (1979) Cell 17, 43-52; Levine, A. J. et al. (1994) Br. J. Cancer 69, 409-416; Mietz, J. A. et al. (1992) EMBO J. 11, 5013-5020), TR2 reporter expression was not repressed after exposure of cells to ionizing radiation. Further, using expression plasmids expressing either wildtype or mutant forms of p53, it was found that wildtype p53 was able to mediate suppression of TR2 expression. The mutant form of p53 was unable to affect expression of TR2. Having demonstrated repression of TR2 mediated by the p53 tumor suppressor (Lin, D., and Chang, C. (1996) J. Biol. Chem. 271, 14649-14652), and recognizing that HPV-16 E6 binds and promotes degradation of p53 (Werness, B. A. et al. (1990) Science 248, 76-79; Scheffner, M. et al. (1990) Cell 63, 1129-1136), it was determined whether the suppressive effect of p53 on TR2 expression could be attenuated by E6. Using the lung-derived, p53-null H1299 cell line, the effect of increasing levels of a plasmid expressing both the HPV-16 E6 and E7 proteins on TR2 promoter activity was tested (FIG. 2). To demonstrate the suppressive effect of p53 on TR2-reporter gene (TR2pCAT) (22) expression, TR2pCAT and an expression plasmid containing p53 (23) were co-transfected. This resulted in a 4-fold suppression of TR2-reporter gene expression (lane 2), compared with reporter expression after transfection of the reporter alone (lane 1). The suppressive effect was attenuated in a dose dependent manner upon the addition of the p1321 expression plasmid (Munger, K., et al. (1989) J. Virology 63, 4417-4421), which expresses both the HPV-16 E6 and E7 proteins (lanes 3 and 4). A plasmid containing a translation termination linker (TTL) early in the E6 ORF (p1434) (Munger, K., et al. (1989) J. Virology 63, 4417-4421), that expresses a truncated version of E6 along with full length E7, was unable to efficiently attenuate the suppressive effect of p53 (lane 5 vs. lanes 3 and 4). These data suggest that the HPV-16 E6 oncogene, through its role in targeting p53 for ubiquitin-mediated degradation, is able to significantly reduce p53 suppression of TR2. As amino acids 106-115 of the C-terminus of HPV-16 E6 are necessary for efficient association with p53 (Crook, T. et al. (1991) Cell 67, 547-556), it is unlikely that the 52 amino acid, truncated form of E6, expressed from plasmid p1434 (Munger, K., et al. (1989) J. Virology 63, 4417-4421), is able to bind p53. It has also been shown that the ability of E6 to bind p53 is necessary for p53 degradation (Crook, T. et al. (1991) Cell 67, 547-556), which may explain the loss of the attenuating effect of truncated E6 on p53-mediated suppression of TR2-promoter expression.

### (3) Identification of a Direct Repeat 4 Response Element in the HPV-16 LCR

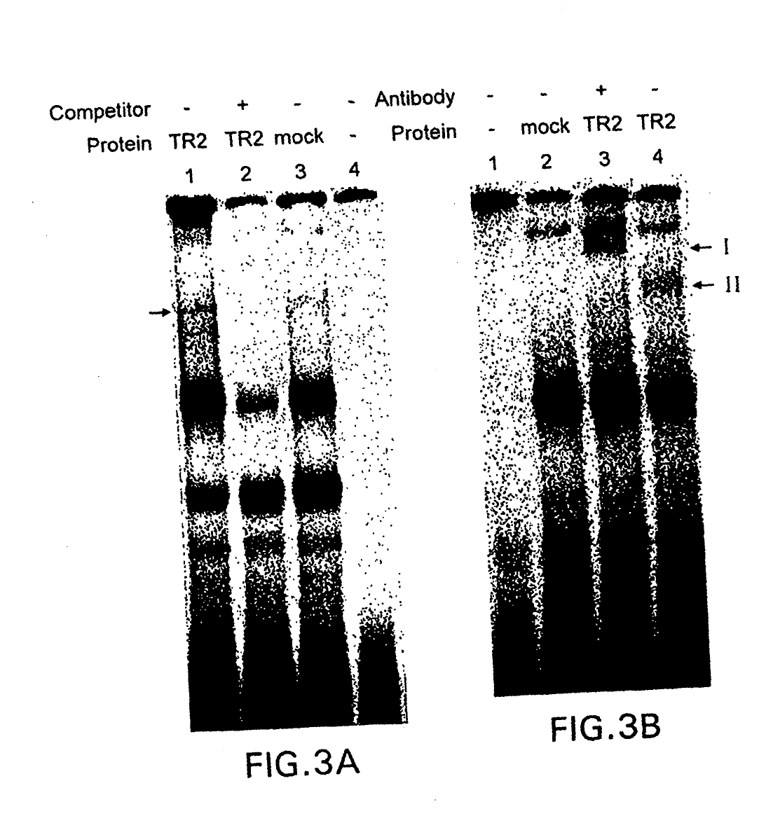

Sequence analysis of the HPV-16 LCR resulted in identification of a potential TR2 response element located 175 bp upstream of the TATA-box at bp 65 of the E6 ORF. The site consists of two consensus A(G/T)(G/T)TCA half sites with a spacing of 4 nucleotides (ATGTCAccctAGTTCA). To determine whether TR2 was able to bind the HPV-16 DR4RE, gel shift analysis was performed. FIG. 3A demonstrates that in vitro transcribed and translated Th2 protein is able to form a complex with ³²P-labeled HPV-16 DR4RE oligonucleotides (lane 1, arrow indicates complex), and that a 100-fold excess of unlabeled HPV-16 DR4RE is able to eliminate formation of the labeled complex (lane 2). Also in FIG. 3A, a mock in vitro transcription/translation reaction was used in lane 3, and lane 4 contains ³²P-labeled HPV-16 DR4RE alone. To further confirm the binding specificity of TR2 for the HPV-16 DR4RE, a TR2-specific monoclonal antibody was added, resulting in an upward shift of the labeled complex (FIG. 3B). FIG. 3B, lane 1 shows ₃₂P-labeled HPV-16 DR4RE alone, and lane 2 contains the product of a mock in vitro transcription/translation reaction. FIG. 3B, lane 4, demonstrates complex formation between TR2 and ³²P-labeled HPV-16 DR4RE (arrow II), while addition of a TR2-specific antibody in lane 3 resulted in slower migration of the labeled complex (arrow I). These data provide evidence for the existence of a TR2 response element within the HPV-16 LCR.

### (4) Induction of HPV-16 LCR-directed Gene Expression by TR2

Having determined that E6 is able to affect TR2 expression (FIG. 2), presumably through induction of p53 degradation, and after discovering a TR2 response element within the HPV-16 LCR (FIG. 3), whether TR2 could regulate the expression of the E6 oncogene was shown. An expression vector containing the region which controls E6 gene expression (pGL2-LCR, containing the HPV-16 LCR) (Sibbet, G. J. et al. (1995) J. Virology 69, 4006-4011) was linked to a CAT reporter (HPV-16 LCR-CAT), and then co-transfected with a vector constructed to express TR2 (pSG5-hTR2-11) (Lee, H. et al. (1993) Biochem. Biophys. Res. Commun. 194, 97-103), in the Chinese hamster ovary (CHO) cell line. As shown in FIG. 4, the HPV-16-LCR-driven CAT expression was significantly induced by TR2 in a dose-dependent manner (lanes 1-5). A similar induction of HPV-16 LCR-CAT activity also occurred when the TR2-expressing plasmid was replaced with that which expresses the highly homologous TR4 orphan receptor (FIG. 4, lanes 10-13) (Chang, C. et al. (1994) Proc. Natl. Acad. Sci. USA 91, 6040-6044). Both TR2 and TR4 recognize the same consensus HRE sequence and are able to modulate some of the same target genes (Young, W. et al. (1998) J. Biol. Chem. 273, 20877-20885; Lee, H. J., and Chang, C. (1995) J. Biol. Chem. 270, 5434-5440; Lee, H. et al. (1995) J. Biol. Chem. 270, 30129-30133; Lee, Y. et al. (1998) J. Biol. Chem. 273, 13437-13443). Therefore, it is not surprising that both TR2 and TR4 are able to induce HPV-16 LCR-CAT activity. In contrast, when we replaced pSG5-hTR2-11 with pSG5-TR2-AR-TR2 (2A2) (Lee, H. et al. (1993) Biochem. Biophys. Res. Commun. 194, 97-103), which expresses a chimeric receptor in which the DBD of the androgen receptor is substituted for the TR2-DBD, the induction of HPV-16 LCR-CAT was lost (FIG. 4, lanes 6-9). This loss of reporter induction suggests that the DBD of TR2 is essential for recognition of the HRE located within the HPV-16 LCR.

### (5) Functional Analysis of the RPV-16 DR4RE

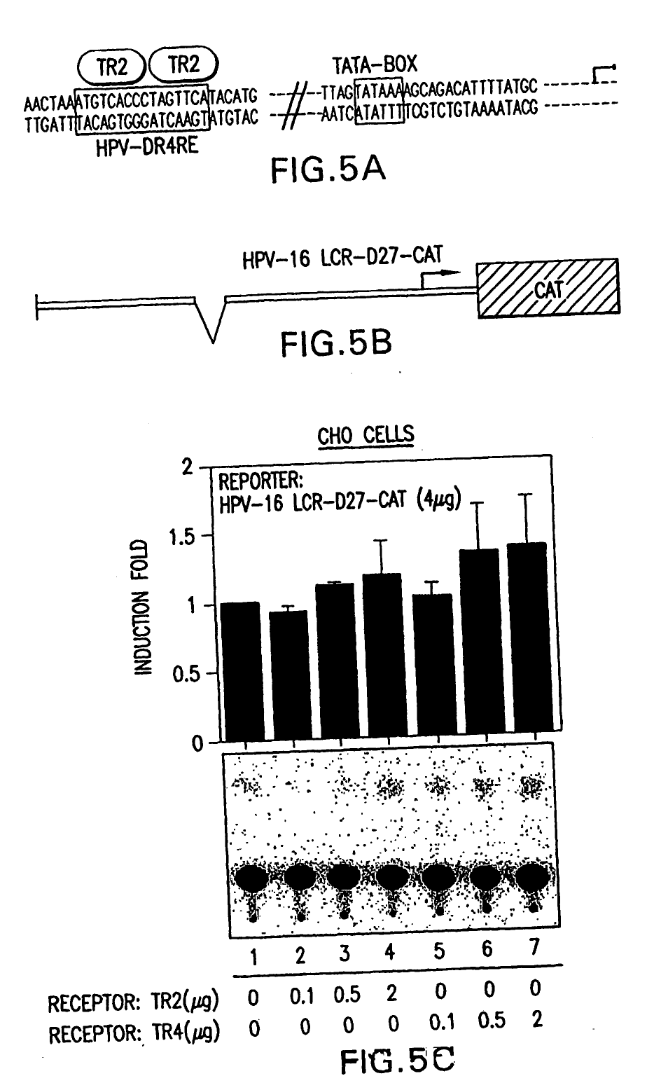

To determine whether the identified HPV-16 DR4RE is necessary for the regulatory effect of TR2 on E6 expression, a site-directed PCR mutagenesis strategy (Lee, H. et al. (1993) Biochem. Biophys. Res. Commun. 194, 97-103) was used to create a mutant of HPV-16 LCR-CAT. This mutant of the region responsible for regulation of HPV gene expression (HPV-16 LCR-CAT) has an internal deletion of 27 bp, including the HPV-16 DR4RE, the sequence recognized by TR2 (FIG. 4A and 4B). The mutant (HPV-16 LCR-D27CAT) was co-transfected with expression vectors that contain either TR2 or TR4 (Lee, H. et al. (1993) Biochem. Biophys. Res. Commun. 194, 97-103; Lee, H. et al. (1995) J. Biol. Chem. 270, 30129-30133), into CHO cells. In contrast to HPV-16 LCR-CAT, which can be significantly induced by TR2 or TR4 (FIG. 4), the CAT activity of HPV-16-LCR-D27CAT showed no significant increase with transfection of increasing concentrations of TR2 (FIG. 5, lanes 2-4), or TR4 (FIG. 5, lanes 5-7) expression plasmids. These data demonstrate that the HPV-DR4RE, located 175 bp upstream of the TATA-box at bp 65 in the E6 ORF, is necessary for TR2-mediated induction of HPV-16 gene expression.

### (6) Identification of the HPV-16 DR4RE as an Enhancer for Induction of the Thymidine Kinase (TK) Promoter

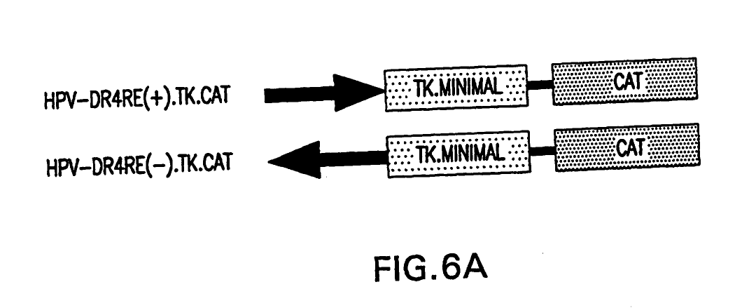

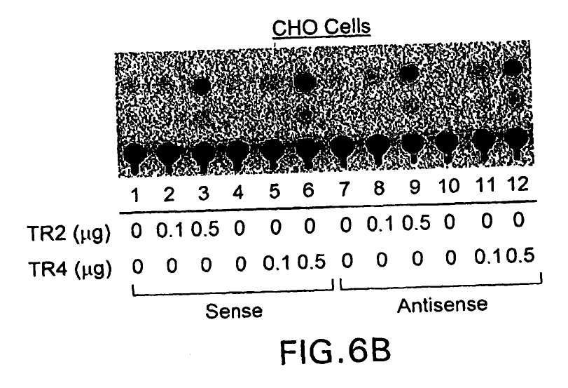

To determine whether the TR2 response element sequence present in the LCR of HPV-16 is sufficient to allow TR2-mediated regulation of HPV-16 early gene expression, the response element sequence (HPV-16 DR4RE) was tested outside of the context of the HPV-16 LCR. The same unlabeled oligonucleotide (HPV-16 DR4RE) that was used in the gel shift assay (FIG. 2) was inserted into the −32TK-CAT vector, in either sense or antisense orientation (FIG. 6A). CHO cells were then transfected with either sense HPV-DR4RE (+)TK-CAT or antisense HPV-DR4RE(−) TK-CAT, along with increasing amounts of TR2 or TR4. Our data exhibit induction of reporter activity by both TR2 (FIG. 6B, lanes 1-3 and 7-9) and TR4 (FIG. 6B, lanes 4-6 and 10-12) in a dose-dependent manner, regardless of orientation. These data suggest that the HPV-16 DR4RE alone is sufficient to allow TR2 or TR4-mediated upregulation of viral gene promoter expression, as demonstrated by the induction of transcriptional activity of the TK-minimal promoter by these receptors.

### 2. Example 2

### Repression of the Human Steroid 21-hydroxylase Promoter by the TR4 Orphan Receptor is Mediated by the Monomeric AGGTCA Motif

a) Materials and Methods

### (1) Plasmid Construction

Plasmid pCMX-TR4 consists of the full-length coding sequence of the TR4 cDNA in the pCMX vector under the control of both the human cytomegalovirus and T7 promoters for in vivo and in vitro expression, respectively (Lee, H.-J. et al. (1995) J. Biol. Chem. 270, 30129-30133). Plasmids 100CAT21 and 389CAT21 containing different lengths of the natural promoter regions of the 21-OHase gene in front of the chloramphenicol acetyltransferase (CAT) reporter gene were kindly provided by Dr. Michael R. Waterman as described previously (Kagawa, N., and Waterman, M. R. (1990) J. Biol. Chem. 265, 11299-11305). Plasmids pCMX-RXRα and pT7hmSHP were kindly provided by Drs. Ronald M. Evans and Wongi Seol, respectively (Lee, H.-J. et al. (1995) J. Biol. Chem. 270, 30129-30133, Lee, H.-J. et al. (1996) J. Biol. Chem. 271, 10405-10412). One copy of the −228TR4RE (nucleotide numbers 1431-1444, 5′-GGAAAAAGGTCAGG-3′) of the human 21-OHase gene (White, P. C., et al. (1986) Proc. Natl. Acad. Sci. USA 83, 5111-5115) was cloned into the Bgl II site of the pGL3-promoter vector (Promega), named pGL3-228.

### (2) Coupled In Vitro Transcription and Translation

Plasmids containing the full-length TR4 orphan receptor, RXRα, and SHP cDNAs were in vitro transcribed and translated directly by the TNT system (Promega) as previously described (Lee, H.-J. et al. (1995) J. Biol. Chem. 270, 30129-30133). Depending on the purpose of the experiment, the reactions were carried out in the presence or absence of L-[³⁵S]methionine (DuPont NEN) in the transcription-translation mixture. The in vitro translated products were then analyzed by electrophoresis in SDS-10% polyacrylamide gel, and used as the protein source in electrophoretic mobility shift assay (EMSA).

### (3) Electrophoretic Mobility Shift Assay

EMSA was conducted as previously described (Lee, H.-J. et al. (1995) J. Biol. Chem. 270, 30129-30133). The double-stranded NBRE oligonuleotides (5′-GATCGAGTTTTAAAAGGTCATGCTCAATTT-3′) and the −228TR4RB of the human 21-OHase gene were ³²P-end-labeled as probes. For cold competition reactions, unlabeled doubled-stranded oligonucleotides were mixed with the labeled probe prior to the addition to the reactions. For antibody supershift analysis, 1 μl of monoclonal anti-TR4 antibodies² (G232-416.3 and G232-85.6) was added into the reactions for 15 min at room temperature prior to loading on a 5% native gel (Lee, H.-J., and Chang, C. (1995) J. Biol. Chem. 270, 5434-5440; Lee, H.-J. et al. (1996) J. Biol. Chem. 271, 10405-10412). Radioactive bands were visualized by autoradiography with Fuji RX film, and quantified by PhosphorImager (Molecular Dynamics).

### (4) Competitive EMSA

To determine the relative binding affinity (RBA), EMSA was performed as above using the [³²P]-NBRE as a probe, except that increasing concentrations of unlabeled annealed competitors were mixed with the probe prior to adding into the reactions. Radiolabeled bands were quantified by PhosphorImager (Molecular Dynamics). The RBA was calculated from the relative ratio between the intensity of the specific band shift and that of the non-specific band shift. Competitive displacement curves were graphed as the competitor concentration (ng) versus the percentage of the intensity remaining in the specific band with 100% bound, representing the amount in the absence of competitor DNA as previously described (Yao, E. F., and Denison, M. C. (1992) Biochem. 31, 5060-5067; White, T. E. K., and Gasiewicz, T. A. (1993) Biochem. Biophys. Res. Commun. 193, 956-962). The IC₅₀ represents the competitor concentration that inhibits 50% of the RBA (Yao, E. F., and Denison, M. C. (1992) Biochem. 31, 5060-5067; White, T. E. K., and Gasiewicz, T. A. (1993) Biochem. Biophys. Res. Commun. 193, 956-962).

### (5) Scatchard Analysis

The DNA-protein binding affinity assay between the TR4 and either the NBRE or the −228TR4RE of the human 21-OHase gene was carried out as previously described (Lee, H.-J. et al. (1995) J. Biol. Chem. 270, 30129-30133) with modifications.

### (6) Cell Culture, Transfection Experiments, CAT, and Dual-Luciferase Reporter Gene Assays

The mouse Y-1 adrenocortical tumor cells, kindly provided by Dr. Bernard P. Schimmer, were cultured in Dulbecco′s modified Eagle's medium (Life Technologies) supplemented with penicillin (100 units/ml), streptomycin (100 μg/ml), amphotericin (0.25 μg/ml), and 15% heat-inactivated (56° C. for 30 min) fetal bovine serum (Harlan). Transient transfection of the Y-1 cells plated at an initial density of 3×10⁵/60-mm dish were transfected by the calcium phosphate precipitation method as previously described (Lee, H.-J. et al. (1995) J. Biol. Chem. 270, 30129-30133). A dual-luciferase reporter assay was performed according to the manufacturer's instructions (Promega). The pRL-TK plasmid (Promega) was co-transfected to normalize the transfection efficiency. Forty-eight hours after transfection, cells were washed twice with phosphate-buffered saline after removing the growth medium. Four-hundred μl of 1× passive lysis buffer were added to cover all the cells and the dish was rocked slowly several times at room temperature for 15 min according to the manufacturer's instructions (Promega). Cellular lysates were scraped, collected, and centrifuged in an Eppendorf centrifuge for 30's. Aliquots (20 μl) of the supernatants were assayed for both firefly and Renilla luciferase activities within the same reaction tube according to the manufacturer's instructions (Promega). Briefly, the supernatants were first mixed with 100 μl of luciferase assay buffer II containing beetle luciferin (Promega), and the light intensity of the reaction was measured in a luminometer Monolight 2010 (Analytical Luminescence Laboratory) using a modified mode (30 s). Second, 100 μl of Stop & Glo reagent containing coelenterazine were added and the light intensity was measured. Firefly luciferase activities were normalized with Renilla luciferase activities, and then averaged over at least four independent experiments with error bars designating standard deviations.

### (7) Statistics

Statistical comparisons of the control with the treated groups were performed by the Student's t test. The accepted level of significance was p<0.05.

b) Results

### (1) Characterization of the Binding Preference of the TR4 to Different DRs

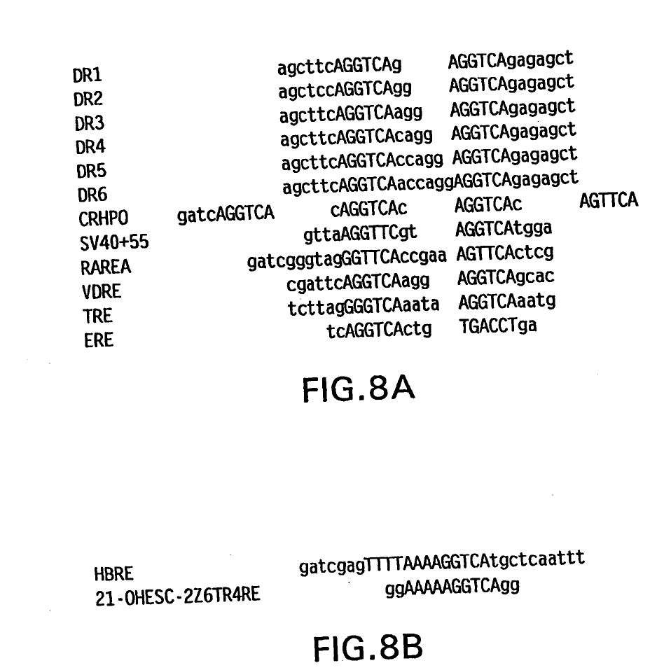

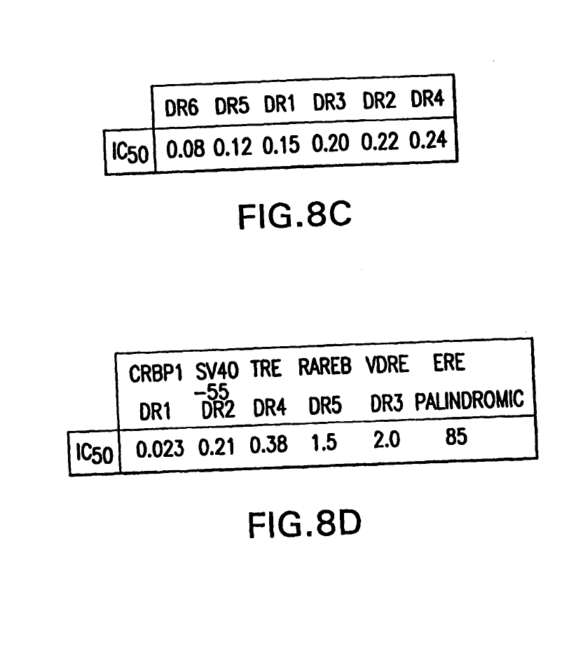

The data demonstrated that the TR4 can recognize and bind to tandem repeats of the AGGTCA motif in its target genes (Lin, D. L. et al. (1998) Endocrine 8, 123-134; Lee, H.-J. et al. (1995) J. Biol. Chem. 270, 30129-30133). Using cold competitive EMSA, the data suggested the binding preference of the TR4 to synthetic DRs (FIG. 8A) is in the order of DR6>DR5>DR1>DR3>DR2>DR4 with the IC₅₀ ranging slightly from 0.08 to 0.24 ng (FIG. 8B). For natural DRs, however, the TR4 has a dramatically different preference in the order of CRBPII (DR1)>SV40+55 (DR2)>TRE (DR4)>RAREβ (DR5)>VDRE (DR3)>ERE (palindrome) with the IC₅₀ varying widely from 0.023 to 85 ng (FIG. 8C). These data indicated that the TR4 has the ability to bind to various direct repeats of the AGGTCA consensus motif with different affinities.

### (2) Binding of the TR4 to a Monomeric Response Element

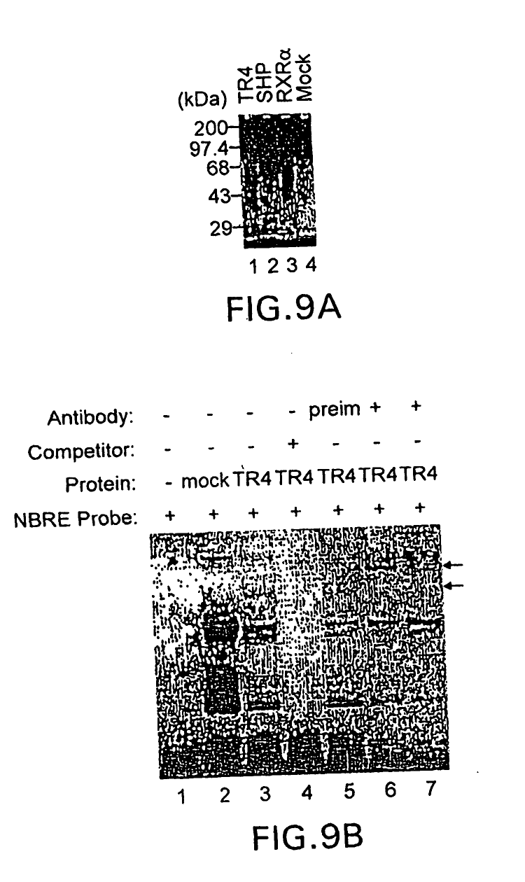

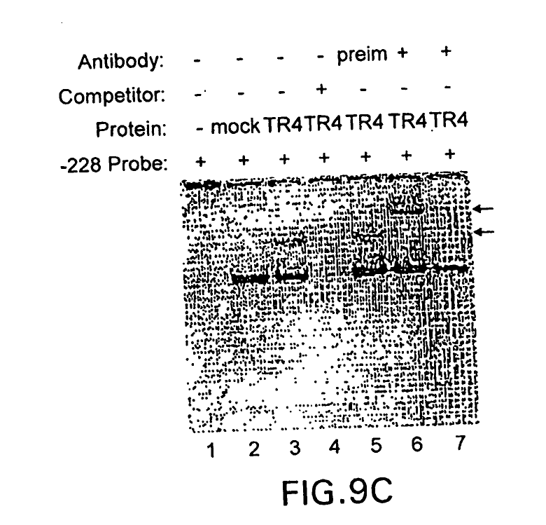

The TR4 protein was coupled transcribed and translated in vitro to produce a protein as shown in FIG. 9 for the source of EMSA study to show that the TR4 may also bind to a monomeric response element. Using a double-stranded NBRE oligonucleotide as a probe, a specific DNA-protein complex was visualized (FIG. 9A, lane 3, arrowhead). This radioactive DNA-protein complex was abolished in the presence of 50 fold molar excesses of unlabeled NBRE (lane 4). Moreover, the monoclonal anti-TR4 antibody (G232-416.3) could supershift this specific DNA-protein complex (lane 6, arrow). However, the monoclonal anti-TR4 antibody (G232-85.6) could eliminate such a complex (lane 7). These data indicated that the TR4 indeed has the ability to specifically bind to the monomeric NBRE.

To determine the DNA-protein binding affinity between the TR4 and a monomeric DNA response element in more detail, a Scatchard analysis was performed by EMSA (FIG. 9B). Constant amounts of the in vitro expressed TR4 were incubated with different concentrations of the NBRE (FIG. 9B). DNA-protein complexes were resolved in EMSA. Scatchard analysis revealed a single binding component for the specific DNA-protein complexes with dissociation constants (K_{d}) of 0.2 for the NBRE (FIG. 9B). These results indicated that the TR4 orphan receptor, like other orphan receptors, binds to a monomeric DNA response element with high affinity.

### (3) Repression of Gene Expression of the Human 21-OHase Gene by the TR4 Orphan Receptor

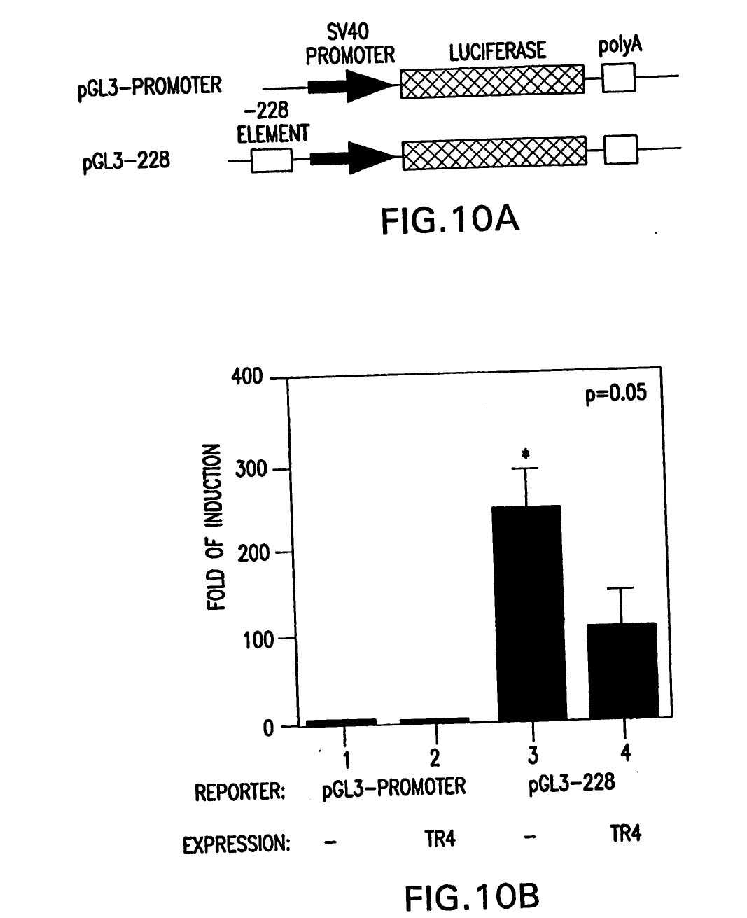

Analysis of the TR4 expression by both Northern blot analysis and in situ hybridization showed that the TR4 transcript was expressed in the adrenal gland (Chang, C. et al. (1994) Proc. Natl. Acad. Sci. USA. 91, 6040-6044; Young, W.-J. et al. (1997) J. Biol. Chem. 272, 3109-3116). To determine whether the TR4 plays a role in 21-OHase gene expression via interaction with the −228TR4RE, a dual-luciferase reporter assay in Y-1 adrenocortical tumor cells was performed. As shown in FIG. 10, Y-1 cells were co-transfected with a mammalian expression vector containing the full-length TR4 cDNA (CMX-TR4) and either the parent pGL3-promoter or the pGL3-228 reporter plasmid. The results showed that the TR4 can significantly suppress the luciferase reporter activity via the −228TR4RE of the 21-OHase gene.

Disclosed herein TR4 can bind to direct repeats of the AGGTCA core sequence in several response targets (Young, W.-J. et al. (1997) J. Biol. Chem. 272, 3109-3116; Lee, H.-J. et al. (1995) J. Biol. Chem. 270, 30129-30133) with high affinity. Thus, the TR4 could play roles in viral gene expression and appears to be involved in the cytokine pathway during neurogenesis. Recently, Hirose et al. showed that the TR4 forms a homodimer and binds to tandem repeats of the AGGTCA core motif (Hirose, T et al. (1995) Biochem. Biophys. Res. Commun. 211, 83-91). Other data also suggested that the TR4 may modulate gene expression mediated by retinoid (Lee, Y.-F. et al. (1998) J. Biol. Chem. 273, 13437-13443) and thyroid hormone signaling pathways (Lee, Y.-F. et al. (1997) J. Biol. Chem. 272, 12215-12220).

In the comparison of synthetic and natural HREs, the human TR4 showed a different binding preference toward these DNA response elements (FIG. 8). In the case of synthetic HREs, there was only a 2 fold difference in the binding affinity among DR1 to DP5. This indicates that the TR4 has a broad and non-discriminating ability to recognize synthetic DRs. In contrast, the binding affinity differed dramatically between the TR4 and natural response elements, in which CRBPII reached the highest with an 87 fold difference in binding affinity. In addition to direct repeats, the TR4 bound to palindromic ERE poorly, suggesting that direct repeats of the AGGTCA consensus motif function more effectively as HREs for the TR4 orphan receptor. This great variety in binding preference of the TR4 to different natural HREs strongly implies that not only the space between the direct repeats, but also the motif sequence, contributed to the specific binding of the TR4.

Detailed analysis of members of the steroid receptor superfamily have shown that dimerization produces novel complexes that bind DNA with unique properties, and thereby generate diversity in hormone response networks (Glass, C. K. (1994) Endocrine Rev. 15, 391-407). In contrast, little is known about the contributions of each monomer toward the transcriptional regulation of the complex. The data disclosed herein demonstrated that the monovalent HREs serve as binding targets for the TR4 orphan receptor. Such behavior would be in sharp contrast with classical family members, which may bind as either homo- or heterodimers with partners to two copies of the core recognition motif. The Nurr1-RXR heterodimer model raises the possibility that RXR associates with the monomeric HRE-bound Nurr1 in the absence of RXR-specific DNA contacts (Forman, B. M. et al. (1995) Cell 81, 541-550). This is consistent with the data disclosed herein that TR4 can form dimers in the DNA binding, while only one of them has specific DNA contacts. This is supported for example, by the fact that the migration position of the TR4-monovalent HRE complexes is the same as that of the TR4-bivalent HRE complexes (FIG. 9).

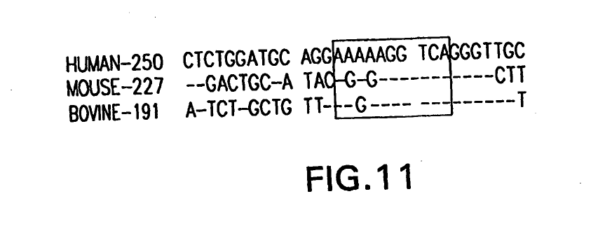

Comparison of the 5′ flanking sequences of the 21-OHase gene among human, mouse, and bovine revealed an important regulatory element which covers the major part of a highly conserved 40-bp region (Parker, K. L. et al. (1986) J. Biol. Chem. 261, 15353-15355). This regulatory region has been demonstrated to be required for expression of 21-OHase in Y-1 adrenal cells. Thus far, the nature of this regulatory element has not yet been fully determined. Deletion of this region most probably reflects changes in the decrease in transcriptional rates of 21-OHase transcripts (Parker, K. L. et al. (1986) J. Biol. Chem. 261, 15353-15355). As shown in FIG. 11, this regulatory region in the human gene is located between 253 and 203 nucleotides 5′ of the transcriptional initiation site. Interestingly, the AGGTCA core consensus sequence of a monomeric response element is highly conserved in the center of this regulatory region among all three species. In contrast, the perfect monomeric response element is only present in the human gene, while both mouse and bovine genes vary in the AT-rich preceding region (FIG. 11). As a result, the TR4 could have an additional regulatory effect on human 21-OHase gene expression via this monomeric −228TR4RE. The monomeric TR4 interactions can have a squelching or quenching effect on the transcription.

### 3. Example 3

### Modulation of the Retinoic Acid-Induced Cell Apoptosis and Differentiation by The Human TR4 Orphan Nuclear Receptor

Previously the TR4 orphan nuclear receptor (TR4) has been demonstrated to suppress RA-induced transactivation via a negative feedback control mechanism and in situ analysis showed that TR4 is extensively expressed in mouse brain, especially in regions where the cells are proliferating. A tetracycline-inducible system with antisense TR4 in teratocarcinoma P19 cell lines was generated to analyze the retinoic acid-induced differentiation of these cells. The results disclosed herein indicated that the expression of TR4 reduced by doxycycline-antisense TR4 would alter the retinoic acid-induced differentiation pathways that result in the changes of cell morphology and cell cycle profile. The data further indicated that the RA-induced apoptosis, judging by DNA fragmentation, could also be altered by the induction of antisense-TR4. Together, these findings provide in vivo evidence that an orphan nuclear receptor, such as TR4, can play major roles in the RA-mediated apoptosis or differentiation in P19 cells.

a) Materials and Methods

### (1) Plasmids

The plasmid used for the stable transfectants contains the anti-sense TR4 and was constructed as follows. The TR4 fragment containing the 5′-flanking region was released by EcoR I digestion from pBlueScript-TR4 (Chang, C. et al. (1994) Proc. Natl. Acad. Sci. USA 91, 6040-6044) and ligated to the ptetO₇-CMV-L plasmid, (Soriano, P. et al. (1991) Cell 64, 693-702) from which luciferase has been removed by Xba I digestion (PtetO₇-CMV-TR4). The orientation of TR4 was confirmed by Sac II digestion. A neo expression cassette, PGKneobpA (Tsai, M. et al. (1996) Biophys. J. 70, 2023-2029), which serves to select transfected cells, was inserted into ptetO₇-CMV-TR4 plasmid in an anti-sense orientation to the CMV promoter. The silencing-mediated protein (TetR-KRAB fusion protein) which was expressed by pCMV-tetR-KRAB-hyg plasmid was provided by Dr. H. Thiesen (Johnes-Villeneuve, E. M. V. et al. (1982) J. Cell. Biol. 94, 253-262).

### (2) Centrifugal Elutriation and Flow Cytometry

Briefly, 1-2×10⁸ P19 cells growing exponentially were centrifuged and collected into 20 ml of fresh αMEM medium, then loaded into the Sanderson separation chamber of a Beckman JE6 elutriator system (Beckman Instruments, Palo Alto, Calif.). The procedures of centrifugal elutriation and flow cytometry have been described previously (Tsai, M. et al. (1996) Biophys. J. 70, 2023-2029). Cells in each fraction were counted, and their volume distributions were measured immediately by a Coulter counter and channelyzer system (model c1000; Coulter Electronics, Hialeah, Fla.). About 0.5-1×10⁶ cells from each fraction were fixed for DNA analysis on an Epics Profile flow cytometer (Coulter Electronics, Hialeah Fla.). DNA histograms were analyzed according to the mathematical model of Fried et al. (Fried, J. et al. (1976) J. Cell Biol. 71, 172-181). The relationship between cell volume and cell cycle position was determined according to the method of Brosing et al. (prosing, J. W. et al. (1986) Radiat. Res. 105, 138-146).

### (3) Cell Culture and Establishing Anti-sense TR4 Expressing Cell Lines

The P19 mouse teratocarcinoma cells were maintained in αMEM containing 10% heat-inactivated bovine serum FBS). ptetO₇-CMV-αTR4-neo and pCMV-tetR-KRAB-hyg were co-transfected into p19 cells by using the lipofectin method and followed by double antibiotic, G418 at 500 μg/ml and hygromycin B at 300 μg/ml, selection for three weeks. The surviving cells were serially diluted in order to grow colonies from single cells.

### (4) Nuclear Extracts

Nuclear extracts were prepared following the mini-extract procedure (Lee, K. A. et al. (1988) Gene Anal. Tech. 5, 22-31). The cells, with or without treatment of doxycycline for at least 48 h, were harvested and lysed by pushing through a 25-gauge hypodermic needle. The nuclear pellet was resuspended in buffer C (500 mM NaCl, 20 mM HEPES, 25% glycerol, 1.5 mM MgCl₂, 0.2 mM EDTA, 0.5 mM DTT) and incubated on a rotating wheel for 30 min at 4° C. The nuclear debris was pelleted by centrifugation for 30 min and the supernatant was dialyzed for 2 h against buffer D (20 mM HEPES, 20% glycerol, 100 mM KCl, 0.2 mM EDTA, 0.5 mM DTT). Protein concentration was determined using Bradford reagent (Bio-Rad). Three μg of nuclear extract were used in each 20 μl DNA-protein interaction.

### (5) Northern Blotting Analysis

Anti-sense TR4 stable transfectant P19 cells (P19αTR4) were maintained in αMEM medium containing G418 (500 μg/ml) and hygromycin (300 μg/ml) either with or without 1 μg/ml doxycycline for at least 48 h before treatment. P19αTR4 cells were treated with 10⁻⁶ M all transRA (atRA) and harvested at 0, 24, 48 and 72 h after atRA treatment. Total RNA from the RA-treated P19αTR4 cells was prepared by the ultracentrifugation method as described previously (Chao, Y. C. et al. (1992) J. Virol. 66, 1442-1448). Probes were labeled with α-[³²P]dCTP by using a random primer DNA labeling system.

### (6) Electrophoretic Mobility Shift Assay (EMSA)

EMSA analysis was performed according to the methods described by Cooney et al. (Cooney, A. J. et al. (1993) J. Biol. Chem. 268, 4152-4160). The CNTFRα-DR1 oligonucleotides (Young, W.-J. et al. (1997) J. Biol. Chem. 272, 3109-3116) were 5′ end labeled with γ[³²P]ATP (specific activity=6000 Ci/mmole) (New England Nuclear) by T4 polynucleotide kinase to, 2-8×10⁸ cpm/μg. Labeled oligonucleotides were incubated with in vitro translated proteins. In order to confirm the band thought to be formed by the TR4-DR1 complex, 1 μl of anti-TR4 monoclonal antibody (#15) was added prior to the addition of labeled oligonucleotides. DNA-protein complexes were then resolved on a 5% non-denaturing polyacrylamide gel at 4° C. using 0.5×TBE buffer (1×TBE=0.09 M Tris-borate and 2 mM EDTA). Gels were fixed in 50% ethanol and 10% acetic acid for 30 min before drying. The radioactive gels were analyzed by either PhosphorImager (Molecular Dynamics, Inc) or autoradiography.

### (7) Western Blot Analysis

Western blot analysis was employed as previously described. Basically, nuclear extract was obtained from P19 parental or P19αTR4 stable transfectant cells after various treatments. Twenty μg of nuclear protein from each treatment was separated on 10% SDS-PAGE, transferred to an Immobilon-P membrane (Millipore), and detected with the anti-TR4 polyclonal antibody. Alkaline phosphatase-conjugated goat anti-mouse IgG (Santa Cruz Biotechnology) was used as the second antibody, and the color was then developed by utilizing the detection system (Bio-Rad).

### (8) DNA Fragmentation Assay

To examine DNA fragmentation, DNA isolation was performed according to the method described previously (Pringent, P. et al. (1993) J. Immunol. Methods 160, 139-140), with slight modification. Briefly, cells were harvested after treatment and washed twice with PBS. Then 1.2 ml of lysis buffer (10 mM Tris-HCl pH 8.0, 10 mM EDTA pH 8.0, 75 mM NaCl, 0.5% SDS and 0.15 mg/ml proteinase K) was added to 5×10⁶ cells and incubated at 50° C. for 3 h. The lysate was then spun down at 14,000×g for 20 min at room temperature. The gelatinous pellet was removed with a pipette and the supernatant was digested with 100 μg/ml RNase A at 37° C. for 30 min, followed by digestion with 100 μg/ml proteinase K at 50° C. for 30 min. The DNA was precipitated by adding an equal volume of 100% ethanol and NaCl such that the final concentration was 0.5M NaOH. Following centrifugation, the pellet was washed with 70% ethanol and resuspended in 50 μd of TE buffer. DNA ladders were visualized by running on a 1.5% agarose gel.

b) Results

### (1) Higher Expression Level of TR4 in S-phase in Untreated P19 Cells

Exponentially growing P19 cells were separated into fractions based on their cell size difference. Shown in FIG. 12A (a-f) is the cell cycle distribution of five populations of P19 cells (unseparated, G1, G1/S, S, early G2 enriched, G2/M) separated by centrifugal elutriation. DNA histograms for these cell populations were analyzed by flow cytometry. The results indicated that cell populations enriched in GI (>86.4%), S (>84.8%) and G2M (85.8%) cells can be obtained by centrifugal elutriation. Each fraction was collected for Northern analysis. As shown in FIG. 12B, the expression of TR4 mRNA is 2-3 fold higher in S-phase than those in G1 and G2/M phases (FIG. 12B, lane 3 vs lanes 1 and 5), suggesting that TR4 is not expressed equally during cell cycle progression. β-actin expression was used here as a loading control. These results are in agreement with the in situ analysis of TR4 expression during the development of embryos (Young, W.-I. et al. (1998) J. Biol. Chem. 273, 20877-20885), which indicated that TR4 might play some roles during of cell cycle regulation.

### (2) The Strategy for Establishing Stable Transfectants Expressing Anti-sense TR4

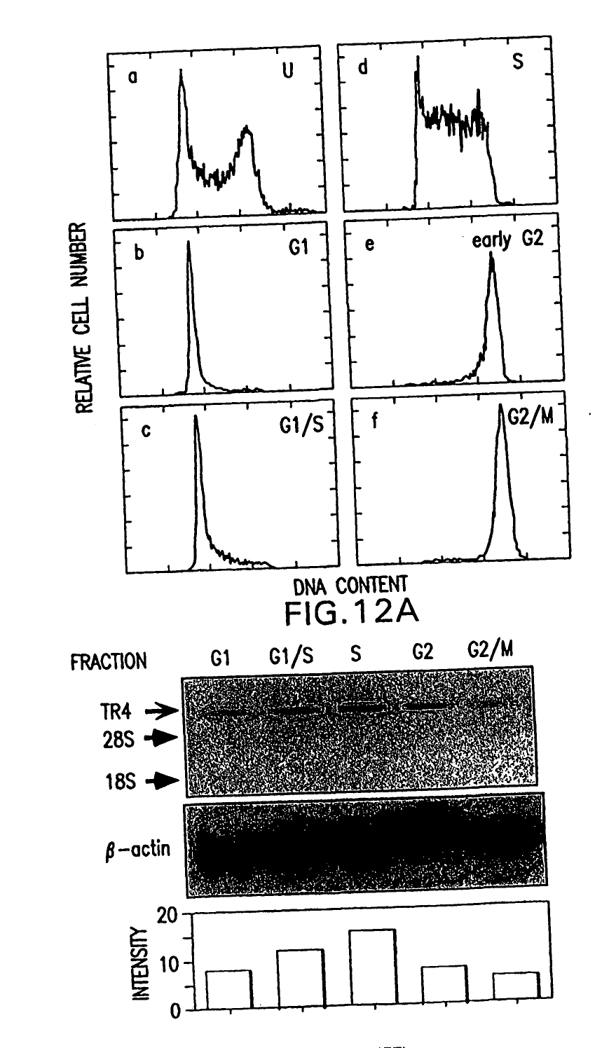

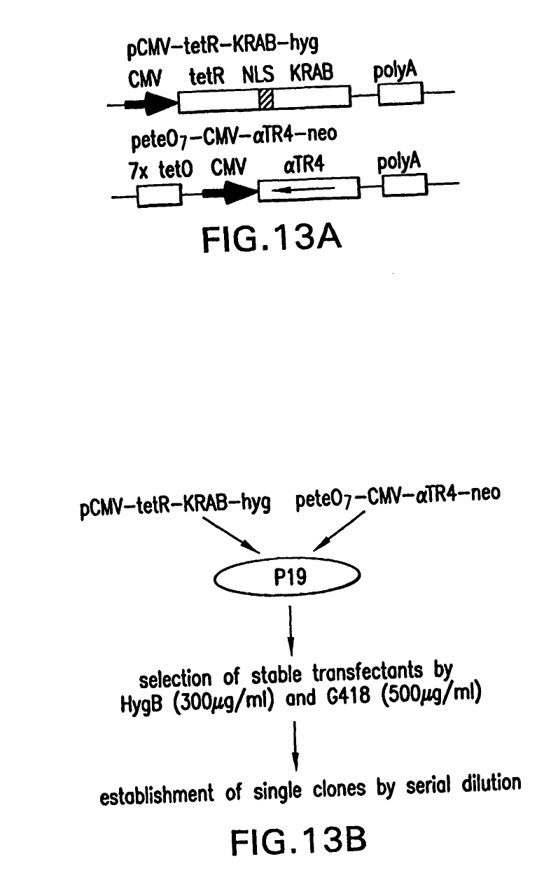

To further study the potential roles of TR4 in cell cycle-related apoptosis or differentiation, a tetracycline-inducible system was applied in P19 cells to monitor the expression of TR4. As shown in FIG. 13, in order to control the expression of TR4, two expression plasmids, pCMV-tetR-KRAB-hyg and ptetO7-CMV-αTR4-neo, were co-transfected into P19 cells using the lipofectin method. In the expression plasmid pCMV-tetR-KRAB-hyg (Johnes-Villeneuve, E. M. V. et al. (1982) J. Cell. Biol. 94, 253-262), the tetracycline-responsive transcriptional silencer protein, TetR-KRAB, was placed downstream of the strong immediate-early human CMV promoter, followed by an SV40 polyadenylation signal (poly A). A nuclear localization sequence (NLS) was inserted to favor nuclear localization of the TetR-KRAB fusion protein in mammalian cells. The target plasmid, ptetO₇-CMV-αTR4-neo, was driven by a CMV promoter to express anti-sense TR4. In this construct, seven tetO sequences that can be bound by TetR-KRAB were placed upstream of CMV promoter. Binding of the KRAB domain to the tetO sites results in active repression of anti-sense TR4 expression. Addition of tetracycline prevents TetR-KRAB from binding to the tetO sequence and thereby restores the transcriptional activity. To establish stable transfectants, the selective antibiotics neo and hygromycin B were also inserted into these two expression vectors, respectively. After co-transfection of these two plasmids, the cells were selected by growing with G418 (500 /μg/ml) and hygromycin B (300 μg/ml) for two to three months. Resistant colonies were serially diluted to obtain a single clone. After selection and amplification, 7 clones were obtained, each representing an individual population.

The use of this type of system first, allowed the avoidance of potential artifacts associated with chronic high levels of anti-sense TR4 expression. Secondly, it allowed the comparison of the behavior of the same genetically homologous clones under inducing and non-inducing conditions, therefore factoring out clonal variations. By using a tetracycline-inducible system with the stable transfectant, under the same genetic conditions, we can directly compare the effects from the blockage of TR4 expression with those of unblocked expression. Such a method, of course, opens up many exciting perspectives for the study of TR4 function in vivo.

### (3) Characterization of the Stable Transfectants

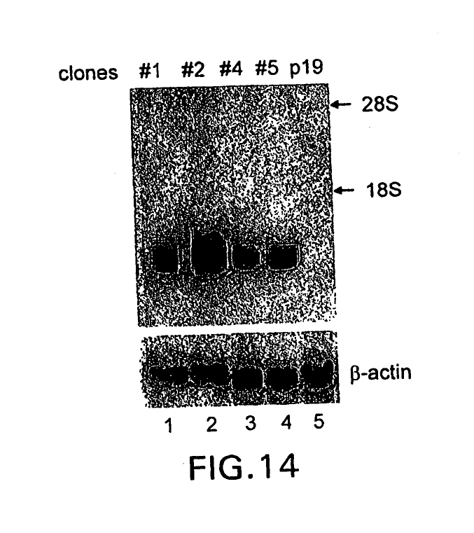

Northern blotting analysis was performed to confirm that the selected cells contain the transfected plasmids. The probe used was [³²P]-dCTP-neo because there are no endogenous neo transcripts in P19 cells and only the cells that contain the exogenously transfected plasmids can be detected. As shown in FIG. 14, a band was detected in all the clones we tested (FIG. 14, lanes 1 to 4). In contrast, no band was detected in the parental P19 cells (FIG. 14, lane 5). A similar result was obtained when a [³²P]-hygromycin probe was hybridized (data not shown). P-actin was used as an internal control to normalize an equal loading amount of total RNA. These data demonstrate that the clones selected did contain the plasmids transfected.

### (4) Doxycycline Tightly Controls the Expression of TR4 in the 19αTR4 Stable Transfectant

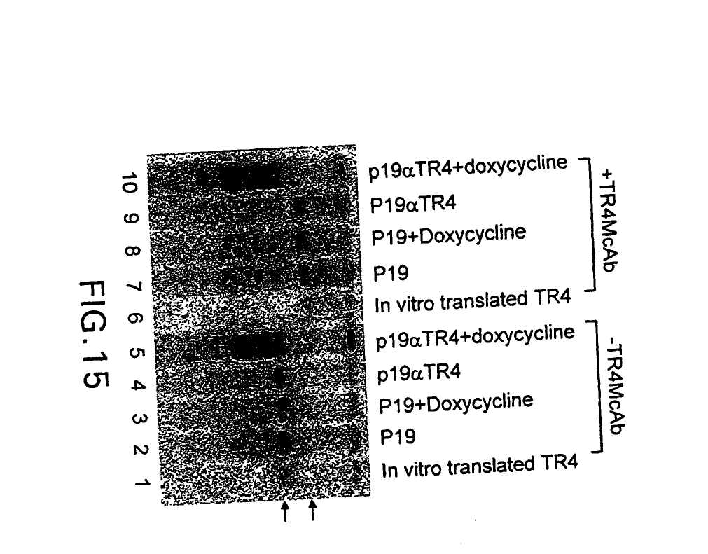

After it was confirmed that these stable transfectants express neo and hygromycin B transcripts, the tetracycline-inducible system was assessed in these stable transfectants. EMSA with [³²P]-CNTFR-DR1 DNA oligomer probe was used to determine the functional TR4 protein in P19 nuclear extracts in both induced and non-induced systems. As shown in FIG. 15, parental P19 cells showed the same binding patterns with or without doxycycline treatment (FIG. 15, lanes 2, and 3). One population of proteins shifted to the same position as in vitro translated TR4 protein (FIG. 15, lane 1). In contrast, the binding patterns with or without doxycycline treatment were different in the stable transfectant P19αTR4 (FIG. 15, lanes 4 and 5). In order to confirm which binding population contained TR4, a specific anti-TR4 monoclonal antibody was used. A supershifted band formed by DR1-TR4-antibody was observed with or without doxycycline-treatment (FIG. 15, lanes 7 and 8). The same supershifted band was observed in parental P19 cells and the P19αTR4 cells without the doxycycline (FIG. 15, lane 9). This DR1-TR4-antibody complex had the same migration position as in vitro translated TR4 (FIG. 15, lane 6). In contrast, no visible band could be detected when we added the anti-TR4 antibody to the doxycycline-treated P19αTR4 nuclear extract (FIG. 15, lane 10 vs 9). These data clearly demonstrate that the expression level of functional TR4 could be blocked by anti-sense TR4, which is tightly controlled by doxycycline.

### (5) The Morphological Change of P19αTR4 During RA-treatment

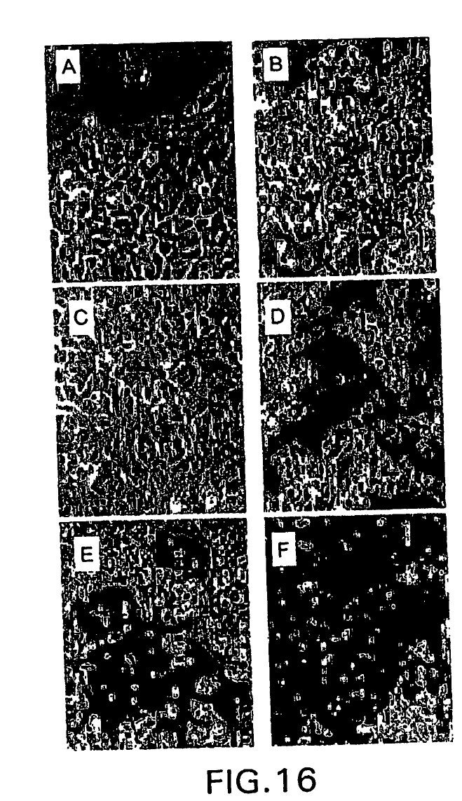

During normal development of the nervous system, large numbers of neurons degenerate and die naturally by a process of programmed cell death, known as apoptosis. Apoptosis eliminates 20-80% of neurons during development (Hamburger, V., and Oppenheim, R. W. (1982) Neurosci. Commun. 1, 39-55; Oppenheim, R. W. (1991) Annu. Rev. Neurosci. 14, 453-501). In order to understand the role of TR4 in RA-induced cell differentiation and apoptosis, we treated the P19αTR4 cells with 10⁻⁶ M of RA for five days either with or without treatment of doxycycline. The parental P19 cells were tested as a control. All the cells were collected and stained with 0.04% trypan blue to measure viability. As shown in FIG. 16, in the absence of doxycycline, 25-30% of P19αTR4 cells started to round up, detach, and die after 24 h of RA treatment (FIG. 16B). Forty-eight h after RA-treatment the cells exhibited the same phenomena with even more extensive cell death (FIG. 16D), in that about 40% of the cells detached from the cell culture plates and died. Seventy-two h after RA-treatment, around 60-70% of the cells rounded up, detached, and finally died (FIG. 16F). On the other hand, inhibition of TR4 expression by doxycycline-induced antisense TR4 delayed this process as judged from both the morphology and cell viability tests in P19αTR4 cells (FIG. 16A, C, and E). In contrast, the cell death induced by RA-treatment was not observed until after 72 h (FIG. 16E) in cells without antisense TR4 expression.

### (6) Inhibition of TR4 Expression Delays RA-induced Cell Apoptosis

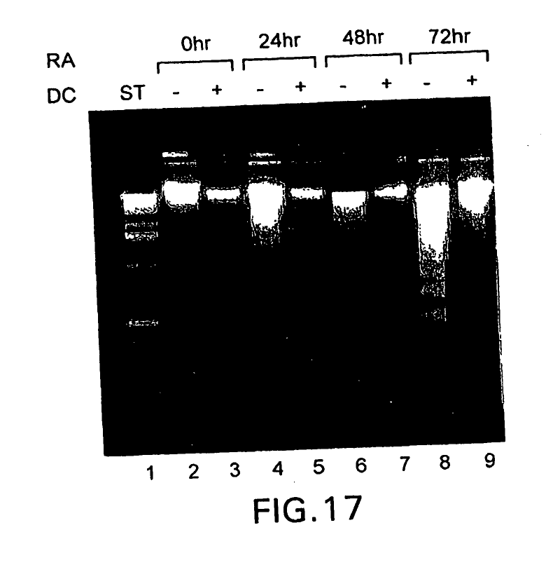

From the cell morphology and viability tests disclosed herein, it was concluded that blockage of TR4 expression could slow the cell death process. A DNA fragmentation assay was applied to confirm this process follows the pathway of apoptosis. As shown in FIG. 17, the DNA ladder was first observed in the P19αTR4 cells treated with RA for 24 h (FIG. 17, lane 4) without adding doxycycline. Formation of the DNA fragmentation in P19αTR4 cells became more and more obvious when the cells were continually treated with RA for 48 to 72 h in the absence of doxycycline (FIG. 17, lanes 6 and 8). In contrast, in the presence of doxycycline, the DNA ladder was not detected until 72 h of RA-treatment (FIG. 17, lane 9 vs. lanes 3, 5, and 7). These results strongly support that blockage of TR4 expression by the presence of doxycycline delays the process of apoptosis induced by RA-treatment as judged by both cell morphology and DNA ladder formation. Together, these data indicate that TR4 may play important roles in the modulation of RA-induced apoptosis and cell differentiation.

Several in vitro studies demonstrate that TR4 acts as a modulator in various steroid/thyroid hormone pathways (Lee, Y.-F. et al. (1997) J. Biol. Chem. 272, 12215-12220; Young, W.-J. et al. (1997) J. Biol. Chem. 272, 3109-3116; Lee, H.-J. et al. (1995) J. Biol. Chem. 270, 30129-30133; Young, W.-J. et al. (1998) J. Biol. Chem. 273, 20877-20885). In situ hybridization data also showed that TR4 could be expressed with spatiotemporal patterns in the mouse embryo, especially in the regions where the cells are undergoing proliferation. This inducible system has certain advantages

Apoptosis is an important concept because it focuses attention on the natural turnover of cells necessary for proper maintenance of a healthy organism. The occurrence of cell death in the developing nervous system may serve a variety of functions, including morphogenesis, regulation of target innervation, removal of cells that make projection or synaptic errors, removal of transient synaptic targets, and the removal of cells that provide transient guidance cues for axonal pathway formation. It is probable that multiple biochemical pathways result in morphological apoptosis. Disclosed herein it was found that blockage of TR4 expression by doxycycline induction of anti-sense TR4 delays the apoptosis response induced by RA treatment, consistent with TR4 being a regulator involved in the process of apoptosis. Following the RA treatment of P19 cells, TR4 expression was up-regulated at both the mRNA and functional protein levels (Lee, Y.-F. et al. (1998) J. Biol. Chem. 273, 13437-13443). These data are consistent with TR4 participation in building up and regulating a hierarchical network of transcription factors that establish normal development of the P19 cell. TR4 can function as a positive regulator that triggers the cascade of specific biosynthetic events needed for cell death. TR4's role in apoptosis can be parallel to other pathways involved in apoptosis. Whether other pathways converge on a final, late and lethal path remains to be determined. Several genes have been reported to regulate RA-induced apoptosis during P19 embryonic carcinoma cell differentiation. For example, wild type p53 can act to induce apoptosis (Wu, X., and Levine, A. J. (1994) Proc. Natl. Sci. USA 91, 3802-3806; Strasser, A. et al. (1994) Cell 78, 329-339; Attardi, L. D. et al. (1996) EMBO J. 15, 3693-3701); Rb may play a role in ensuring growth arrest, and abrogation of Rb function leads to apoptosis (Richon, V. et al. (1992) Cell Growth Differ. 3, 413-420; Gu, W. et al. (1993) Cell 72, 309-324; Schneider, J. W. et al. (1994) Science 264, 1467-1471); Bcl-2 can inhibit RA-induced apoptosis during the neural differentiation of embryonic carcinoma cells (Okazawa, H. et al., (1996) J. Cell Biol. 132, 955-968). The data disclosed herein indicated that the TR2 orphan nuclear receptor sharing similar functions with TR4 receptor (Lee, H.-J. et al. (1995) J. Biol. Chem. 270, 30129-30133; Lin, T.-M. et al. (1995) J. Biol. Chem. 270, 30121-30128; Lee, H.-J. et al. (1996) J. Biol. Chem. 271, 10405-10412; Lin, D.-L., and Chang, C. (1996) J. Biol. Chem. 271, 14649-14652), could also mediate the expression of p53 and Bc12, further strengthening the roles of TR2 and TR4 in apoptosis.

Retinoic acid affects several processes in cell proliferation and differentiation (Mendelsohn, C. et al. (1992) Dev. Biol. 152, 50-61). The effect of RA on gene expression is mediated via retinoic acid receptors (RARs) and retinoid X receptors (RXRs) (Petkovich, M. et al. (1987) Nature 330, 444-450; Giguere, V. et al. (1987) Nature 330, 624-629). Development of multicellular organisms is thought to be controlled by sequential activation and repression of a complex cascade of regulatory genes. TR4 represses the transcriptional activities of RA target genes by competing with RAR/RXR for binding to the same HREs. Not only TR4, but also other orphan nuclear receptors such as COUP-TFs, could repress the RA-mediated gene expression (Mangelsdorf, D. J. et al. (1992) Genes Dev. 6, 329-344; Cooney, A. J. et al. (1993) J. Biol. Chem. 268, 4152-4160). In contrast, there are several genes that are activated in P19/RA cells through the RA-response elements, such as jun, fos (de-Groot, R. P. et al. (1990) Nucleic Acids Res. 18, 3195-3202), and Egr-1 (Edwards, S. A. et al. (1991) Dev. Biol. 148, 165-173). These genes are activated in the presence of these negatively regulating orphan receptors, which can compete with the RXR/RAR heterodimers. It is likely that the net effect of the target genes on transcription depends on the concentration and affinity of the different receptors for their recognition sites, and thus the full context of the promoter-enhancer elements must be taken into account. RA could also increase the binding of nuclear proteins to the RA responsive elements, which includes TR4 and COUP-TFs. The dynamic balance between these negative regulators (TR4, COUP-TFs), positive regulators (jun, fos, and Egr-1) and RAR/RXR heterodimers in the specific target tissues, can influence the activity of target genes.

TR4 plays a central role in RA-mediated gene regulation and in the control of apoptosis and differentiation in EC cells.

### 4. Example 4

### Differential and Bidirectional Regulation Between TR2 and TR4

### Orphan Nuclear Receptors and a Specific Ligand Mediated-Peroxisome Proliferator-Activated Receptor α (PPARα) α in Human HaCaT Keratinocytes

TR2 orphan nuclear receptor (TR2) can modulate the transcriptional activity of the reporter gene containing an AGGTCA direct repeat-hormone response element. The role and regulation of TR2 in human HaCaT keratinocytes is disclosed. Using a chloramphenicol acetyltransferase reporter gene assay, the data demonstrated that TR2 could suppress retinoic acid (RA)-induced transactivation by 44.7% in human HaCaT keratinocytes. This suppression is similar to 47.3% suppression of RA-induced transactivation by TR4 orphan nuclear receptor (TR4), a steroid/ thyroid hormone receptor superfamily member very closely related to TR2. In contrast to this similar suppression of RA-mediated transactivation by TR2 and TR4, the data demonstrated that TR4, but not TR2, can significantly repress (95%) the Wy-14643-mediated peroxisome proliferator-activated receptor α (PPARα) transactivation. Western blot analysis further suggested that Wy-14643 can differentially regulate the expression of TR2 and TR4 (by increasing the expression of TR4 protein and decreasing that of TR2) in human HaCaT keratinocytes. These data demonstrate that close members of orphan nuclear receptors group, such as TR2 and TR4, can have distinct functions, but it also indicates the existence of differential bi-directional regulation between PPARα and TR2/4.

a) Materials and Methods

### (1) Cell Culture

Human HaCaT keratinocytes (originated in Dr. Norbert Fusenig's laboratory), an immortalized cell line, were cultured in Dulbecco's modified Eagle's medium plus 10% fetal calf serum and 100 U/ ml penicillin/ streptomycin. For Wy-14643 and vitamin D treatment, the medium containing charcoal-stripped fetal calf serum was used. Neonatal foreskins were obtained with informed consent according to the regulations of the University of Rochester Human Subjects Review Board. Keratinocyte cultures were established as described previously (Boukamp, P. et al. (1988) J Cell Biol 106, 761-771), and cultured in keratinocyte growth medium containing 30 μg/ml bovine pituitary extract, 0.1 ng/ml human epidermal growth factor, 5.0 μg/ml insulin, and 0.5 μg/ml hydrocortisone (KGM; Clonetics). For the experiment, the third passage of foreskin keratinocytes was used. When keratinocyte differentiation was induced by high Ca²⁺ switch, the keratinocyte growth medium without bovine pituitary extract, human epidermal growth factor, insulin, and hydrocortisone was used.

### (2) Plasmid

To construct the pCMV-TR2 plasmid, the full-length TR2 cDNA was ligated into the Sma I site of pCMV. The pCMX-TR4 plasmid was constructed as described previously (Lee, H.-J. et al. (1995) J. Biol. Chem. 270, 30129-30133). To construct pRARβ-CAT, the double-stranded oligonucleotides of retinoic acid response element (GGTTCAccgaaAGTTCA) from the promoter region of retinoic acid receptor β (RARβ) were synthesized (Haake, A. R., and Cooklis, M. (1997) Exp Cell Res 231, 83-95) and ligated it into the Bgl II site of pCATpromoter vector (Promega). The reporter plasmid P450cc24-CAT, which contains 5′-flanking region (−2200 to +188) of the rat vitamin D₃ 24-hydroxylase gene, was kindly provided by Dr. Y. Kato (Sucov, H. M. et al. (1990) Proc Natl Acad Sci USA 87, 5392-5396). PPRE₃-tk-LUC, the luciferase reporter plasmid containing the peroxisome proliferator response element (PPRE), obtained from the rat acyl-CoA oxidase promoter, was a kind gift from Dr. R. M. Evans (Ohyama, Y. et al. (1994) J Biol Chem 269, 10545-10550).

### (3) Coupled In Vitro Transcription and Translation

The full-length TR2 and TR4 were in vitro transcribed and translated by the rabbit reticulocyte-based transcription/translation kit (TNT coupled reticulocyte lysate system) (Promega) according to the manufacturer's instructions.

### (4) Transient Transfection

Polybrene with DMSO shock was used for transient transfection into HaCaT keratinocytes as reported previously (Kliewer, S. A. et al. (1992) Science 358, 771-774). Four μg of CAT constructs were co-transfected with 0.01-2 μg of pCMV-TR2 or pCMX-TR4. To normalize the transfection efficiency, 1 μg of β-galactosidase expression vector, pCMβ (Clontech), was co-transfected in all experiments.

### (5) CAT Assay

Twenty-four hours after transfection, cells were treated with 10⁻⁶ M all-trans retinoic acid, 10⁻⁷ M 1,25-dihydroxyvitamin D₃ (1,25—(OH)₂D₃), or ethanol as a vehicle. Forty-eight hours after transfection, the cells were washed twice with phosphate-buffered saline (−) and harvested. The cells were lysed by three freeze-thaw cycles. The lysates were centrifuged for 5 min at 14000 rpm. To inactivate the internal inhibitory fraction for CAT activity, the supernatants were incubated at 57° C. for 10 min and used in CAT reaction. The reaction products were dissolved with ethyl acetate and applied to thin-layer chromatography using a solvent of 95% chloroform and 5% methanol. CAT activity was measured by PhosphorImager (Molecular Dynamics).

### (6) Luciferase Assay

Twenty-four hours after transfection, 10⁻⁵ M Wy-14643, a ligand specific for PPARα, was added into the culture. Forty-eight hours after transfection, the cells were harvested and the luciferase activity was assayed according to the manufacturer's instructions (Promega).

### (7) Electrophoretic Mobility Shift Assay

Electrophoretic mobility shift assay analysis was performed as previously described (Jiang, C. K. et al. (1991) J Invest Dermatol 97, 969-973). The oligonucleotides were 5′ end labeled with γ-³²P ATP (specific activity=6000 Ci/mmole) (New England Nuclear) by T4 polynucleotide kinase. The specific activity reached 2-8×10⁸ cpm/μg. Labeled oligonucleotides (0.1 ng) were incubated with in vitro translated proteins, in the presence or absence of unlabeled oligonucleotide competitor or rabbit antiserum against TR2 (Cooney, A. J. et al. (1993) J Biol Chem 268, 4152-4160), for 15 min at room temperature. The binding buffer contained 25 mM Tris-HCl (pH 8.0), 10% glycerol, 50 mM KCl, 2 mM EDTA, 0.05% TritonX-100, 5 mM DTT, 1% β-mercaptoethanol, 5 ng/ml bovine serum albumin, 1.5 units/ml aprotinin, and 1 μg poly(dI.dC) (Pharmacia). DNA-protein complexes were resolved on a 5% non-denaturing polyacrylamide gel at 4° C. in 0.5×TBE buffer (1×TBE=0.09 M Tris-borate and 0.002M EDTA). Gels were fixed in 50% ethanol and 10% acetic acid for 30 min, followed by drying. The radioactive gels were analyzed by PhosphorImager (Molecular Dynamics). The double-strand oligonucleotides of PPRE (GTCGACAGGGGACCAGGACAaAGGTCACGTFCGGGAGTCGAC), obtained from the rat acyl-CoA oxidase promoter (Ohyama, Y. et al. (1994) J Biol Chem 269, 10545-10550), were used as a probe.

### (8) Western Blot Analysis

Before treatment with Wy-14643 or 1,25-(OH)₂D₃, the cells were maintained for one week in Dulbecco's modified Eagle's medium containing 10% charcoal-stripped fetal calf serum, and then either 10⁻⁵ M WY-14643 or 10⁻⁷ M 1,25-(OH)₂D₃ was added to HaCaT keratinocytes cultured on 100-mm dishes at subconfluency. At the indicated time after the treatment, keratinocytes were washed twice with ice-cold phosphate-buffered saline (−) and extracted with the extraction buffer (1% NP40, 1 μg/ml aprotinin, and 400 mM NaCl in 20 mM HEPES, pH 7.2). Protein concentration was determined using Bradford reagent (Bio-Rad). Twenty or forty μg per lane of nuclear extract and cell lysate protein from keratinocytes were loaded onto 10% polyacrylamide gel, and transferred to an Immobilon-P membrane (Millipore). The membrane was soaked overnight in 5% skim milk in phosphate-buffered saline (−)/0.05% Tween-20 at 4° C. and then incubated with anti-TR2 monoclonal IgM (8, 13) at a 1:800 dilution in 5% skim milk in phosphate-buffered saline (−)/0.05% Tween-20 for 2 hours at room temperature. After being washed three times at intervals of 10 min with phosphate-buffered saline (−)/0.05% Tween-20, the membranes were incubated with alkaline phosphatase-conjugated goat anti-mouse IgM (PharMingen) at a 1:1000 dilution for one hour at room temperature. Detection was performed with Alkaline Phospotase Conjugate Substrate Kit (Bio-Rad). For analysis of the expression level of TR2 protein during differentiation, normal human keratinocytes were first cultured in KGM with 0.15 mM Ca²⁺. When the keratinocyte cultures reached subconfluency, the Ca²⁺ concentration was increased to 1.8 mM. The cells were harvested at 0, 24, 48, and 72 hours after addition of Ca²⁺. The cells were pelleted by centrifugation, and lysed in 2% sodium dodecyl sulfate. The lysate was sonicated and the protein concentration was measured (23). Twenty μg of protein was subjected to Western blot analysis as described above. The expression amounts of TR2 were quantified by collage image analysis software (Fotodyne).

b) Results

### (1) TR2 Protein Is Expressed in HaCaT Keratinocytes

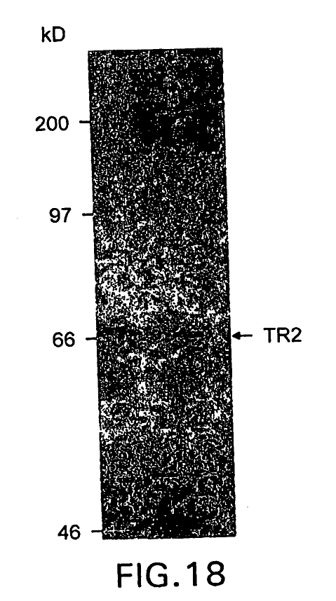

To examine whether TR2 is expressed in human HaCaT keratinocytes, Western blot analysis of their cell lysates was performed. The TR2 protein with a molecular weight of 67 kDa was detected with monoclonal anti- TR2 antibody (FIG. 18). With the isotype control IgM, this band was not detected (data not shown). The specificity of this monoclonal antibody has been confirmed previously (Young, W.-J. et al. (1998) J. Biol. Chem. 273, 20877-20885; Lee, H.-J. et al. (1996) J. Biol. Chem. 271, 10405-10412).

### (2) TR2 Protein Level Is Increased During Normal Human Keratinocyte Differentiation

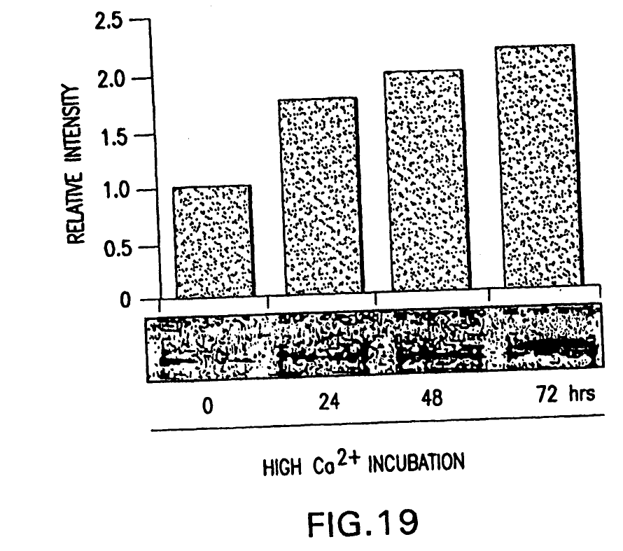

The expression level of TR2 protein during keratinocyte differentiation was tested. For this purpose, normal human keratinocytes are preferred because their differentiation can be easily induced by Ca²⁺ switch (Gibson, D. F. C. et al. (1996) J Invest Dermatol 106, 154-161). At first, normal human keratinocytes were maintained in KGM with 0.15 mM Ca²⁺. When the keratinocyte cultures reached subconfluency, the Ca²⁺ concentration was switched to 1.8 mM to induce differentiation. The cells were then harvested at 0, 24, 48, and 72 hours after addition of Ca²⁺, followed by cell lysis and Western analysis. At 24, 48, and 72 hours, TR2 protein expression increased to 1.72-, 1.92- and, 2.08-fold, respectively (FIG. 19), indicating that TR2 is up-regulated during differentiation of normal human keratinocytes. This increase of TR2 during differentiation is similar to that of TR4 in our previous report.

### (3) TR2 Suppresses RA-induced Transactivation in HaCaT Keratinocytes

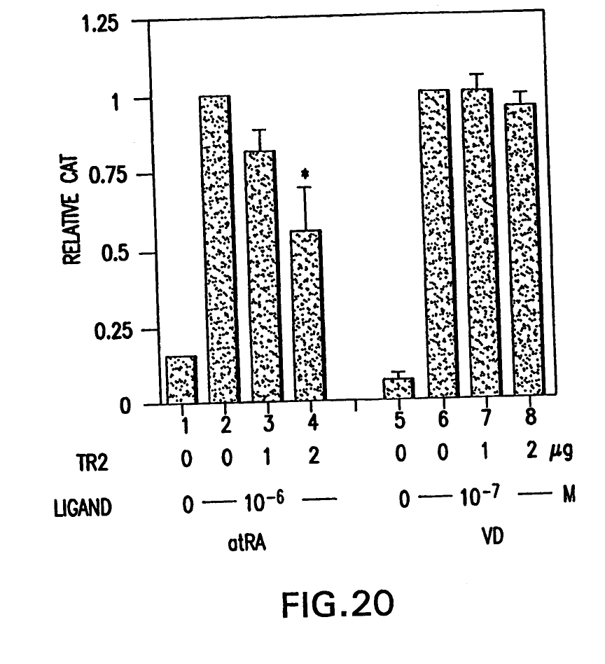

TR2 suppresses RA-induced transactivation in CV1 cells (Lin, T.-M. et al. (1995) J. Biol. Chem. 270, 30121-30128). Furthermore, TR4 can suppress RA-, but not vitamin D-induced transactivation in human HaCaT keratinocytes. Here the potential effect of increased TR2 expression on RA- and vitamin D-induced transactivation in HaCaT keratinocytes by a CAT reporter gene assay is disclosed. To assay the RA- and vitamin D-induced transactivation, the pRARβ-CAT reporter plasmid that contains the synthetic retinoic acid response element (GGTTCAccgaaAGTTCA) sequence obtained from the promoter region of RARβ (Haake, A. R., and Cooklis, M. (1997) Exp Cell Res 231, 83-95), and P450cc24-CAT that contains 5′-flanking region (−2200 to +188) of the rat vitamin D₃ 24-hydroxylase gene (Sucov, H. M. et al. (1990) Proc Natl Acad Sci USA 87, 5392-5396) were used. To analyze ligand-induced transactivation of CAT reporter plasmid via endogenous cognate receptors in HaCaT keratinocytes, exogenous RARs and vitamin D receptor were not transfected. We observed a 6.3-fold induction by 10⁻⁶ M all-trans RA in the absence of TR2 expression vector (FIG. 20, lane 1 vs. 2). Co-transfection of 1 and 2 μg of pCMV-TR2 decreased this CAT activity by 18.3% and 44.7%, respectively (FIG. 20, lane 3 and 4). Although we detected a 15.9-fold induction by 10⁻⁷ M of 1,25-(OH)₂D₃ without exogenous TR2, co-transfection of 2 μg of pCMV-TR2 did not have a significant effect on CAT activity (FIG. 20, lane 7-8). Co-transfection of pCMV-TR2 had no effect on the basal (ligand-independent) CAT activity. These results indicated that TR2 could suppress RA-, but not vitamin D-induced transactivation in human HaCaT keratinocytes.

### (4) TR2 Can Bind Specifically to PPRE

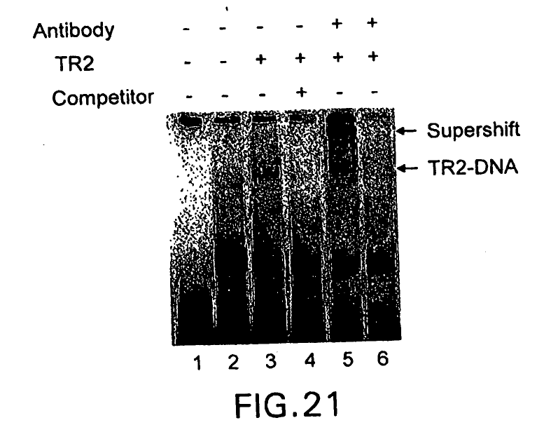

Recently, it has been reported that PPARα ligands can enhance keratinocyte differentiation (Hanley, K. et al. (1998) J Invest Dermatol 110, 368-375) and data disclosed herein (FIG. 19) demonstrated that TR2 is increased during keratinocyte differentiation. Therefore, it will be of interest to examine whether there is any cross-talk between these two receptor signaling pathways. First, TR2 can bind to PPRE using an electrophoretic mobility shift assay. The in vitro translated Th2 was incubated with ³²P-labeled PPRE (GTCGACAGGGGACCAGGACAaAGGTCACGTTCGGGAGTCGAC), obtained from the rat acyl-CoA oxidase promoter (Ohyama, Y. et al. (1994) J Biol Chem 269, 10545-10550). A specific TR2-PPRE complex was shown in the presence of TR2 (FIG. 21, lane 3), but absent in the mock translated control (FIG. 21, lane 2). This TR2-PPRE complex could be abolished with a 100-fold molar excess of the unlabeled PPRE (FIG. 21, lane 4). Moreover, this retarded complex was supershifted in the presence of the rabbit anti-TR2 serum (FIG. 21, lane 5), but could not be seen with the preimmune rabbit serum (FIG. 21, lane 6). Together, these data clearly demonstrated that TR2 could bind specifically to PPRE.

### (5) TR2 and TR4 Differentially Suppress PPAR Transactivation in HaCaT Keratinocytes

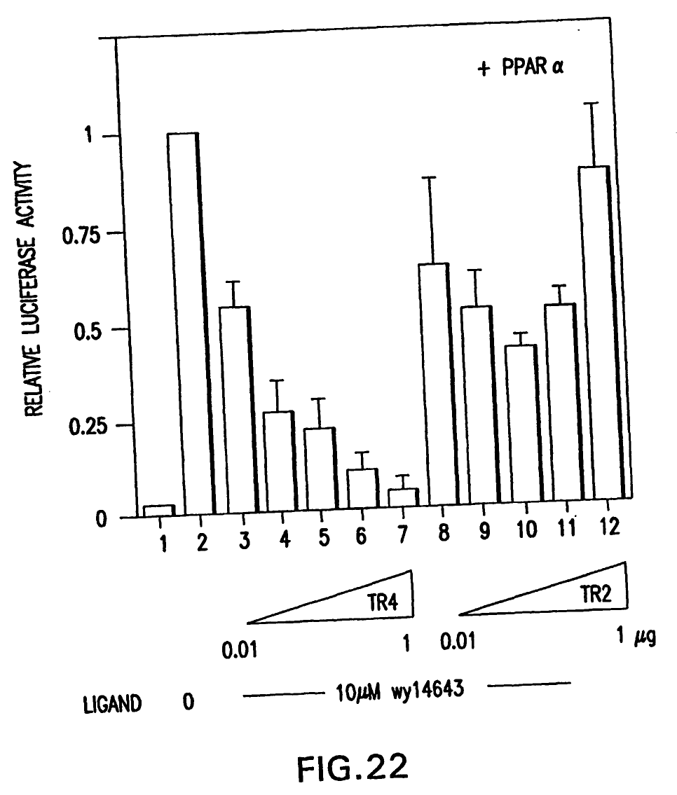

The effects of TR2 and TR4 on PPARα transactivation were examined by luciferase assays. Since our preliminary studies indicated that PPRE₃-luciferase activity without exogenous PPARα transfection is too low to detect, the PPARα expression vector pSG5-PPARα was transfected into HaCaT keratinocytes. As shown in FIG. 22, both TR2 and TR4 can suppress Wy-14643 (a PPARα-specific ligand)-induced PPARα transactivation. The suppression effects by TR4 and TR2, however, are quite different: only TR4 can repress PPARα signal pathway in a dose dependant manner that can reach a maximal of 95% suppression. In contrast, TR2 can first repress PPARα to 59.8% and then, surprisingly, the suppression gradually disappears with the addition of more TR2. (FIG. 22, lane 7). Together, these data clearly indicate that the PPARα signaling pathway can be differentially regulated by TR2 and TR4, two very close members in the orphan nuclear receptor superfamily.

### (6) Differential Binding Affinity of TR2 and TR4 Toward PPRE

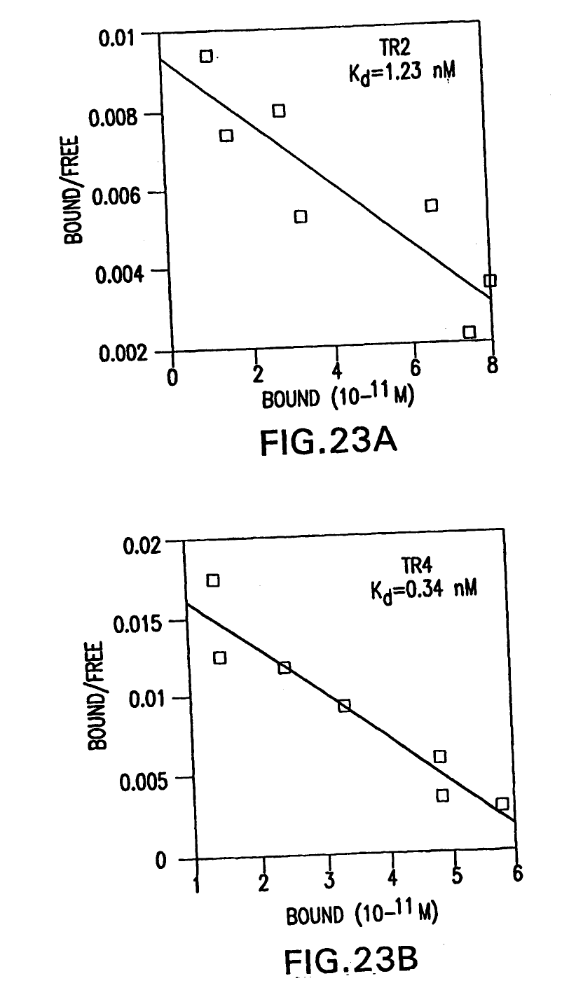

To clarify the potential mechanism for the differential regulation of PPARα signal pathway by TR2 and TR4, the Kds (equilibrium dissociation constants) for TR2 and TR4 binding to PPRE were calculated using Scatchard analysis. As shown in FIG. 23a and 23b, the Kds calculated from Scatchard plots for TR2 and TR4 were 1.23 nM and 0.34 nM, respectively, indicating that the affinity for TR4 is higher than that for TR2. This result may provide one explanation of how TR2 and TR4 can differentially repress the PPARα signal pathways. The Kd values calculated here were consistent with the range of Kd values for nuclear receptors and their hormone response elements. Together, these data clearly suggest that the PPARα signal pathway can be differentially regulated by TR2 and TR4, two very close members in the orphan nuclear receptor superfamily, in HaCaT keratinocytes.

### (7) Wy-14643 Upregulates the Expression Level of TR4 Protein But Downregulates that of TR2 Protein in HaCaT Keratinocytes

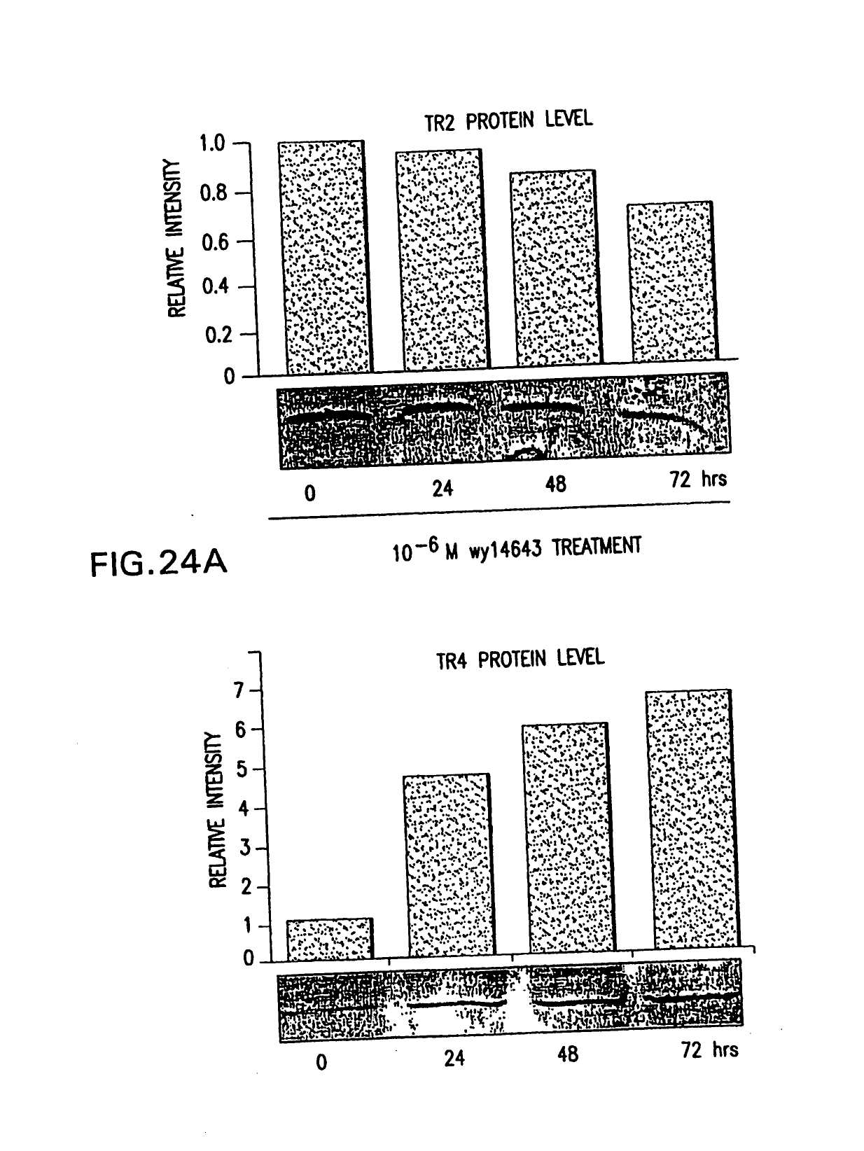

In order to investigate whether TR2 and TR4 can act as a negative feedback regulators, the effect of WY-14643 on the expression level of TR2 and TR4 protein using Western blot analysis. Following treatment with 10⁻⁵ M Wy-14643, TR4 protein expression increased approximately 4.58-, 5.80-, and 6.56-fold at 24, 48, and 72 hours, respectively, suggesting that TR4 can act as a strong negative feedback modulator for PPARα in human HaCaT keratinocytes (FIG. 24a). In contrast, TR2 decreased to 82% and 66% at 48 and 72 hours, respectively (FIG. 24b). These results indicate that TR2 and TR4 are differentially regulated by Wy-14643. As a control, 10⁻⁷ M 1,25-(OH)₂D₃ had no effect on TR2 and TR4 protein expression levels.

Comparison of amino acid sequence between TR2 and TR4 (Chang, C., and Kokontis, J. (1988) Biochem Biophys Res Commun 155, 971-977; Chang, C. et al. (1989) Biochem Biophys Res Commun 165, 735-741; Chang, C. et al. (1994) Proc Natl Acad Sci USA 91, 6040-6044) shows that these two orphan nuclear receptors share very high (64%) sequence homology. Their DNA-binding domains (DBD) have 81% homology with 100% homology in the P-box, a very important domain within the DBD to recognize the hormone response elements in the target genes. The DBD and P-box are well understood and readily identifiable. This high sequence homology between TR2 and TR4 is consistent with them having very similar biochemical and physiological function. TR2 and TR4 have very similar effects on the following signal pathways: 1) thyroid hormones (Lee, Y.-F. et al. (1997) J. Biol. Chem. 272, 12215-12220;), 2) ciliary neurotropic factor receptor (Young, W.-I. et al. (1997) J. Biol. Chem. 272, 3109-3116; Young, W.-J. et al. (1998) J. Biol. Chem. 273, 20877-20885), 3) R A (Lin, T.-M. et al. (1995) J. Biol. Chem. 270, 30121-30128; Lee, Y.-F. et al. (1998) J. Biol. Chem. 273, 13437-13443), 4) simian virus 40 gene expression (Lee, H.-J., and Chang, C. (1995) J. Biol. Chem. 270, 5434-5440; Lee, H.-J. et al. (1995) J. Biol. Chem. 270, 30129-30133), and 5) erythropoietin gene expression (Lee, H.-I. et al. (1996) J. Biol. Chem. 271, 10405-10412). Disclosed herein it is clearly demonstrated that TR2 and TR4 have distinct function by differential and bi-regulation in the PPARα signal pathway. This indicates that closely related members of the nuclear receptor superfamily, such as TR2 and TR4 can have distinct functional character.

To explore the potential mechanisms causing the differential regulation of TR2/4 and PPARα, the affinities of TR2 and TR4 binding to PPRE-DNA were measured. Scatchard analysis showed that the DNA-binding affinity of TR4 is higher than that of TR2, which is consistent with TR4 acting as a more sensitive modulator for PPARα or differential cofactor recruitment.

G. Sequences
1. SEQ ID NO:1. consensus DR A(G/T)(G/T)TCA)
2. SEQ ID NO:2 consensus DR4 A(G/T)(G/T)TCAccctc(G/T)(G/T)TCA.
3. SEQ ID NO:3 a DR4 ATGTCAccctAGTTCA
4. SEQ ID NO:4 a DR AGGTCA
5. SEQ ID NO:5 PPRE
6. SEQ ID NO:6 Human TR2 Protein Genbank accession number P13056.
7. SEQ ID NO:7 Genbank Accession no. A36738. orphan receptor TR2, splice form TR2-9—human.
8. SEQ ID NO:8 Genbank Accession No. B36738 orphan receptor TR2, splice form TR2-11—human.
9. SEQ ID NO:9 Genbank Accession No. A31521. orphan receptor TR2, splice form TR2-5—human.
10. SEQ ID NO:10 Genbank Accession No. 154075. gene mTR2R1 protein—mouse
11. SEQ ID NO:11 Genbank Accession No. M29959. Human steroid receptor (TR2-9) Protein.
12. SEQ ID NO:12 Genbank Accession No. M29959. Human steroid receptor (TR2-9) mRNA, complete cds. Encodes protein designated M29959 herein.
13. SEQ D NO:13 Genbank Accession No. M29960. Human steroid receptor (TR2-11) protein
14. SEQ ID NO:14 Genbank Accession No. M29960. Human steroid receptor (TR2-11) mRNA, complete cds encoding protein M299960 disclosed herein
15. SEQ ID NO:15 Genbank Accession No. M21985. Human steroid receptor TR2 protein.
16. SEQ ID NO:16 Genbank Accession No. M21985. Human steroid receptor TR2 mRNA, complete cds encodes protein disclosed herein as M21985
17. SEQ ID NO:17 Genbank Accession No P49116. Human Orphan nuclear receptor TR4 (Orphan nuclear receptor TAK1)
18. SEQ ID NO:18 Genbank Accession No. NP_{—}059019. Rat TR4 orphan receptor; orphan receptor, TR4 [Rattus norvegicus]
19. SEQ ID NO:19 I80177 TR4 orphan receptor—Rat
20. SEQ ID NO:20 I59309 TR4 orphan receptor—human
21. SEQ ID NO:21 TR4 nucleic acid Genbank Accession No: L27586. human TR4 orphan receptor protein
22. SEQ ID NO:22 TR4 nucleic acid Genbank Accession No: L27586. Human TR4 orphan receptor mRNA, complete cds encoding TR4 protein L27586
23. SEQ ID NO:23 Genbank Accession No. S80204 {L1-E6 region, long control region} [Human papillomavirus type 11],
24. SEQ ID NO:24 Genbank Accession No S80202. {L1-E6 region, long control region} [Human papillomavirus type 6a],
25. SEQ ID NO:25 Genbank Accession No: S80200. {L1-E6 region, long control region} [Human papillomavirus type 6b],
26. SEQ ID NO:26 Genbank Accession No. S60413. long control region: L1 orf, E6 orf {LCR microheterogeneity} [human
27. SEQ ID NO:27 Genbank Accession No. M12792. Homo sapiens CYP21 gene, exons 1 through 10; and steroid 21-hydroxylase (CYP21) gene, protein.

28. SEQ ID NO:28 Genbank Accession No. M12792. Homo sapiens CYP21 gene, exons 1 through 10; and steroid 21-hydroxylase (CYP21) gene, complete cds encodes protein disclosed herein, M12792

```
# SEQUENCE LIS
#TING
<160> NUMBER OF SEQ ID NOS: 28
<210> SEQ ID NO 1
<211> LENGTH: 6
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 1
akktca
#
#
# 6
<210> SEQ ID NO 2
<211> LENGTH: 16
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 2
akktcaccct akktca
#
#
# 16
<210> SEQ ID NO 3
<211> LENGTH: 16
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 3
atgtcaccct agttca
#
#
# 16
<210> SEQ ID NO 4
<211> LENGTH: 6
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 4
aggtca
#
#
# 6
<210> SEQ ID NO 5
<211> LENGTH: 42
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 5
gtcgacaggg gaccaggaca aaggtcacgt tcgggagtcg ac
#
# 42
<210> SEQ ID NO 6
<211> LENGTH: 483
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 6
Met Ala Thr Ile Glu Glu Ile Ala His Gln Il
#e Ile Glu Gln Gln Met
  1 5
# 10
# 15
Gly Glu Ile Val Thr Glu Gln Gln Thr Gly Gl
#n Lys Ile Gln Ile Val
#20
#25
#30
Thr Ala Leu Asp His Asn Thr Gln Gly Lys Gl
#n Phe Ile Leu Thr Asn
                35
# 40
# 45
His Asp Gly Ser Thr Pro Ser Lys Val Ile Le
#u Ala Arg Gln Asp Ser
        50
# 55
# 60
Thr Pro Gly Lys Val Phe Leu Thr Thr Pro As
#p Ala Ala Gly Val Asn
65
#70
#75
#80
Gln Leu Phe Phe Thr Thr Pro Asp Leu Ser Al
#a Gln His Leu Gln Leu
                                85
# 90
# 95
Leu Thr Asp Asn Ser Pro Asp Gln Gly Pro As
#n Lys Val Phe Asp Leu
                        100
# 105
# 110
Cys Val Val Cys Gly Asp Lys Ala Ser Gly Ar
#g His Tyr Gly Ala Val
                115
# 120
# 125
Thr Cys Glu Gly Cys Lys Gly Phe Phe Lys Ar
#g Ser Ile Arg Lys Asn
        130
# 135
# 140
Leu Val Tyr Ser Cys Arg Gly Ser Lys Asp Cy
#s Ile Ile Asn Lys His
145 1
#50 1
#55 1
#60
His Arg Asn Arg Cys Gln Tyr Cys Arg Leu Gl
#n Arg Cys Ile Ala Phe
                                165
# 170
# 175
Gly Met Lys Gln Asp Ser Val Gln Cys Glu Ar
#g Lys Pro Ile Glu Val
                        180
# 185
# 190
Ser Arg Glu Lys Ser Ser Asn Cys Ala Ala Se
#r Thr Glu Lys Ile Tyr
                195
# 200
# 205
Ile Arg Lys Asp Leu Arg Ser Pro Leu Thr Al
#a Thr Pro Thr Phe Val
        210
# 215
# 220
Thr Asp Ser Glu Ser Thr Arg Ser Thr Gly Le
#u Leu Asp Ser Gly Met
225 2
#30 2
#35 2
#40
Phe Met Asn Ile His Pro Ser Gly Val Lys Th
#r Glu Ser Ala Val Leu
                                245
# 250
# 255
Met Thr Ser Asp Lys Ala Glu Ser Cys Gln Gl
#y Asp Leu Ser Thr Leu
                        260
# 265
# 270
Ala Asn Val Val Thr Ser Leu Ala Asn Leu Gl
#y Lys Thr Lys Asp Leu
                275
# 280
# 285
Ser Gln Asn Ser Asn Glu Met Ser Met Ile Gl
#u Ser Leu Ser Asn Asp
        290
# 295
# 300
Asp Thr Ser Leu Cys Glu Phe Gln Glu Met Gl
#n Thr Asn Gly Asp Val
305 3
#10 3
#15 3
#20
Ser Arg Ala Phe Asp Thr Leu Ala Lys Ala Le
#u Asn Pro Gly Glu Ser
                                325
# 330
# 335
Thr Ala Cys Gln Ser Ser Val Ala Gly Met Gl
#u Gly Ser Val His Leu
                        340
# 345
# 350
Ile Thr Gly Asp Ser Ser Ile Asn Tyr Thr Gl
#u Lys Glu Gly Pro Leu
                355
# 360
# 365
Leu Ser Asp Ser His Val Ala Phe Arg Leu Th
#r Met Pro Ser Pro Met
        370
# 375
# 380
Pro Glu Tyr Leu Asn Val His Tyr Ile Gly Gl
#u Ser Ala Ser Arg Leu
385 3
#90 3
#95 4
#00
Leu Phe Leu Ser Met His Trp Ala Leu Ser Il
#e Pro Ser Phe Gln Ala
                                405
# 410
# 415
Leu Gly Gln Glu Asn Ser Ile Ser Leu Val Ly
#s Ala Tyr Trp Asn Glu
                        420
# 425
# 430
Leu Phe Thr Leu Gly Leu Ala Gln Cys Trp Gl
#n Val Met Asn Val Ala
                435
# 440
# 445
Thr Ile Leu Ala Thr Phe Val Asn Cys Leu Hi
#s Asn Ser Leu Gln Gln
        450
# 455
# 460
Asp Ala Lys Val Ile Ala Ala Leu Ile His Ph
#e Thr Arg Arg Ala Ile
465 4
#70 4
#75 4
#80
Thr Asp Leu
<210> SEQ ID NO 7
<211> LENGTH: 467
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 7
Met Ala Thr Ile Glu Glu Ile Ala His Gln Il
#e Ile Glu Gln Gln Met
  1 5
# 10
# 15
Gly Glu Ile Val Thr Glu Gln Gln Thr Gly Gl
#n Lys Ile Gln Ile Val
                        20
# 25
# 30
Thr Ala Leu Asp His Asn Thr Gln Gly Lys Gl
#n Phe Ile Leu Thr Asn
                35
# 40
# 45
His Asp Gly Ser Thr Pro Ser Lys Val Ile Le
#u Ala Arg Gln Asp Ser
        50
# 55
# 60
Thr Pro Gly Lys Val Phe Leu Thr Thr Pro As
#p Ala Ala Gly Val Asn
65
#70
#75
#80
Gln Leu Phe Phe Thr Thr Pro Asp Leu Ser Al
#a Gln His Leu Gln Leu
                                85
# 90
# 95
Leu Thr Asp Asn Ser Pro Asp Gln Gly Pro As
#n Lys Val Phe Asp Leu
                        100
# 105
# 110
Cys Val Val Cys Gly Asp Lys Ala Ser Gly Ar
#g His Tyr Gly Ala Val
                115
# 120
# 125
Thr Cys Glu Gly Cys Lys Gly Phe Phe Lys Ar
#g Ser Ile Arg Lys Asn
        130
# 135
# 140
Leu Val Tyr Ser Cys Arg Gly Ser Lys Asp Cy
#s Ile Ile Asn Lys His
145 1
#50 1
#55 1
#60
His Arg Asn Arg Cys Gln Tyr Cys Arg Leu Gl
#n Arg Cys Ile Ala Phe
                                165
# 170
# 175
Gly Met Lys Gln Asp Ser Val Gln Cys Glu Ar
#g Lys Pro Ile Glu Val
                        180
# 185
# 190
Ser Arg Glu Lys Ser Ser Asn Cys Ala Ala Se
#r Thr Glu Lys Ile Tyr
                195
# 200
# 205
Ile Arg Lys Asp Leu Arg Ser Pro Leu Thr Al
#a Thr Pro Thr Phe Val
        210
# 215
# 220
Thr Asp Ser Glu Ser Thr Arg Ser Thr Gly Le
#u Leu Asp Ser Gly Met
225 2
#30 2
#35 2
#40
Phe Met Asn Ile His Pro Ser Gly Val Lys Th
#r Glu Ser Ala Val Leu
                                245
# 250
# 255
Met Thr Ser Asp Lys Ala Glu Ser Cys Gln Gl
#y Asp Leu Ser Thr Leu
                        260
# 265
# 270
Ala Asn Val Val Thr Ser Leu Ala Asn Leu Gl
#y Lys Thr Lys Asp Leu
                275
# 280
# 285
Ser Gln Asn Ser Asn Glu Met Ser Met Ile Gl
#u Ser Leu Ser Asn Asp
        290
# 295
# 300
Asp Thr Ser Leu Cys Glu Phe Gln Glu Met Gl
#n Thr Asn Gly Asp Val
305 3
#10 3
#15 3
#20
Ser Arg Ala Phe Asp Thr Leu Ala Lys Ala Le
#u Asn Pro Gly Glu Ser
                                325
# 330
# 335
Thr Ala Cys Gln Ser Ser Val Ala Gly Met Gl
#u Gly Ser Val His Leu
                        340
# 345
# 350
Ile Thr Gly Asp Ser Ser Ile Asn Tyr Thr Gl
#u Lys Glu Gly Pro Leu
                355
# 360
# 365
Leu Ser Asp Ser His Val Ala Phe Arg Leu Th
#r Met Pro Ser Pro Met
        370
# 375
# 380
Pro Glu Tyr Leu Asn Val His Tyr Ile Gly Gl
#u Ser Ala Ser Arg Leu
385 3
#90 3
#95 4
#00
Leu Phe Leu Ser Met His Trp Ala Leu Ser Il
#e Pro Ser Phe Gln Ala
                                405
# 410
# 415
Leu Gly Gln Glu Asn Ser Ile Ser Leu Val Ly
#s Ala Tyr Trp Asn Glu
                        420
# 425
# 430
Leu Phe Thr Leu Gly Leu Ala Gln Cys Trp Gl
#n Val Met Asn Val Ala
                435
# 440
# 445
Thr Ile Leu Ala Thr Phe Val Asn Cys Leu Hi
#s Asn Ser Leu Gln Gln
        450
# 455
# 460
Ala Glu Gly
465
<210> SEQ ID NO 8
<211> LENGTH: 603
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 8
Met Ala Thr Ile Glu Glu Ile Ala His Gln Il
#e Ile Glu Gln Gln Met
  1 5
# 10
# 15
Gly Glu Ile Val Thr Glu Gln Gln Thr Gly Gl
#n Lys Ile Gln Ile Val
                        20
# 25
# 30
Thr Ala Leu Asp His Asn Thr Gln Gly Lys Gl
#n Phe Ile Leu Thr Asn
                35
# 40
# 45
His Asp Gly Ser Thr Pro Ser Lys Val Ile Le
#u Ala Arg Gln Asp Ser
        50
# 55
# 60
Thr Pro Gly Lys Val Phe Leu Thr Thr Pro As
#p Ala Ala Gly Val Asn
65
#70
#75
#80
Gln Leu Phe Phe Thr Thr Pro Asp Leu Ser Al
#a Gln His Leu Gln Leu
                                85
# 90
# 95
Leu Thr Asp Asn Ser Pro Asp Gln Gly Pro As
#n Lys Val Phe Asp Leu
                        100
# 105
# 110
Cys Val Val Cys Gly Asp Lys Ala Ser Gly Ar
#g His Tyr Gly Ala Val
                115
# 120
# 125
Thr Cys Glu Gly Cys Lys Gly Phe Phe Lys Ar
#g Ser Ile Arg Lys Asn
        130
# 135
# 140
Leu Val Tyr Ser Cys Arg Gly Ser Lys Asp Cy
#s Ile Ile Asn Lys His
145 1
#50 1
#55 1
#60
His Arg Asn Arg Cys Gln Tyr Cys Arg Leu Gl
#n Arg Cys Ile Ala Phe
                                165
# 170
# 175
Gly Met Lys Gln Asp Ser Val Gln Cys Glu Ar
#g Lys Pro Ile Glu Val
                        180
# 185
# 190
Ser Arg Glu Lys Ser Ser Asn Cys Ala Ala Se
#r Thr Glu Lys Ile Tyr
                195
# 200
# 205
Ile Arg Lys Asp Leu Arg Ser Pro Leu Thr Al
#a Thr Pro Thr Phe Val
        210
# 215
# 220
Thr Asp Ser Glu Ser Thr Arg Ser Thr Gly Le
#u Leu Asp Ser Gly Met
225 2
#30 2
#35 2
#40
Phe Met Asn Ile His Pro Ser Gly Val Lys Th
#r Glu Ser Ala Val Leu
                                245
# 250
# 255
Met Thr Ser Asp Lys Ala Glu Ser Cys Gln Gl
#y Asp Leu Ser Thr Leu
                        260
# 265
# 270
Ala Asn Val Val Thr Ser Leu Ala Asn Leu Gl
#y Lys Thr Lys Asp Leu
                275
# 280
# 285
Ser Gln Asn Ser Asn Glu Met Ser Met Ile Gl
#u Ser Leu Ser Asn Asp
        290
# 295
# 300
Asp Thr Ser Leu Cys Glu Phe Gln Glu Met Gl
#n Thr Asn Gly Asp Val
305 3
#10 3
#15 3
#20
Ser Arg Ala Phe Asp Thr Leu Ala Lys Ala Le
#u Asn Pro Gly Glu Ser
                                325
# 330
# 335
Thr Ala Cys Gln Ser Ser Val Ala Gly Met Gl
#u Gly Ser Val His Leu
                        340
# 345
# 350
Ile Thr Gly Asp Ser Ser Ile Asn Tyr Thr Gl
#u Lys Glu Gly Pro Leu
                355
# 360
# 365
Leu Ser Asp Ser His Val Ala Phe Arg Leu Th
#r Met Pro Ser Pro Met
        370
# 375
# 380
Pro Glu Tyr Leu Asn Val His Tyr Ile Gly Gl
#u Ser Ala Ser Arg Leu
385 3
#90 3
#95 4
#00
Leu Phe Leu Ser Met His Trp Ala Leu Ser Il
#e Pro Ser Phe Gln Ala
                                405
# 410
# 415
Leu Gly Gln Glu Asn Ser Ile Ser Leu Val Ly
#s Ala Tyr Trp Asn Glu
                        420
# 425
# 430
Leu Phe Thr Leu Gly Leu Ala Gln Cys Trp Gl
#n Val Met Asn Val Ala
                435
# 440
# 445
Thr Ile Leu Ala Thr Phe Val Asn Cys Leu Hi
#s Asn Ser Leu Gln Gln
        450
# 455
# 460
Asp Lys Met Ser Thr Glu Arg Arg Lys Leu Le
#u Met Glu His Ile Phe
465 4
#70 4
#75 4
#80
Lys Leu Gln Glu Phe Cys Asn Ser Met Val Ly
#s Leu Cys Ile Asp Gly
                                485
# 490
# 495
Tyr Glu Tyr Ala Tyr Leu Lys Ala Ile Val Le
#u Phe Ser Pro Asp His
                        500
# 505
# 510
Pro Ser Leu Glu Asn Met Glu Leu Ile Glu Ly
#s Phe Gln Glu Lys Ala
                515
# 520
# 525
Tyr Val Glu Phe Gln Asp Tyr Ile Thr Lys Th
#r Tyr Pro Asp Asp Thr
        530
# 535
# 540
Tyr Arg Leu Ser Arg Leu Leu Leu Arg Leu Pr
#o Ala Leu Arg Leu Met
545 5
#50 5
#55 5
#60
Asn Ala Thr Ile Thr Glu Glu Leu Phe Phe Ly
#s Gly Leu Ile Gly Asn
                                565
# 570
# 575
Ile Arg Ile Asp Ser Val Ile Pro His Ile Le
#u Lys Met Glu Pro Ala
                        580
# 585
# 590
Asp Tyr Asn Ser Gln Ile Ile Gly His Ile Se
#r
                595
# 600
<210> SEQ ID NO 9
<211> LENGTH: 483
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 9
Met Ala Thr Ile Glu Glu Ile Ala His Gln Il
#e Ile Glu Gln Gln Met
  1 5
# 10
# 15
Gly Glu Ile Val Thr Glu Gln Gln Thr Gly Gl
#n Lys Ile Gln Ile Val
                        20
# 25
# 30
Thr Ala Leu Asp His Asn Thr Gln Gly Lys Gl
#n Phe Ile Leu Thr Asn
                35
# 40
# 45
His Asp Gly Ser Thr Pro Ser Lys Val Ile Le
#u Ala Arg Gln Asp Ser
        50
# 55
# 60
Thr Pro Gly Lys Val Phe Leu Thr Thr Pro As
#p Ala Ala Gly Val Asn
65
#70
#75
#80
Gln Leu Phe Phe Thr Thr Pro Asp Leu Ser Al
#a Gln His Leu Gln Leu
                                85
# 90
# 95
Leu Thr Asp Asn Ser Pro Asp Gln Gly Pro As
#n Lys Val Phe Asp Leu
                        100
# 105
# 110
Cys Val Val Cys Gly Asp Lys Ala Ser Gly Ar
#g His Tyr Gly Ala Val
                115
# 120
# 125
Thr Cys Glu Gly Cys Lys Gly Phe Phe Lys Ar
#g Ser Ile Arg Lys Asn
        130
# 135
# 140
Leu Val Tyr Ser Cys Arg Gly Ser Lys Asp Cy
#s Ile Ile Asn Lys His
145 1
#50 1
#55 1
#60
His Arg Asn Arg Cys Gln Tyr Cys Arg Leu Gl
#n Arg Cys Ile Ala Phe
                                165
# 170
# 175
Gly Met Lys Gln Asp Ser Val Gln Cys Glu Ar
#g Lys Pro Ile Glu Val
                        180
# 185
# 190
Ser Arg Glu Lys Ser Ser Asn Cys Ala Ala Se
#r Thr Glu Lys Ile Tyr
                195
# 200
# 205
Ile Arg Lys Asp Leu Arg Ser Pro Leu Thr Al
#a Thr Pro Thr Phe Val
        210
# 215
# 220
Thr Asp Ser Glu Ser Thr Arg Ser Thr Gly Le
#u Leu Asp Ser Gly Met
225 2
#30 2
#35 2
#40
Phe Met Asn Ile His Pro Ser Gly Val Lys Th
#r Glu Ser Ala Val Leu
                                245
# 250
# 255
Met Thr Ser Asp Lys Ala Glu Ser Cys Gln Gl
#y Asp Leu Ser Thr Leu
                        260
# 265
# 270
Ala Asn Val Val Thr Ser Leu Ala Asn Leu Gl
#y Lys Thr Lys Asp Leu
                275
# 280
# 285
Ser Gln Asn Ser Asn Glu Met Ser Met Ile Gl
#u Ser Leu Ser Asn Asp
        290
# 295
# 300
Asp Thr Ser Leu Cys Glu Phe Gln Glu Met Gl
#n Thr Asn Gly Asp Val
305 3
#10 3
#15 3
#20
Ser Arg Ala Phe Asp Thr Leu Ala Lys Ala Le
#u Asn Pro Gly Glu Ser
                                325
# 330
# 335
Thr Ala Cys Gln Ser Ser Val Ala Gly Met Gl
#u Gly Ser Val His Leu
                        340
# 345
# 350
Ile Thr Gly Asp Ser Ser Ile Asn Tyr Thr Gl
#u Lys Glu Gly Pro Leu
                355
# 360
# 365
Leu Ser Asp Ser His Val Ala Phe Arg Leu Th
#r Met Pro Ser Pro Met
        370
# 375
# 380
Pro Glu Tyr Leu Asn Val His Tyr Ile Gly Gl
#u Ser Ala Ser Arg Leu
385 3
#90 3
#95 4
#00
Leu Phe Leu Ser Met His Trp Ala Leu Ser Il
#e Pro Ser Phe Gln Ala
                                405
# 410
# 415
Leu Gly Gln Glu Asn Ser Ile Ser Leu Val Ly
#s Ala Tyr Trp Asn Glu
                        420
# 425
# 430
Leu Phe Thr Leu Gly Leu Ala Gln Cys Trp Gl
#n Val Met Asn Val Ala
                435
# 440
# 445
Thr Ile Leu Ala Thr Phe Val Asn Cys Leu Hi
#s Asn Ser Leu Gln Gln
        450
# 455
# 460
Asp Ala Lys Val Ile Ala Ala Leu Ile His Ph
#e Thr Arg Arg Ala Ile
465 4
#70 4
#75 4
#80
Thr Asp Leu
<210> SEQ ID NO 10
<211> LENGTH: 629
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 10
Met Ala Thr Asn Met Glu Gly Leu Val Gln Hi
#s Arg Val Gly Thr Gln
  1 5
# 10
# 15
Gln Val Ala Glu Val Pro Arg Thr Gln Thr Se
#r Trp Pro Glu Ser Pro
                        20
# 25
# 30
Gly Met Thr Ser Pro Ser Pro Arg Ile Gln Il
#e Ile Ser Thr Asp Ser
                35
# 40
# 45
Ala Val Ala Ser Pro Gln Arg Ile Gln Ile Va
#l Thr Asp Gln Gln Thr
        50
# 55
# 60
Gly Gln Lys Ile Gln Ile Val Thr Ala Val As
#p Ala Ser Gly Ser Ser
65
#70
#75
#80
Lys Gln Gln Phe Ile Leu Thr Ser Pro Asp Gl
#y Ala Gly Thr Gly Lys
                                85
# 90
# 95
Val Ile Leu Ala Ser Pro Glu Thr Ser Ser Al
#a Lys Gln Leu Ile Phe
                        100
# 105
# 110
Thr Thr Ser Asp Asn Leu Val Pro Gly Arg Il
#e Gln Ile Val Thr Asp
                115
# 120
# 125
Ser Ala Ser Val Glu Arg Leu Leu Gly Lys Al
#a Asp Val Gln Arg Pro
        130
# 135
# 140
Gln Val Val Glu Tyr Cys Val Val Cys Gly As
#p Lys Ala Ser Gly Arg
145 1
#50 1
#55 1
#60
His Tyr Gly Ala Val Ser Cys Glu Gly Cys Ly
#s Gly Phe Phe Lys Arg
                                165
# 170
# 175
Ser Val Arg Lys Asn Leu Thr Tyr Ser Cys Ar
#g Ser Ser Gln Asp Cys
                        180
# 185
# 190
Ile Ile Asn Lys His His Arg Asn Arg Cys Gl
#n Phe Cys Arg Leu Lys
                195
# 200
# 205
Lys Cys Leu Glu Met Gly Met Lys Met Glu Se
#r Val Gln Ser Glu Arg
        210
# 215
# 220
Lys Pro Phe Asp Val Gln Arg Glu Lys Pro Se
#r Asn Cys Ala Ala Ser
225 2
#30 2
#35 2
#40
Thr Glu Lys Ile Tyr Ile Arg Lys Asp Leu Ar
#g Ser Pro Leu Ile Ala
                                245
# 250
# 255
Thr Pro Thr Phe Val Ala Asp Lys Asp Gly Al
#a Arg Gln Thr Gly Leu
                        260
# 265
# 270
Leu Asp Pro Gly Met Leu Val Asn Ile Gln Gl
#n Pro Leu Ile Arg Glu
                275
# 280
# 285
Asp Gly Thr Val Leu Leu Ala Ala Asp Ser Ly
#s Ala Glu Thr Ser Gln
        290
# 295
# 300
Gly Ala Leu Gly Thr Leu Ala Asn Val Val Th
#r Ser Leu Ala Asn Leu
305 3
#10 3
#15 3
#20
Ser Glu Ser Leu Asn Asn Gly Asp Ala Ser Gl
#u Met Gln Pro Glu Asp
                                325
# 330
# 335
Gln Ser Ala Ser Glu Ile Thr Arg Ala Phe As
#p Thr Leu Ala Lys Ala
                        340
# 345
# 350
Leu Asn Thr Thr Asp Ser Ala Ser Pro Pro Se
#r Leu Ala Asp Gly Ile
                355
# 360
# 365
Asp Ala Ser Gly Gly Gly Ser Ile His Val Il
#e Ser Arg Asp Gln Ser
        370
# 375
# 380
Thr Pro Ile Ile Glu Val Glu Gly Pro Leu Le
#u Ser Asp Thr His Val
385 3
#90 3
#95 4
#00
Thr Phe Lys Leu Thr Met Pro Ser Pro Met Pr
#o Glu Tyr Leu Asn Val
                                405
# 410
# 415
His Tyr Ile Cys Glu Ser Ala Ser Arg Leu Le
#u Phe Leu Ser Met His
                        420
# 425
# 430
Trp Ala Arg Ser Ile Pro Ala Phe Gln Ala Le
#u Gly Gln Asp Cys Asn
                435
# 440
# 445
Thr Ser Leu Val Arg Ala Cys Trp Asn Glu Le
#u Phe Thr Leu Gly Leu
        450
# 455
# 460
Ala Gln Cys Ala Gln Val Met Ser Leu Ser Th
#r Ile Leu Ala Ala Ile
465 4
#70 4
#75 4
#80
Val Asn His Leu Gln Asn Ser Ile Gln Glu As
#p Lys Leu Ser Gly Asp
                                485
# 490
# 495
Arg Ile Lys Gln Val Met Glu His Ile Trp Ly
#s Leu Gln Glu Phe Cys
                        500
# 505
# 510
Asn Ser Met Ala Lys Leu Asp Ile Asp Gly Ty
#r Glu Tyr Ala Tyr Leu
                515
# 520
# 525
Lys Ala Ile Val Leu Phe Ser Pro Asp His Pr
#o Gly Leu Thr Gly Thr
        530
# 535
# 540
Ser Gln Ile Glu Lys Phe Gln Glu Lys Ala Gl
#n Met Glu Leu Gln Asp
545 5
#50 5
#55 5
#60
Tyr Val Gln Lys Thr Tyr Ser Glu Asp Thr Ty
#r Arg Leu Ala Arg Ile
                                565
# 570
# 575
Leu Val Arg Leu Pro Ala Leu Arg Leu Met Se
#r Ser Asn Ile Thr Glu
                        580
# 585
# 590
Glu Leu Phe Phe Thr Gly Leu Ile Gly Asn Va
#l Ser Ile Asp Ser Ile
                595
# 600
# 605
Ile Pro Tyr Ile Leu Lys Met Glu Thr Ala Gl
#u Tyr Asn Gly Gln Ile
        610
# 615
# 620
Thr Gly Ala Ser Leu
625
<210> SEQ ID NO 11
<211> LENGTH: 467
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 11
Met Ala Thr Ile Glu Glu Ile Ala His Gln Il
#e Ile Glu Gln Gln Met
  1 5
# 10
# 15
Gly Glu Ile Val Thr Glu Gln Gln Thr Gly Gl
#n Lys Ile Gln Ile Val
                        20
# 25
# 30
Thr Ala Leu Asp His Asn Thr Gln Gly Lys Gl
#n Phe Ile Leu Thr Asn
                35
# 40
# 45
His Asp Gly Ser Thr Pro Ser Lys Val Ile Le
#u Ala Arg Gln Asp Ser
        50
# 55
# 60
Thr Pro Gly Lys Val Phe Leu Thr Thr Pro As
#p Ala Ala Gly Val Asn
65
#70
#75
#80
Gln Leu Phe Phe Thr Thr Pro Asp Leu Ser Al
#a Gln His Leu Gln Leu
                                85
# 90
# 95
Leu Thr Asp Asn Ser Pro Asp Gln Gly Pro As
#n Lys Val Phe Asp Leu
                        100
# 105
# 110
Cys Val Val Cys Gly Asp Lys Ala Ser Gly Ar
#g His Tyr Gly Ala Val
                115
# 120
# 125
Thr Cys Glu Gly Cys Lys Gly Phe Phe Lys Ar
#g Ser Ile Arg Lys Asn
        130
# 135
# 140
Leu Val Tyr Ser Cys Arg Gly Ser Lys Asp Cy
#s Ile Ile Asn Lys His
145 1
#50 1
#55 1
#60
His Arg Asn Arg Cys Gln Tyr Cys Arg Leu Gl
#n Arg Cys Ile Ala Phe
                                165
# 170
# 175
Gly Met Lys Gln Asp Ser Val Gln Cys Glu Ar
#g Lys Pro Ile Glu Val
                        180
# 185
# 190
Ser Arg Glu Lys Ser Ser Asn Cys Ala Ala Se
#r Thr Glu Lys Ile Tyr
                195
# 200
# 205
Ile Arg Lys Asp Leu Arg Ser Pro Leu Thr Al
#a Thr Pro Thr Phe Val
        210
# 215
# 220
Thr Asp Ser Glu Ser Thr Arg Ser Thr Gly Le
#u Leu Asp Ser Gly Met
225 2
#30 2
#35 2
#40
Phe Met Asn Ile His Pro Ser Gly Val Lys Th
#r Glu Ser Ala Val Leu
                                245
# 250
# 255
Met Thr Ser Asp Lys Ala Glu Ser Cys Gln Gl
#y Asp Leu Ser Thr Leu
                        260
# 265
# 270
Ala Asn Val Val Thr Ser Leu Ala Asn Leu Gl
#y Lys Thr Lys Asp Leu
                275
# 280
# 285
Ser Gln Asn Ser Asn Glu Met Ser Met Ile Gl
#u Ser Leu Ser Asn Asp
        290
# 295
# 300
Asp Thr Ser Leu Cys Glu Phe Gln Glu Met Gl
#n Thr Asn Gly Asp Val
305 3
#10 3
#15 3
#20
Ser Arg Ala Phe Asp Thr Leu Ala Lys Ala Le
#u Asn Pro Gly Glu Ser
                                325
# 330
# 335
Thr Ala Cys Gln Ser Ser Val Ala Gly Met Gl
#u Gly Ser Val His Leu
                        340
# 345
# 350
Ile Thr Gly Asp Ser Ser Ile Asn Tyr Thr Gl
#u Lys Glu Gly Pro Leu
                355
# 360
# 365
Leu Ser Asp Ser His Val Ala Phe Arg Leu Th
#r Met Pro Ser Pro Met
        370
# 375
# 380
Pro Glu Tyr Leu Asn Val His Tyr Ile Gly Gl
#u Ser Ala Ser Arg Leu
385 3
#90 3
#95 4
#00
Leu Phe Leu Ser Met His Trp Ala Leu Ser Il
#e Pro Ser Phe Gln Ala
                                405
# 410
# 415
Leu Gly Gln Glu Asn Ser Ile Ser Leu Val Ly
#s Ala Tyr Trp Asn Glu
                        420
# 425
# 430
Leu Phe Thr Leu Gly Leu Ala Gln Cys Trp Gl
#n Val Met Asn Val Ala
                435
# 440
# 445
Thr Ile Leu Ala Thr Phe Val Asn Cys Leu Hi
#s Asn Ser Leu Gln Gln
        450
# 455
# 460
Ala Glu Gly
465
<210> SEQ ID NO 12
<211> LENGTH: 1785
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 12
gaattcgggc ccgtcggctt tcttcaaccc tctcttcccg gagcgccccc aa
#tccacgag 60
tggcagccgc gggactgtcg cgtcggcgcc cgacgcggag tcagcagggg cg
#aaaagcgg 120
tagatcatgg caaccataga agaaattgca catcaaatta ttgaacaaca ga
#tgggagag 180
attgttacag agcagcaaac tgggcagaaa atccagattg tgacagcact tg
#atcataat 240
acccaaggca agcagttcat tctgacaaat cacgacggct ctactccaag ca
#aagtcatt 300
ctggccaggc aagattccac tccgggaaaa gttttcctta caactccaga tg
#cagcaggt 360
gtcaaccagt tattttttac cactcctgat ctgtctgcac aacacctgca gc
#tcctaaca 420
gataattctc cagaccaagg accaaataag gtttttgatc tttgcgtagt at
#gtggagac 480
aaagcatcag gacgtcatta tggagcagta acttgtgaag gctgcaaagg at
#tttttaaa 540
agaagcatcc gaaaaaattt agtatattca tgtcgaggat caaaggattg ta
#ttattaat 600
aagcaccacc gaaaccgctg tcaatactgc aggttacaga gatgtattgc gt
#ttggaatg 660
aagcaagact ctgtccaatg tgaaagaaaa cccattgaag tatcacgaga aa
#aatcttcc 720
aactgtgccg cttcaacaga aaaaatctat atccgaaagg accttcgtag cc
#cattaact 780
gcaactccaa cttttgtaac agatagtgaa agtacaaggt caacaggact gt
#tagattca 840
ggaatgttca tgaatattca tccatctgga gtaaaaactg agtcagctgt gc
#tgatgaca 900
tcagataagg ctgaatcatg tcagggagat ttaagtacat tggccaatgt gg
#ttacatca 960
ttagcgaatc ttggaaaaac taaagatctt tctcaaaata gtaatgaaat gt
#ctatgatt 1020
gaaagcttaa gcaatgatga tacctctttg tgtgaatttc aagaaatgca ga
#ccaacggt 1080
gatgtttcaa gggcatttga cactcttgca aaagcattga atcctggaga ga
#gcacagcc 1140
tgccagagct cagtagcggg catggaagga agtgtacacc taatcactgg ag
#attcaagc 1200
ataaattaca ccgaaaaaga ggggccactt ctcagcgatt cacatgtagc tt
#tcaggctc 1260
accatgcctt ctcctatgcc tgagtacctg aatgtgcact acattgggga gt
#ctgcctcc 1320
agactgctgt tcttatcaat gcactgggca ctttcgattc cttctttcca gg
#ctctaggg 1380
caagaaaaca gcatatcact ggtgaaagct tactggaatg aactttttac tc
#ttggtctt 1440
gcccagtgct ggcaagtgat gaatgtagca actatattag caacatttgt ca
#attgtctt 1500
cacaatagtc ttcaacaagc agaggggtaa tcaccttaaa atgtcatcaa aa
#atagatct 1560
actagaaggc agcatcacat tcccatctta cttatggact cctacccctg gt
#tcatgtct 1620
tatatgcctg taatggttat aaagcctacc ttcaggaaag ctatggttga ct
#aattacta 1680
atggatgggt tttaaacatg tccctctaca ataaattaaa atctttcaat gt
#ttgaatat 1740
aatgtggagg tgtttacctg agggcctctc tatctccccg aattc
# 1785
<210> SEQ ID NO 13
<211> LENGTH: 603
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 13
Met Ala Thr Ile Glu Glu Ile Ala His Gln Il
#e Ile Glu Gln Gln Met
  1 5
# 10
# 15
Gly Glu Ile Val Thr Glu Gln Gln Thr Gly Gl
#n Lys Ile Gln Ile Val
                        20
# 25
# 30
Thr Ala Leu Asp His Asn Thr Gln Gly Lys Gl
#n Phe Ile Leu Thr Asn
                35
# 40
# 45
His Asp Gly Ser Thr Pro Ser Lys Val Ile Le
#u Ala Arg Gln Asp Ser
        50
# 55
# 60
Thr Pro Gly Lys Val Phe Leu Thr Thr Pro As
#p Ala Ala Gly Val Asn
65
#70
#75
#80
Gln Leu Phe Phe Thr Thr Pro Asp Leu Ser Al
#a Gln His Leu Gln Leu
                                85
# 90
# 95
Leu Thr Asp Asn Ser Pro Asp Gln Gly Pro As
#n Lys Val Phe Asp Leu
                        100
# 105
# 110
Cys Val Val Cys Gly Asp Lys Ala Ser Gly Ar
#g His Tyr Gly Ala Val
                115
# 120
# 125
Thr Cys Glu Gly Cys Lys Gly Phe Phe Lys Ar
#g Ser Ile Arg Lys Asn
        130
# 135
# 140
Leu Val Tyr Ser Cys Arg Gly Ser Lys Asp Cy
#s Ile Ile Asn Lys His
145 1
#50 1
#55 1
#60
His Arg Asn Arg Cys Gln Tyr Cys Arg Leu Gl
#n Arg Cys Ile Ala Phe
                                165
# 170
# 175
Gly Met Lys Gln Asp Ser Val Gln Cys Glu Ar
#g Lys Pro Ile Glu Val
                        180
# 185
# 190
Ser Arg Glu Lys Ser Ser Asn Cys Ala Ala Se
#r Thr Glu Lys Ile Tyr
                195
# 200
# 205
Ile Arg Lys Asp Leu Arg Ser Pro Leu Thr Al
#a Thr Pro Thr Phe Val
        210
# 215
# 220
Thr Asp Ser Glu Ser Thr Arg Ser Thr Gly Le
#u Leu Asp Ser Gly Met
225 2
#30 2
#35 2
#40
Phe Met Asn Ile His Pro Ser Gly Val Lys Th
#r Glu Ser Ala Val Leu
                                245
# 250
# 255
Met Thr Ser Asp Lys Ala Glu Ser Cys Gln Gl
#y Asp Leu Ser Thr Leu
                        260
# 265
# 270
Ala Asn Val Val Thr Ser Leu Ala Asn Leu Gl
#y Lys Thr Lys Asp Leu
                275
# 280
# 285
Ser Gln Asn Ser Asn Glu Met Ser Met Ile Gl
#u Ser Leu Ser Asn Asp
        290
# 295
# 300
Asp Thr Ser Leu Cys Glu Phe Gln Glu Met Gl
#n Thr Asn Gly Asp Val
305 3
#10 3
#15 3
#20
Ser Arg Ala Phe Asp Thr Leu Ala Lys Ala Le
#u Asn Pro Gly Glu Ser
                                325
# 330
# 335
Thr Ala Cys Gln Ser Ser Val Ala Gly Met Gl
#u Gly Ser Val His Leu
                        340
# 345
# 350
Ile Thr Gly Asp Ser Ser Ile Asn Tyr Thr Gl
#u Lys Glu Gly Pro Leu
                355
# 360
# 365
Leu Ser Asp Ser His Val Ala Phe Arg Leu Th
#r Met Pro Ser Pro Met
        370
# 375
# 380
Pro Glu Tyr Leu Asn Val His Tyr Ile Gly Gl
#u Ser Ala Ser Arg Leu
385 3
#90 3
#95 4
#00
Leu Phe Leu Ser Met His Trp Ala Leu Ser Il
#e Pro Ser Phe Gln Ala
                                405
# 410
# 415
Leu Gly Gln Glu Asn Ser Ile Ser Leu Val Ly
#s Ala Tyr Trp Asn Glu
                        420
# 425
# 430
Leu Phe Thr Leu Gly Leu Ala Gln Cys Trp Gl
#n Val Met Asn Val Ala
                435
# 440
# 445
Thr Ile Leu Ala Thr Phe Val Asn Cys Leu Hi
#s Asn Ser Leu Gln Gln
        450
# 455
# 460
Asp Lys Met Ser Thr Glu Arg Arg Lys Leu Le
#u Met Glu His Ile Phe
465 4
#70 4
#75 4
#80
Lys Leu Gln Glu Phe Cys Asn Ser Met Val Ly
#s Leu Cys Ile Asp Gly
                                485
# 490
# 495
Tyr Glu Tyr Ala Tyr Leu Lys Ala Ile Val Le
#u Phe Ser Pro Asp His
                        500
# 505
# 510
Pro Ser Leu Glu Asn Met Glu Leu Ile Glu Ly
#s Phe Gln Glu Lys Ala
                515
# 520
# 525
Tyr Val Glu Phe Gln Asp Tyr Ile Thr Lys Th
#r Tyr Pro Asp Asp Thr
        530
# 535
# 540
Tyr Arg Leu Ser Arg Leu Leu Leu Arg Leu Pr
#o Ala Leu Arg Leu Met
545 5
#50 5
#55 5
#60
Asn Ala Thr Ile Thr Glu Glu Leu Phe Phe Ly
#s Gly Leu Ile Gly Asn
                                565
# 570
# 575
Ile Arg Ile Asp Ser Val Ile Pro His Ile Le
#u Lys Met Glu Pro Ala
                        580
# 585
# 590
Asp Tyr Asn Ser Gln Ile Ile Gly His Ser Il
#e
                595
# 600
<210> SEQ ID NO 14
<211> LENGTH: 2202
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 14
gggactgtcg cgtcggcgcc cgacgcggag tcagcagggg cgaaaagcgg ta
#gatcatgg 60
caaccataga agaaattgca catcaaatta ttgaacaaca gatgggagag at
#tgttacag 120
agcagcaaac tgggcagaaa atccagattg tgacagcact tgatcataat ac
#ccaaggca 180
agcagttcat tctgacaaat cacgacggct ctactccaag caaagtcatt ct
#ggccaggc 240
aagattccac tccgggaaaa gttttcctta caactccaga tgcagcaggt gt
#caaccagt 300
tattttttac cactcctgat ctgtctgcac aacacctgca gctcctaaca ga
#taattctc 360
cagaccaagg accaaataag gtttttgatc tttgcgtagt atgtggagac aa
#agcatcag 420
gacgtcatta tggagcagta acttgtgaag gctgcaaagg attttttaaa ag
#aagcatcc 480
gaaaaaattt agtatattca tgtcgaggat caaaggattg tattattaat aa
#gcaccacc 540
gaaaccgctg tcaatactgc aggttacaga gatgtattgc gtttggaatg aa
#gcaagact 600
ctgtccaatg tgaaagaaaa cccattgaag tatcacgaga aaaatcttcc aa
#ctgtgccg 660
cttcaacaga aaaaatctat atccgaaagg accttcgtag cccattaact gc
#aactccaa 720
cttttgtaac agatagtgaa agtacaaggt caacaggact gttagattca gg
#aatgttca 780
tgaatattca tccatctgga gtaaaaactg agtcagctgt gctgatgaca tc
#agataagg 840
ctgaatcatg tcagggagat ttaagtacat tggccaatgt ggttacatca tt
#agcgaatc 900
ttggaaaaac taaagatctt tctcaaaata gtaatgaaat gtctatgatt ga
#aagcttaa 960
gcaatgatga tacctctttg tgtgaatttc aagaaatgca gaccaacggt ga
#tgtttcaa 1020
gggcatttga cactcttgca aaagcattga atcctggaga gagcacagcc tg
#ccagagct 1080
cagtagcggg catggaagga agtgtacacc taatcactgg agattcaagc at
#aaattaca 1140
ccgaaaaaga ggggccactt ctcagcgatt cacatgtagc tttcaggctc ac
#catgcctt 1200
ctcctatgcc tgagtacctg aatgtgcact acattgggga gtctgcctcc ag
#actgctgt 1260
tcttatcaat gcactgggca ctttcgattc cttctttcca ggctctaggg ca
#agaaaaca 1320
gcatatcact ggtgaaagct tactggaatg aactttttac tcttggtctt gc
#ccagtgct 1380
ggcaagtgat gaatgtagca actatattag caacatttgt caattgtctt ca
#caatagtc 1440
ttcaacaaga taaaatgtca acagaaagaa gaaaattatt gatggagcac at
#cttcaaac 1500
tacaggagtt ttgtaacagc atggttaaac tctgcattga tggatacgaa ta
#tgcctacc 1560
tgaaggcaat agtactcttc agtccagatc atccaagcct agaaaacatg ga
#actgatag 1620
agaaatttca ggaaaaggct tatgtggaat tccaagatta tataaccaaa ac
#atatccag 1680
atgacaccta caggttatcc agactactac tcagattgcc agctttaaga ct
#gatgaatg 1740
ctaccatcac tgaagaattg tttttcaaag gtctcattgg caatatacga at
#tgacagtg 1800
ttatcccaca tattttgaaa atggagcctg cagattataa ctctcaaata at
#tggtcaca 1860
gcatttgaaa actgtgactg cagtgctgta aacttaactg ttctttgcca ga
#acacaaga 1920
caccaaattg aactcactgc ttttgaggca tctggaaatt tttactttaa aa
#agtaacca 1980
gaatccaagg tatttttatt ttagcttccc ttaagaattt ttgaagtgac tg
#ggcaggca 2040
gcagaaatta aatgaatttt tcttcctgat tcctttaaat gaatatgaaa ca
#ctacaaat 2100
ttattcttgg tgaagatgat acctgaagct gtcacctctt gattatctaa ac
#taagcgct 2160
cattctattt tataaaacaa ataaattagt ctcttttttc tg
#
#2202
<210> SEQ ID NO 15
<211> LENGTH: 483
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 15
Met Ala Thr Ile Glu Glu Ile Ala His Gln Il
#e Ile Glu Gln Gln Met
  1 5
# 10
# 15
Gly Glu Ile Val Thr Glu Gln Gln Thr Gly Gl
#n Lys Ile Gln Ile Val
                        20
# 25
# 30
Thr Ala Leu Asp His Asn Thr Gln Gly Lys Gl
#n Phe Ile Leu Thr Asn
                35
# 40
# 45
His Asp Gly Ser Thr Pro Ser Lys Val Ile Le
#u Ala Arg Gln Asp Ser
        50
# 55
# 60
Thr Pro Gly Lys Val Phe Leu Thr Thr Pro As
#p Ala Ala Gly Val Asn
65
#70
#75
#80
Gln Leu Phe Phe Thr Thr Pro Asp Leu Ser Al
#a Gln His Leu Gln Leu
                                85
# 90
# 95
Leu Thr Asp Asn Ser Pro Asp Gln Gly Pro As
#n Lys Val Phe Asp Leu
                        100
# 105
# 110
Cys Val Val Cys Gly Asp Lys Ala Ser Gly Ar
#g His Tyr Gly Ala Val
                115
# 120
# 125
Thr Cys Glu Gly Cys Lys Gly Phe Phe Lys Ar
#g Ser Ile Arg Lys Asn
        130
# 135
# 140
Leu Val Tyr Ser Cys Arg Gly Ser Lys Asp Cy
#s Ile Ile Asn Lys His
145 1
#50 1
#55 1
#60
His Arg Asn Arg Cys Gln Tyr Cys Arg Leu Gl
#n Arg Cys Ile Ala Phe
                                165
# 170
# 175
Gly Met Lys Gln Asp Ser Val Gln Cys Glu Ar
#g Lys Pro Ile Glu Val
                        180
# 185
# 190
Ser Arg Glu Lys Ser Ser Asn Cys Ala Ala Se
#r Thr Glu Lys Ile Tyr
                195
# 200
# 205
Ile Arg Lys Asp Leu Arg Ser Pro Leu Thr Al
#a Thr Pro Thr Phe Val
        210
# 215
# 220
Thr Asp Ser Glu Ser Thr Arg Ser Thr Gly Le
#u Leu Asp Ser Gly Met
225 2
#30 2
#35 2
#40
Phe Met Asn Ile His Pro Ser Gly Val Lys Th
#r Glu Ser Ala Val Leu
                                245
# 250
# 255
Met Thr Ser Asp Lys Ala Glu Ser Cys Gln Gl
#y Asp Leu Ser Thr Leu
                        260
# 265
# 270
Ala Asn Val Val Thr Ser Leu Ala Asn Leu Gl
#y Lys Thr Lys Asp Leu
                275
# 280
# 285
Ser Gln Asn Ser Asn Glu Met Ser Met Ile Gl
#u Ser Leu Ser Asn Asp
        290
# 295
# 300
Asp Thr Ser Leu Cys Glu Phe Gln Glu Met Gl
#n Thr Asn Gly Asp Val
305 3
#10 3
#15 3
#20
Ser Arg Ala Phe Asp Thr Leu Ala Lys Ala Le
#u Asn Pro Gly Glu Ser
                                325
# 330
# 335
Thr Ala Cys Gln Ser Ser Val Ala Gly Met Gl
#u Gly Ser Val His Leu
                        340
# 345
# 350
Ile Thr Gly Asp Ser Ser Ile Asn Tyr Thr Gl
#u Lys Glu Gly Pro Leu
                355
# 360
# 365
Leu Ser Asp Ser His Val Ala Phe Arg Leu Th
#r Met Pro Ser Pro Met
        370
# 375
# 380
Pro Glu Tyr Leu Asn Val His Tyr Ile Gly Gl
#u Ser Ala Ser Arg Leu
385 3
#90 3
#95 4
#00
Leu Phe Leu Ser Met His Trp Ala Leu Ser Il
#e Pro Ser Phe Gln Ala
                                405
# 410
# 415
Leu Gly Gln Glu Asn Ser Ile Ser Leu Val Ly
#s Ala Tyr Trp Asn Glu
                        420
# 425
# 430
Leu Phe Thr Leu Gly Leu Ala Gln Cys Trp Gl
#n Val Met Asn Val Ala
                435
# 440
# 445
Thr Ile Leu Ala Thr Phe Val Asn Cys Leu Hi
#s Asn Ser Leu Gln Gln
        450
# 455
# 460
Asp Ala Lys Val Ile Ala Ala Leu Ile His Ph
#e Thr Arg Arg Ala Ile
465 4
#70 4
#75 4
#80
Thr Asp Leu
<210> SEQ ID NO 16
<211> LENGTH: 2029
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 16
gaattcgggc ccgtcggctt tcttcaaccc tctcttcccg gagcgccccc aa
#tccacgag 60
tggcagccgc gggactgtcg cgtcggcgcc cgacgcggag tcagcagggg cg
#aaaagcgg 120
tagatcatgg caaccataga agaaattgca catcaaatta ttgaacaaca ga
#tgggagag 180
attgttacag agcagcaaac tgggcagaaa atccagattg tgacagcact tg
#atcataat 240
acccaaggca agcagttcat tctgacaaat cacgacggct ctactccaag ca
#aagtcatt 300
ctggccaggc aagattccac tccgggaaaa gttttcctta caactccaga tg
#cagcaggt 360
gtcaaccagt tattttttac cactcctgat ctgtctgcac aacacctgca gc
#tcctaaca 420
gataattctc cagaccaagg accaaataag gtttttgatc tttgcgtagt at
#gtggagac 480
aaagcatcag gacgtcatta tggagcagta acttgtgaag gctgcaaagg at
#tttttaaa 540
agaagcatcc gaaaaaattt agtatattca tgtcgaggat caaaggattg ta
#ttattaat 600
aagcaccacc gaaaccgctg tcaatactgc aggttacaga gatgtattgc gt
#ttggaatg 660
aagcaagact ctgtccaatg tgaaagaaaa cccattgaag tatcacgaga aa
#aatcttcc 720
aactgtgccg cttcaacaga aaaaatctat atccgaaagg accttcgtag cc
#cattaact 780
gcaactccaa cttttgtaac agatagtgaa agtacaaggt caacaggact gt
#tagattca 840
ggaatgttca tgaatattca tccatctgga gtaaaaactg agtcagctgt gc
#tgatgaca 900
tcagataagg ctgaatcatg tcagggagat ttaagtacat tggccaatgt gg
#ttacatca 960
ttagcgaatc ttggaaaaac taaagatctt tctcaaaata gtaatgaaat gt
#ctatgatt 1020
gaaagcttaa gcaatgatga tacctctttg tgtgaatttc aagaaatgca ga
#ccaacggt 1080
gatgtttcaa gggcatttga cactcttgca aaagcattga atcctggaga ga
#gcacagcc 1140
tgccagagct cagtagcggg catggaagga agtgtacacc taatcactgg ag
#attcaagc 1200
ataaattaca ccgaaaaaga ggggccactt ctcagcgatt cacatgtagc tt
#tcaggctc 1260
accatgcctt ctcctatgcc tgagtacctg aatgtgcact acattgggga gt
#ctgcctcc 1320
agactgctgt tcttatcaat gcactgggca ctttcgattc cttctttcca gg
#ctctaggg 1380
caagaaaaca gcatatcact ggtgaaagct tactggaatg aactttttac tc
#ttggtctt 1440
gcccagtgct ggcaagtgat gaatgtagca actatattag caacatttgt ca
#attgtctt 1500
cacaatagtc ttcaacaaga tgccaaggta attgcagccc tcattcattt ca
#caagacga 1560
gcaatcactg atttataaat gcttaactat agaatggctt atgactaccc aa
#aacagtgc 1620
cccatcaaca aatggggaaa attgcctttt gagctcagga ataatttata aa
#ttggggac 1680
taccttttag ttctttagca tattctattt cttattgttt tatataattt tt
#aaatcatt 1740
tgcttcctcc ttatgtttaa cagcagaggg gtaatcacct taaaatgtca tc
#aaaaatag 1800
atctactaga aggcagcatc acattcccat cttacttatg gactcctacc cc
#tggttcat 1860
gtcttatatg cctgtaatgg ttataaagcc taccttcagg aaagctatgg tt
#gactaatt 1920
actaatggat gggttttaaa catgtccctc tacaataaat taaaatcttt ca
#atgtttga 1980
atataatgtg gaggtgttta cctgagggcc tctctatctc cccgaattc
# 2029
<210> SEQ ID NO 17
<211> LENGTH: 596
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 17
Met Thr Ser Pro Ser Pro Arg Ile Gln Ile Il
#e Ser Thr Asp Ser Ala
  1 5
# 10
# 15
Val Ala Ser Pro Gln Arg Ile Gln Ile Val Th
#r Asp Gln Gln Thr Gly
                        20
# 25
# 30
Gln Lys Ile Gln Ile Val Thr Ala Val Asp Al
#a Ser Gly Ser Pro Lys
                35
# 40
# 45
Gln Gln Phe Ile Leu Thr Ser Pro Asp Gly Al
#a Gly Thr Gly Lys Val
        50
# 55
# 60
Ile Leu Ala Ser Pro Glu Thr Ser Ser Ala Ly
#s Gln Leu Ile Phe Thr
65
#70
#75
#80
Thr Ser Asp Asn Leu Val Pro Gly Arg Ile Gl
#n Ile Val Thr Asp Ser
                                85
# 90
# 95
Ala Ser Val Glu Arg Leu Leu Gly Lys Thr As
#p Val Gln Arg Pro Gln
                        100
# 105
# 110
Val Val Glu Tyr Cys Val Val Cys Gly Asp Ly
#s Ala Ser Gly Arg His
                115
# 120
# 125
Tyr Gly Ala Val Ser Cys Glu Gly Cys Lys Gl
#y Phe Phe Lys Arg Ser
        130
# 135
# 140
Val Arg Lys Asn Leu Thr Tyr Ser Cys Arg Se
#r Asn Gln Asp Cys Ile
145 1
#50 1
#55 1
#60
Ile Asn Lys His His Arg Asn Arg Cys Gln Ph
#e Cys Arg Leu Lys Lys
                                165
# 170
# 175
Cys Leu Glu Met Gly Met Lys Met Glu Ser Va
#l Gln Ser Glu Arg Lys
                        180
# 185
# 190
Pro Phe Asp Val Gln Arg Glu Lys Pro Ser As
#n Cys Ala Ala Ser Thr
                195
# 200
# 205
Glu Lys Ile Tyr Ile Arg Lys Asp Leu Arg Se
#r Pro Leu Ile Ala Thr
        210
# 215
# 220
Pro Thr Phe Val Ala Asp Lys Asp Gly Ala Ar
#g Gln Thr Gly Leu Leu
225 2
#30 2
#35 2
#40
Asp Pro Gly Met Leu Val Asn Ile Gln Gln Pr
#o Leu Ile Arg Glu Asp
                                245
# 250
# 255
Gly Thr Val Leu Leu Ala Thr Asp Ser Lys Al
#a Glu Thr Ser Gln Gly
                        260
# 265
# 270
Ala Leu Gly Thr Leu Ala Asn Val Val Thr Se
#r Leu Ala Asn Leu Ser
                275
# 280
# 285
Glu Ser Leu Asn Asn Gly Asp Thr Ser Glu Il
#e Gln Pro Glu Asp Gln
        290
# 295
# 300
Ser Ala Ser Glu Ile Thr Arg Ala Phe Asp Th
#r Leu Ala Lys Ala Leu
305 3
#10 3
#15 3
#20
Asn Thr Thr Asp Ser Ser Ser Ser Pro Ser Le
#u Ala Asp Gly Ile Asp
                                325
# 330
# 335
Thr Ser Gly Gly Gly Ser Ile His Val Ile Se
#r Arg Asp Gln Ser Thr
                        340
# 345
# 350
Pro Ile Ile Glu Val Glu Gly Pro Leu Leu Se
#r Asp Thr His Val Thr
                355
# 360
# 365
Phe Lys Leu Thr Met Pro Ser Pro Met Pro Gl
#u Tyr Leu Asn Val His
        370
# 375
# 380
Tyr Ile Cys Glu Ser Ala Ser Arg Leu Leu Ph
#e Leu Ser Met His Trp
385 3
#90 3
#95 4
#00
Ala Arg Ser Ile Pro Ala Phe Gln Ala Leu Gl
#y Gln Asp Cys Asn Thr
                                405
# 410
# 415
Ser Leu Val Arg Ala Cys Trp Asn Glu Leu Ph
#e Thr Leu Gly Leu Ala
                        420
# 425
# 430
Gln Cys Ala Gln Val Met Ser Leu Ser Thr Il
#e Leu Ala Ala Ile Val
                435
# 440
# 445
Asn His Leu Gln Asn Ser Ile Gln Glu Asp Ly
#s Leu Ser Gly Asp Arg
        450
# 455
# 460
Ile Lys Gln Val Met Glu His Ile Trp Lys Le
#u Gln Glu Phe Cys Asn
465 4
#70 4
#75 4
#80
Ser Met Ala Lys Leu Asp Ile Asp Gly Tyr Gl
#u Tyr Ala Tyr Leu Lys
                                485
# 490
# 495
Ala Ile Val Leu Phe Ser Pro Asp His Pro Gl
#y Leu Thr Ser Thr Ser
                        500
# 505
# 510
Gln Ile Glu Lys Phe Gln Glu Lys Ala Gln Me
#t Glu Leu Gln Asp Tyr
                515
# 520
# 525
Val Gln Lys Thr Tyr Ser Glu Asp Thr Tyr Ar
#g Leu Ala Arg Ile Leu
        530
# 535
# 540
Val Arg Leu Pro Ala Leu Arg Leu Met Ser Se
#r Asn Ile Thr Glu Glu
545 5
#50 5
#55 5
#60
Leu Phe Phe Thr Gly Leu Ile Gly Asn Val Se
#r Ile Asp Ser Ile Ile
                                565
# 570
# 575
Pro Tyr Ile Leu Lys Met Glu Thr Ala Glu Ty
#r Asn Gly Gln Ile Thr
                        580
# 585
# 590
Gly Ala Ser Leu
                595
<210> SEQ ID NO 18
<211> LENGTH: 596
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 18
Met Thr Ser Pro Ser Pro Arg Ile Gln Ile Il
#e Ser Thr Asp Ser Ala
  1 5
# 10
# 15
Val Arg Ser Pro Gln Arg Ile Gln Ile Val Th
#r Asp Gln Gln Thr Gly
                        20
# 25
# 30
Gln Lys Leu Gln Ile Val Thr Ala Val Asp Al
#a Ser Gly Ser Ser Lys
                35
# 40
# 45
Gln Gln Phe Ile Leu Thr Ser Pro Asp Gly Al
#a Gly Thr Gly Lys Val
        50
# 55
# 60
Ile Leu Ala Ser Pro Glu Thr Ser Ser Ala Ly
#s Gln Leu Ile Phe Thr
65
#70
#75
#80
Thr Ser Asp Asn Leu Val Pro Gly Arg Ile Gl
#n Ile Val Thr Asp Ser
                                85
# 90
# 95
Ala Ser Val Glu Arg Leu Leu Gly Lys Ala As
#p Val Gln Arg Pro Gln
                        100
# 105
# 110
Val Val Glu Tyr Cys Val Val Cys Gly Asp Ly
#s Ala Ser Gly Arg His
                115
# 120
# 125
Tyr Gly Ala Val Ser Cys Glu Gly Cys Lys Gl
#y Phe Phe Lys Arg Ser
        130
# 135
# 140
Val Arg Lys Asn Leu Thr Tyr Ser Cys Arg Se
#r Ser Gln Asp Cys Ile
145 1
#50 1
#55 1
#60
Ile Asn Lys His His Arg Asn Arg Cys Gln Ph
#e Cys Arg Leu Lys Lys
                                165
# 170
# 175
Cys Leu Glu Met Gly Met Lys Met Glu Ser Va
#l Gln Ser Glu Arg Lys
                        180
# 185
# 190
Pro Phe Asp Val Gln Arg Glu Lys Pro Ser As
#n Cys Ala Ala Ser Thr
                195
# 200
# 205
Glu Lys Ile Tyr Ile Arg Lys Asp Leu Arg Se
#r Pro Leu Ile Ala Thr
        210
# 215
# 220
Pro Thr Phe Val Ala Asp Lys Asp Gly Ser Ar
#g Gln Thr Gly Leu Leu
225 2
#30 2
#35 2
#40
Asp Pro Gly Met Leu Val Asn Ile Gln Gln Pr
#o Leu Ile Arg Glu Asp
                                245
# 250
# 255
Gly Thr Val Leu Leu Ala Thr Asp Ser Lys Al
#a Glu Thr Ser Gln Gly
                        260
# 265
# 270
Ala Leu Gly Thr Leu Ala Asn Val Val Thr Se
#r Leu Ala Asn Leu Ser
                275
# 280
# 285
Glu Ser Leu Asn Asn Gly Asp Ala Ser Glu Me
#t Gln Pro Glu Asp Gln
        290
# 295
# 300
Ser Ala Ser Glu Ile Thr Arg Ala Phe Asp Th
#r Leu Ala Lys Ala Leu
305 3
#10 3
#15 3
#20
Asn Thr Thr Asp Ser Ala Ser Pro Pro Ser Le
#u Ala Asp Gly Ile Asp
                                325
# 330
# 335
Ala Ser Gly Gly Gly Ser Ile His Val Ile Se
#r Arg Asp Gln Ser Thr
                        340
# 345
# 350
Pro Ile Ile Glu Val Glu Gly Pro Leu Leu Se
#r Asp Thr His Val Thr
                355
# 360
# 365
Phe Lys Leu Thr Met Pro Ser Pro Met Pro Gl
#u Tyr Leu Asn Val His
        370
# 375
# 380
Tyr Ile Cys Glu Ser Ala Ser Arg Leu Leu Ph
#e Leu Ser Met His Trp
385 3
#90 3
#95 4
#00
Ala Arg Ser Ile Pro Ala Phe Gln Ala Leu Gl
#y Gln Asp Cys Asn Thr
                                405
# 410
# 415
Ser Leu Val Arg Ala Cys Trp Asn Glu Leu Ph
#e Thr Leu Gly Leu Ala
                        420
# 425
# 430
Gln Cys Ala Gln Val Met Ser Leu Ser Thr Il
#e Leu Ala Ala Ile Val
                435
# 440
# 445
Asn His Leu Gln Asn Ser Ile Gln Glu Asp Ly
#s Leu Ser Gly Asp Arg
        450
# 455
# 460
Ile Lys Gln Val Met Glu His Ile Trp Lys Le
#u Gln Glu Phe Cys Asn
465 4
#70 4
#75 4
#80
Ser Met Ala Lys Leu Asp Ile Asp Gly His Gl
#u Tyr Ala Tyr Leu Lys
                                485
# 490
# 495
Ala Ile Val Leu Phe Ser Pro Asp His Pro Gl
#y Leu Thr Gly Thr Ser
                        500
# 505
# 510
Gln Ile Glu Lys Phe Gln Glu Lys Ala Gln Me
#t Glu Leu Gln Asp Tyr
                515
# 520
# 525
Val Gln Lys Thr Tyr Ser Glu Asp Thr Tyr Ar
#g Leu Ala Arg Ile Leu
        530
# 535
# 540
Val Arg Leu Pro Ala Leu Arg Leu Met Ser Se
#r Asn Ile Thr Glu Glu
545 5
#50 5
#55 5
#60
Leu Phe Phe Thr Gly Leu Ile Gly Asn Val Se
#r Ile Asp Ser Ile Ile
                                565
# 570
# 575
Pro Tyr Ile Leu Lys Met Glu Thr Ala Glu Ty
#r Asn Gly Gln Ile Thr
                        580
# 585
# 590
Gly Ala Ser Leu
                595
<210> SEQ ID NO 19
<211> LENGTH: 596
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 19
Met Thr Ser Pro Ser Pro Arg Ile Gln Ile Il
#e Ser Thr Asp Ser Ala
  1 5
# 10
# 15
Val Arg Ser Pro Gln Arg Ile Gln Ile Val Th
#r Asp Gln Gln Thr Gly
                        20
# 25
# 30
Gln Lys Leu Gln Ile Val Thr Ala Val Asp Al
#a Ser Gly Ser Ser Lys
                35
# 40
# 45
Gln Gln Phe Ile Leu Thr Ser Pro Asp Gly Al
#a Gly Thr Gly Lys Val
        50
# 55
# 60
Ile Leu Ala Ser Pro Glu Thr Ser Ser Ala Ly
#s Gln Leu Ile Phe Thr
65
#70
#75
#80
Thr Ser Asp Asn Leu Val Pro Gly Arg Ile Gl
#n Ile Val Thr Asp Ser
                                85
# 90
# 95
Ala Ser Val Glu Arg Leu Leu Gly Lys Ala As
#p Val Gln Arg Pro Gln
                        100
# 105
# 110
Val Val Glu Tyr Cys Val Val Cys Gly Asp Ly
#s Ala Ser Gly Arg His
                115
# 120
# 125
Tyr Gly Ala Val Ser Cys Glu Gly Cys Lys Gl
#y Phe Phe Lys Arg Ser
        130
# 135
# 140
Val Arg Lys Asn Leu Thr Tyr Ser Cys Arg Se
#r Ser Gln Asp Cys Ile
145 1
#50 1
#55 1
#60
Ile Asn Lys His His Arg Asn Arg Cys Gln Ph
#e Cys Arg Leu Lys Lys
                                165
# 170
# 175
Cys Leu Glu Met Gly Met Lys Met Glu Ser Va
#l Gln Ser Glu Arg Lys
                        180
# 185
# 190
Pro Phe Asp Val Gln Arg Glu Lys Pro Ser As
#n Cys Ala Ala Ser Thr
                195
# 200
# 205
Glu Lys Ile Tyr Ile Arg Lys Asp Leu Arg Se
#r Pro Leu Ile Ala Thr
        210
# 215
# 220
Pro Thr Phe Val Ala Asp Lys Asp Gly Ser Ar
#g Gln Thr Gly Leu Leu
225 2
#30 2
#35 2
#40
Asp Pro Gly Met Leu Val Asn Ile Gln Gln Pr
#o Leu Ile Arg Glu Asp
                                245
# 250
# 255
Gly Thr Val Leu Leu Ala Thr Asp Ser Lys Al
#a Glu Thr Ser Gln Gly
                        260
# 265
# 270
Ala Leu Gly Thr Leu Ala Asn Val Val Thr Se
#r Leu Ala Asn Leu Ser
                275
# 280
# 285
Glu Ser Leu Asn Asn Gly Asp Ala Ser Glu Me
#t Gln Pro Glu Asp Gln
        290
# 295
# 300
Ser Ala Ser Glu Ile Thr Arg Ala Phe Asp Th
#r Leu Ala Lys Ala Leu
305 3
#10 3
#15 3
#20
Asn Thr Thr Asp Ser Ala Ser Pro Pro Ser Le
#u Ala Asp Gly Ile Asp
                                325
# 330
# 335
Ala Ser Gly Gly Gly Ser Ile His Val Ile Se
#r Arg Asp Gln Ser Thr
                        340
# 345
# 350
Pro Ile Ile Glu Val Glu Gly Pro Leu Leu Se
#r Asp Thr His Val Thr
                355
# 360
# 365
Phe Lys Leu Thr Met Pro Ser Pro Met Pro Gl
#u Tyr Leu Asn Val His
        370
# 375
# 380
Tyr Ile Cys Glu Ser Ala Ser Arg Leu Leu Ph
#e Leu Ser Met His Trp
385 3
#90 3
#95 4
#00
Ala Arg Ser Ile Pro Ala Phe Gln Ala Leu Gl
#y Gln Asp Cys Asn Thr
                                405
# 410
# 415
Ser Leu Val Arg Ala Cys Trp Asn Glu Leu Ph
#e Thr Leu Gly Leu Ala
                        420
# 425
# 430
Gln Cys Ala Gln Val Met Ser Leu Ser Thr Il
#e Leu Ala Ala Ile Val
                435
# 440
# 445
Asn His Leu Gln Asn Ser Ile Gln Glu Asp Ly
#s Leu Ser Gly Asp Arg
        450
# 455
# 460
Ile Lys Gln Val Met Glu His Ile Trp Lys Le
#u Gln Glu Phe Cys Asn
465 4
#70 4
#75 4
#80
Ser Met Ala Lys Leu Asp Ile Asp Gly His Gl
#u Tyr Ala Tyr Leu Lys
                                485
# 490
# 495
Ala Ile Val Leu Phe Ser Pro Asp His Pro Gl
#y Leu Thr Gly Thr Ser
                        500
# 505
# 510
Gln Ile Glu Lys Phe Gln Glu Lys Ala Gln Me
#t Glu Leu Gln Asp Tyr
                515
# 520
# 525
Val Gln Lys Thr Tyr Ser Glu Asp Thr Tyr Ar
#g Leu Ala Arg Ile Leu
        530
# 535
# 540
Val Arg Leu Pro Ala Leu Arg Leu Met Ser Se
#r Asn Ile Thr Glu Glu
545 5
#50 5
#55 5
#60
Leu Phe Phe Thr Gly Leu Ile Gly Asn Val Se
#r Ile Asp Ser Ile Ile
                                565
# 570
# 575
Pro Tyr Ile Leu Lys Met Glu Thr Ala Glu Ty
#r Asn Gly Gln Ile Thr
                        580
# 585
# 590
Gly Ala Ser Leu
                595
<210> SEQ ID NO 20
<211> LENGTH: 615
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 20
Met Thr Ser Pro Ser Pro Arg Ile Gln Ile Il
#e Ser Thr Asp Ser Ala
  1 5
# 10
# 15
Val Ala Ser Pro Gln Arg Ile Gln Gly Ser Gl
#u Pro Ala Ser Gly Pro
                        20
# 25
# 30
Leu Ser Val Phe Thr Ser Leu Asn Lys Glu Ly
#s Ile Val Thr Asp Gln
                35
# 40
# 45
Gln Thr Gly Gln Lys Ile Gln Ile Val Thr Al
#a Val Asp Ala Ser Gly
        50
# 55
# 60
Ser Pro Lys Gln Gln Phe Ile Leu Thr Ser Pr
#o Asp Gly Ala Gly Thr
65
#70
#75
#80
Gly Lys Val Ile Leu Ala Ser Pro Glu Thr Se
#r Ser Ala Lys Gln Leu
                                85
# 90
# 95
Ile Phe Thr Thr Ser Asp Asn Leu Val Pro Gl
#y Arg Ile Gln Ile Val
                        100
# 105
# 110
Thr Asp Ser Ala Ser Val Glu Arg Leu Leu Gl
#y Lys Thr Asp Val Gln
                115
# 120
# 125
Arg Pro Gln Val Val Glu Tyr Cys Val Val Cy
#s Gly Asp Lys Ala Ser
        130
# 135
# 140
Gly Arg His Tyr Gly Ala Val Ser Cys Glu Gl
#y Cys Lys Gly Phe Phe
145 1
#50 1
#55 1
#60
Lys Arg Ser Val Arg Lys Asn Leu Thr Tyr Se
#r Cys Arg Ser Ser Gln
                                165
# 170
# 175
Asp Cys Ile Ile Asn Lys His His Arg Asn Ar
#g Cys Gln Phe Cys Arg
                        180
# 185
# 190
Leu Lys Lys Cys Leu Glu Met Gly Met Lys Me
#t Glu Ser Val Gln Ser
                195
# 200
# 205
Glu Arg Lys Pro Phe Asp Val Gln Arg Glu Ly
#s Pro Ser Asn Cys Ala
        210
# 215
# 220
Ala Ser Thr Glu Lys Ile Tyr Ile Arg Lys As
#p Leu Arg Ser Pro Leu
225 2
#30 2
#35 2
#40
Ile Ala Thr Pro Thr Phe Val Ala Asp Lys As
#p Gly Ala Arg Gln Thr
                                245
# 250
# 255
Gly Leu Leu Asp Pro Gly Met Leu Val Asn Il
#e Gln Gln Pro Leu Ile
                        260
# 265
# 270
Arg Glu Asp Gly Thr Val Leu Leu Ala Thr As
#p Ser Lys Ala Glu Thr
                275
# 280
# 285
Ser Gln Gly Ala Leu Gly Thr Leu Ala Asn Va
#l Val Thr Ser Leu Ala
        290
# 295
# 300
Asn Leu Ser Glu Ser Leu Asn Asn Gly Asp Th
#r Ser Glu Ile Gln Pro
305 3
#10 3
#15 3
#20
Glu Asp Gln Ser Ala Ser Glu Ile Thr Arg Al
#a Phe Asp Thr Leu Ala
                                325
# 330
# 335
Lys Ala Leu Asn Thr Thr Asp Ser Ser Ser Se
#r Pro Ser Leu Ala Asp
                        340
# 345
# 350
Gly Ile Asp Thr Ser Gly Gly Gly Ser Ile Hi
#s Val Ile Ser Arg Asp
                355
# 360
# 365
Gln Ser Thr Pro Ile Ile Glu Val Glu Gly Pr
#o Leu Leu Ser Asp Thr
        370
# 375
# 380
His Val Thr Phe Lys Leu Thr Met Pro Ser Pr
#o Met Pro Glu Tyr Leu
385 3
#90 3
#95 4
#00
Asn Val His Tyr Ile Cys Glu Ser Ala Ser Ar
#g Leu Leu Phe Leu Ser
                                405
# 410
# 415
Met His Arg Ala Arg Ser Ile Pro Ala Phe Gl
#n Gly Leu Gly Gln Asp
                        420
# 425
# 430
Cys Asn Thr Ser Leu Val Arg Ala Cys Trp As
#n Glu Leu Phe Thr Leu
                435
# 440
# 445
Gly Leu Ala Gln Cys Ala Gln Val Met Ser Le
#u Ser Thr Ile Leu Ala
        450
# 455
# 460
Ala Ile Val Asn His Leu Gln Asn Ser Ile Gl
#n Glu Asp Lys Leu Ser
465 4
#70 4
#75 4
#80
Gly Asp Arg Ile Lys Gln Val Met Glu His Il
#e Trp Lys Leu Gln Glu
                                485
# 490
# 495
Phe Cys Asn Ser Met Ala Asn Trp Asp Ile As
#p Gly Tyr Glu Tyr Ala
                        500
# 505
# 510
Tyr Leu Lys Ala Ile Val Leu Phe Ser Pro As
#p His Pro Gly Leu Thr
                515
# 520
# 525
Ser Thr Ser Gln Ile Glu Lys Phe Gln Glu Ly
#s Ala Gln Met Glu Leu
        530
# 535
# 540
Gln Asp Tyr Val Gln Lys Thr Tyr Ser Glu As
#p Thr Tyr Arg Leu Ala
545 5
#50 5
#55 5
#60
Arg Ile Leu Val Arg Leu Pro Ala Leu Arg Le
#u Met Ser Ser Asn Ile
                                565
# 570
# 575
Thr Glu Glu Leu Phe Phe Thr Gly Leu Ile Gl
#y Asn Val Ser Ile Asp
                        580
# 585
# 590
Ser Ile Ile Pro Tyr Ile Leu Lys Met Glu Th
#r Ala Glu Tyr Asn Gly
                595
# 600
# 605
Gln Ile Thr Gly Val Ser Leu
        610
# 615
<210> SEQ ID NO 21
<211> LENGTH: 615
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 21
Met Thr Ser Pro Ser Pro Arg Ile Gln Ile Il
#e Ser Thr Asp Ser Ala
  1 5
# 10
# 15
Val Ala Ser Pro Gln Arg Ile Gln Gly Ser Gl
#u Pro Ala Ser Gly Pro
                        20
# 25
# 30
Leu Ser Val Phe Thr Ser Leu Asn Lys Glu Ly
#s Ile Val Thr Asp Gln
                35
# 40
# 45
Gln Thr Gly Gln Lys Ile Gln Ile Val Thr Al
#a Val Asp Ala Ser Gly
        50
# 55
# 60
Ser Pro Lys Gln Gln Phe Ile Leu Thr Ser Pr
#o Asp Gly Ala Gly Thr
65
#70
#75
#80
Gly Lys Val Ile Leu Ala Ser Pro Glu Thr Se
#r Ser Ala Lys Gln Leu
                                85
# 90
# 95
Ile Phe Thr Thr Ser Asp Asn Leu Val Pro Gl
#y Arg Ile Gln Ile Val
                        100
# 105
# 110
Thr Asp Ser Ala Ser Val Glu Arg Leu Leu Gl
#y Lys Thr Asp Val Gln
                115
# 120
# 125
Arg Pro Gln Val Val Glu Tyr Cys Val Val Cy
#s Gly Asp Lys Ala Ser
        130
# 135
# 140
Gly Arg His Tyr Gly Ala Val Ser Cys Glu Gl
#y Cys Lys Gly Phe Phe
145 1
#50 1
#55 1
#60
Lys Arg Ser Val Arg Lys Asn Leu Thr Tyr Se
#r Cys Arg Ser Ser Gln
                                165
# 170
# 175
Asp Cys Ile Ile Asn Lys His His Arg Asn Ar
#g Cys Gln Phe Cys Arg
                        180
# 185
# 190
Leu Lys Lys Cys Leu Glu Met Gly Met Lys Me
#t Glu Ser Val Gln Ser
                195
# 200
# 205
Glu Arg Lys Pro Phe Asp Val Gln Arg Glu Ly
#s Pro Ser Asn Cys Ala
        210
# 215
# 220
Ala Ser Thr Glu Lys Ile Tyr Ile Arg Lys As
#p Leu Arg Ser Pro Leu
225 2
#30 2
#35 2
#40
Ile Ala Thr Pro Thr Phe Val Ala Asp Lys As
#p Gly Ala Arg Gln Thr
                                245
# 250
# 255
Gly Leu Leu Asp Pro Gly Met Leu Val Asn Il
#e Gln Gln Pro Leu Ile
                        260
# 265
# 270
Arg Glu Asp Gly Thr Val Leu Leu Ala Thr As
#p Ser Lys Ala Glu Thr
                275
# 280
# 285
Ser Gln Gly Ala Leu Gly Thr Leu Ala Asn Va
#l Val Thr Ser Leu Ala
        290
# 295
# 300
Asn Leu Ser Glu Ser Leu Asn Asn Gly Asp Th
#r Ser Glu Ile Gln Pro
305 3
#10 3
#15 3
#20
Glu Asp Gln Ser Ala Ser Glu Ile Thr Arg Al
#a Phe Asp Thr Leu Ala
                                325
# 330
# 335
Lys Ala Leu Asn Thr Thr Asp Ser Ser Ser Se
#r Pro Ser Leu Ala Asp
                        340
# 345
# 350
Gly Ile Asp Thr Ser Gly Gly Gly Ser Ile Hi
#s Val Ile Ser Arg Asp
                355
# 360
# 365
Gln Ser Thr Pro Ile Ile Glu Val Glu Gly Pr
#o Leu Leu Ser Asp Thr
        370
# 375
# 380
His Val Thr Phe Lys Leu Thr Met Pro Ser Pr
#o Met Pro Glu Tyr Leu
385 3
#90 3
#95 4
#00
Asn Val His Tyr Ile Cys Glu Ser Ala Ser Ar
#g Leu Leu Phe Leu Ser
                                405
# 410
# 415
Met His Arg Ala Arg Ser Ile Pro Ala Phe Gl
#n Gly Leu Gly Gln Asp
                        420
# 425
# 430
Cys Asn Thr Ser Leu Val Arg Ala Cys Trp As
#n Glu Leu Phe Thr Leu
                435
# 440
# 445
Gly Leu Ala Gln Cys Ala Gln Val Met Ser Le
#u Ser Thr Ile Leu Ala
        450
# 455
# 460
Ala Ile Val Asn His Leu Gln Asn Ser Ile Gl
#n Glu Asp Lys Leu Ser
465 4
#70 4
#75 4
#80
Gly Asp Arg Ile Lys Gln Val Met Glu His Il
#e Trp Lys Leu Gln Glu
                                485
# 490
# 495
Phe Cys Asn Ser Met Ala Asn Trp Asp Ile As
#p Gly Tyr Glu Tyr Ala
                        500
# 505
# 510
Tyr Leu Lys Ala Ile Val Leu Phe Ser Pro As
#p His Pro Gly Leu Thr
                515
# 520
# 525
Ser Thr Ser Gln Ile Glu Lys Phe Gln Glu Ly
#s Ala Gln Met Glu Leu
        530
# 535
# 540
Gln Asp Tyr Val Gln Lys Thr Tyr Ser Glu As
#p Thr Tyr Arg Leu Ala
545 5
#50 5
#55 5
#60
Arg Ile Leu Val Arg Leu Pro Ala Leu Arg Le
#u Met Ser Ser Asn Ile
                                565
# 570
# 575
Thr Glu Glu Leu Phe Phe Thr Gly Leu Ile Gl
#y Asn Val Ser Ile Asp
                        580
# 585
# 590
Ser Ile Ile Pro Tyr Ile Leu Lys Met Glu Th
#r Ala Glu Tyr Asn Gly
                595
# 600
# 605
Gln Ile Thr Gly Val Ser Leu
        610
# 615
<210> SEQ ID NO 22
<211> LENGTH: 2254
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 22
gaattcgccg ctcccacctc ggcgtctcgt ctctcgcccg ctgccccgcg ag
#cccgcggc 60
ccccggctcc cgccatccgc cgacaccggg agcccgggct ccccgcgccc tg
#ccctccgc 120
gccgggggcc gcccgccgca gacacgggac ccgcttcgag gccgctttgg cg
#caaatcct 180
gaggtaacac gtacacagac ctctcggccg gaatctccag ggatgaccag cc
#cctcccca 240
cgcatccaga taatctccac cgactctgct gtagcctcac ctcagcgcat tc
#agggctct 300
gaacctgcct ctggcccatt gagtgttttc acatctttga acaaagagaa ga
#ttgtcaca 360
gaccagcaga caggacagaa aatccagata gtcaccgcag tggacgcctc cg
#gatccccc 420
aaacagcagt tcatcctgac cagcccagat ggagctggaa ctgggaaggt ga
#tcctggct 480
tccccagaga catccagcgc caagcaactc atattcacca cctcagacaa cc
#tcgtccct 540
ggcaggatcc agattgtcac ggattctgcc tctgtggagc gtttactggg ga
#agacggac 600
gtccagcggc cccaggtggt agagtactgt gtggtctgtg gcgacaaagc ct
#ccggccgt 660
cactatgggg ctgtcagttg tgaaggttgc aaaggtttct tcaaaaggag tg
#tgaggaaa 720
aatttgacct acagctgccg gagcagccaa gactgcatca tcaataaaca tc
#accggaac 780
cgctgtcagt tttgccggct gaaaaaatgc ttagagatgg gcatgaaaat gg
#aatctgtg 840
cagagtgaac ggaagccctt cgatgtgcaa cgggagaaac caagcaattg tg
#ctgcttca 900
actgagaaaa tctatatccg gaaagacctg agaagtcccc tgatagctac tc
#ccacgttt 960
gtggcagaca aagatggagc aagacaaaca ggtcttcttg atccagggat gc
#ttgtgaac 1020
atccagcagc ctttgatacg tgaggatggt acagttctcc tggccacgga tt
#ctaaggct 1080
gaaacaagcc agggagctct gggcacactg gcaaatgtag tgacctccct tg
#ccaaccta 1140
agtgaatctt tgaacaacgg tgacacttca gaaatccagc cagaggacca gt
#ctgcaagt 1200
gagataactc gggcatttga taccttagct aaagcactta ataccacaga ca
#gctcctct 1260
tctccaagct tggcagatgg gatagacacc agtggaggag ggagcatcca cg
#tcatcagc 1320
agagaccagt cgacacccat cattgaggtt gaaggccccc tcctttcaga ca
#cacacgtc 1380
acatttaagc taacaatgcc cagtccaatg ccagagtacc tcaacgtgca ct
#acatctgt 1440
gagtctgcat cccgtctgct tttcctctca atgcaccggg caaggtcaat cc
#cagccttt 1500
cagggacttg gacaggactg caacaccagc cttgtgcggg cctgctggaa tg
#agctcttc 1560
accctcggcc tggcccagtg tgcccaggtc atgagtctct ccaccatcct gg
#ctgccatt 1620
gtcaaccacc tgcagaacag catccaggaa gataaacttt ctggtgaccg ga
#taaagcaa 1680
gtcatggagc acatctggaa gctgcaggag ttctgtaaca gcatggcgaa ct
#gggatata 1740
gatggctatg agtatgcata ccttaaagct atagttctct ttagccccga tc
#atccaggt 1800
ttgaccagca caagccagat tgaaaaattc caagaaaagg cacagatgga gt
#tgcaggac 1860
tatgttcaga aaacctactc agaagacacc taccgattgg cccggatcct cg
#ttcgcctg 1920
ccggcactca ggctgatgag ctccaacata acagaagaac ttttttttac tg
#gtctcatt 1980
ggcaatgttt cgatagacag cataatcccc tacatcctca agatggagac ag
#cagagtat 2040
aatggccaga tcaccggagt cagtctatag cgcaaaccac acacctgcca ag
#gagcaaca 2100
gaatccttcc aggaccgttc acatacaaag aaaagtagtg gtattttggt at
#gtgcaaat 2160
atttccatat gttagccatt tcctgtctgg tttctcctta tctgttaatc cc
#agacaata 2220
gcaattaaaa gactagtagg gatcctttcc tgac
#
# 2254
<210> SEQ ID NO 23
<211> LENGTH: 627
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 23
tatatgtgtg tcagtgtgtt gtgttattta tatgttgttg tagtgtgtat at
#gtttcttg 60
tattgtgtat atgtgtatat gtttgtgtat atgtgtatgt tatgtatgtt at
#gttgttat 120
gtatgtttgt gtgtttagtg tgtgtatata tttgtggaat gtgtatgtat gt
#ttttgtgc 180
aataaacaat tattatgtgt gtcctgttac acccagtgac taagttgtgt tt
#tgcacgcg 240
ccgtttgtgt tgccttcata ttatattata tatatttgta atatacctat ac
#tatgttac 300
ccccccccac ttgcaaccgt tttcggttgc ccttacatac actttcctca aa
#tttgttat 360
aacgtgtttt gtactaatcc catatgttgt gtgccaaggt acatattgcc ct
#gccaagta 420
tcctgccaac aacacacctg gcgccagggc gcggtattgc atgactaatg ta
#caataaac 480
ctgtcggttt gtacaatgtt gtggattgca gccaaaggtt aaaagcattt tt
#ggcttcta 540
gctgaacatt tttgtaccct tagtatatta tgcacaatac ccacaaaatg ag
#taacctaa 600
ggtcacacac ctgcgaccgg tttcggt
#
# 627
<210> SEQ ID NO 24
<211> LENGTH: 707
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 24
tatatgtgta tatgtactgt tatatatatg tgtgtatgta ctgttatgta ta
#tgtgttta 60
tgtactgtta tatgtatgtg tgttgtatat atgtgtgtat atatgtgtat gt
#gtgtatat 120
gtatatgtat gtgttgtgta tatatatgtg tgtgtgttat gtgtgtaatg ta
#atttattt 180
gtgtaatgtg tatgtgtgtt tatgtgcaat aaacaattaa ctacattatt gt
#atatcttg 240
ttacaccctg tgactcagtg gctgttgcac gcgttttggt ttgcacgcgc ct
#tacacaca 300
taagtaatat acatgcacaa tatatatatt tttgtttcca ataatatatt tt
#tatatttg 360
caaccgtttt cggttgccct tggcatacac tttccaccaa tttgttacaa cg
#tgttgcct 420
gttaatccta tatattttgt gccaggtaca cattgccctg ccaagttgat tg
#ccaagtgc 480
atcatatcct gccaaccaca cacctggcgc cagggtgcgg tattgcctta ct
#catatgtt 540
tattgccact gcaataaacc tgtctttgtg ttatactttt atgcactgta gc
#caactctt 600
aaaagcattt ttggcttgta gcagaacatt tttttgctct tactgtttgg tg
#tacaataa 660
cataaaaatg agtaacctaa ggtcacacac ctgcgaccgg tttcggt
# 707
<210> SEQ ID NO 25
<211> LENGTH: 675
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 25
tatatgtgta tatgtactgt tatatatatg tgtgtatgta ctgttatgta ta
#tgtgtgta 60
tgtactgtta tatgtatgtg tgttgtatat atgtgtgtat atatgtgtct gt
#gtgtatat 120
gtatatgtat gtgttgtgta tatatatgtg tgtgtgtgtt ctgtgtgtaa tg
#taagttat 180
ttgtgtaatg tgtatgtgtg tttatgtgca ataaacaatt acctcttgtt ac
#accctgtg 240
actcagtggc tgttgcacgc gttttggttt gcacgcgcct tacacacata ag
#taatatac 300
atgcacaata tatatatttt tgtttaaaat actatacttt tatatttgca ac
#cgttttcg 360
gttgccctta gcatacactt tccaccaatt tgttacaacg tgtttcctct ta
#atcctata 420
tattttgtgc caggtacaca ttgccctgcc aagttgcttg ccaagtgcat ca
#tatcctgc 480
caaccacaca cctggcgcca gggtgcggta ttgccttact cataaacctg tc
#tttgtgtt 540
atacttttat gcactgtagc caactcttaa aagcattttt ggcttgtagc ag
#cacatttt 600
tttgctctta ctgtttggta tacaataaca taaaaatgag taacctaagg tc
#acacacct 660
gcgaccggtt tcggt
#
#
# 675
<210> SEQ ID NO 26
<211> LENGTH: 883
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 26
cgtaagctgt aagtattgta tgtatgttga attagtgttg tttgttgttt at
#atgtttgt 60
atgtgcttgt atgtgcttgt aaatattacg ttgtatgtgt gtttgtatgt at
#ggtatagt 120
aaacacgtgt gtatgtgttt ttaaatgctt gtgtaactat tgtgtcatgc aa
#cataaata 180
aacttattgt ttcatcacct actaattgtg ttgtggttat tcattgtata ta
#aactatat 240
ttgctacatt ttgtttttgt tttatatata ctatattttg tagcgcaccg gc
#cattttgt 300
agcttcaacc gaattcggtt tgcatgcttt ttggcacaaa ctgtattttt tt
#aaatagtt 360
ctatgtcagc aactatagtt taaacttgta cgtttcctgc ttgccatgcg tg
#ccaaatcc 420
ctgttttcct gacctgcact gcttgccaac cattccattg ttttttacac tg
#cactatgt 480
gcaactactg aatcactatg tacgttgtgt catataaaat aaattactat gc
#gccaacgc 540
cttaaatacc gctgttaggc acatattttt ggcttgtttt aactcaccta at
#tgcatgtt 600
tggcataagg tttaaacttt taggccaact aaatgtcacc ttagttcata ca
#tgaactgt 660
gtaaaggtta gtcatacatt gttcatttgt aaaactgcac atgggtgtgt gc
#aaaccgtt 720
ttgggttaca catttacaag gaacttatat aataatacta aactacaata at
#tcatgtat 780
aaaactaagg gcgtaaccga aatcggttga accgaaaccg gttagtataa aa
#gcagacat 840
tttatgcacc aaaagagaac tgcaatgttt caggacccac agg
#
#883
<210> SEQ ID NO 27
<211> LENGTH: 494
<212> TYPE: PRT
<213> ORGANISM: Artificial Sequence
<220> FEATURE:
<223> OTHER INFORMATION: Description of Artificial
#Sequence:/note =
            synthetic construct
<400> SEQUENCE: 27
Met Leu Leu Leu Gly Leu Leu Leu Leu Pro Le
#u Leu Ala Gly Ala Arg
  1 5
# 10
# 15
Leu Leu Trp Asn Trp Trp Lys Leu Arg Ser Le
#u His Leu Pro Pro Leu
                        20
# 25
# 30
Ala Pro Gly Phe Leu His Leu Leu Gln Pro As
#p Leu Pro Ile Tyr Leu
                35
# 40
# 45
Leu Gly Leu Thr Gln Lys Phe Gly Pro Ile Ty
#r Arg Leu His Leu Gly
        50
# 55
# 60
Leu Gln Asp Val Val Val Leu Asn Ser Lys Ar
#g Thr Ile Glu Glu Ala
65
#70
#75
#80
Met Val Lys Lys Trp Ala Asp Phe Ala Gly Ar
#g Pro Glu Pro Leu Thr
                                85
# 90
# 95
Tyr Lys Leu Val Ser Lys Asn Tyr Pro Asp Le
#u Ser Leu Gly Asp Tyr
                        100
# 105
# 110
Ser Leu Leu Trp Lys Ala His Lys Lys Leu Th
#r Arg Ser Ala Leu Leu
                115
# 120
# 125
Leu Gly Ile Arg Asp Ser Met Glu Pro Val Va
#l Glu Gln Leu Thr Gln
        130
# 135
# 140
Glu Phe Cys Glu Arg Met Arg Ala Gln Pro Gl
#y Thr Pro Val Ala Ile
145 1
#50 1
#55 1
#60
Glu Glu Glu Phe Ser Leu Leu Thr Cys Ser Il
#e Ile Cys Tyr Leu Thr
                                165
# 170
# 175
Phe Gly Asp Lys Ile Lys Asp Asp Asn Leu Me
#t Pro Ala Tyr Tyr Lys
                        180
# 185
# 190
Cys Ile Gln Glu Val Leu Lys Thr Trp Ser Hi
#s Trp Ser Ile Gln Ile
                195
# 200
# 205
Val Asp Val Ile Pro Phe Leu Arg Phe Phe Pr
#o Asn Pro Gly Leu Arg
        210
# 215
# 220
Arg Leu Lys Gln Ala Ile Glu Lys Arg Asp Hi
#s Ile Val Glu Met Gln
225 2
#30 2
#35 2
#40
Leu Arg Gln His Lys Glu Ser Leu Val Ala Gl
#y Gln Trp Arg Asp Met
                                245
# 250
# 255
Met Asp Tyr Met Leu Gln Gly Val Ala Gln Pr
#o Ser Met Glu Glu Gly
                        260
# 265
# 270
Ser Gly Gln Leu Leu Glu Gly His Val His Me
#t Ala Ala Val Asp Leu
                275
# 280
# 285
Leu Ile Gly Gly Thr Glu Thr Thr Ala Asn Th
#r Leu Ser Trp Ala Val
        290
# 295
# 300
Val Phe Leu Leu His His Pro Glu Ile Gln Gl
#n Arg Leu Gln Glu Glu
305 3
#10 3
#15 3
#20
Leu Asp His Glu Leu Gly Pro Gly Ala Ser Se
#r Ser Arg Val Pro Tyr
                                325
# 330
# 335
Lys Asp Arg Ala Arg Leu Pro Leu Leu Asn Al
#a Thr Ile Ala Glu Val
                        340
# 345
# 350
Leu Arg Leu Arg Pro Val Val Pro Leu Ala Le
#u Pro His Arg Thr Thr
                355
# 360
# 365
Arg Pro Ser Ser Ile Ser Gly Tyr Asp Ile Pr
#o Glu Gly Thr Val Ile
        370
# 375
# 380
Ile Pro Asn Leu Gln Gly Ala His Leu Asp Gl
#u Thr Val Trp Glu Arg
385 3
#90 3
#95 4
#00
Pro His Glu Phe Trp Pro Asp Arg Phe Leu Gl
#u Pro Gly Lys Asn Ser
                                405
# 410
# 415
Arg Ala Leu Ala Phe Gly Cys Gly Ala Pro Va
#l Cys Leu Gly Glu Pro
                        420
# 425
# 430
Leu Ala Arg Leu Asp Leu Phe Val Val Leu Th
#r Arg Leu Leu Gln Ala
                435
# 440
# 445
Phe Thr Leu Leu Pro Ser Gly Asp Ala Leu Pr
#o Ser Leu Gln Pro Leu
        450
# 455
# 460
Pro His Cys Ser Val Ile Leu Lys Met Gln Pr
#o Phe Gln Val Arg Leu
465 4
#70 4
#75 4
#80
Gln Pro Arg Gly Met Gly Ala His Ser Pro Gl
#y Gln Asn Gln
                                485
# 490
<210> SEQ ID NO 28
<211> LENGTH: 5141
<212> TYPE: DNA
<213> ORGANISM: Artificial Sequence
<220> FEATURE:
<223> OTHER INFORMATION: Description of Artificial
#Sequence:/note =
            synthetic construct
<400> SEQUENCE: 28
agatctttct cccagtagct gctcagcatg gtggtggcat aagcccattt tc
#cggagcca 60
gggattcagt tgcagcaaga catggcccgg tctgggaggt caaccatgaa ga
#aggcagta 120
gctgtcattg cccaacccca gaaatcccaa tcctgttttc tccctctcag tc
#ctgatcat 180
ggattcagca gcagcgaact cgccaatgta gtgggtggca cagccagggt ct
#tgactctg 240
gctctgcagt agcacagtct ggaaaagctc tgaggggaga gagaccccca ct
#ggtccgag 300
ggtctggcac agagccagaa atggggggga aggtatgagg ctgggtcgcc tc
#tgacctct 360
caggtaccat ccaggaggcc ctggcctctc actgaacccg gccactcctc tt
#tggcatgg 420
cctcttccca aatccccaaa ctgcctcctt acccacaaaa gtggtctctg ag
#tgtcagtc 480
cagtgggacc cccacccctt atggcttcag ttccccaaat agggctggac cc
#ttgatcct 540
gatccagctg tggctatcca gccccttcct ggggactttg gactttgagg gg
#ggcatgcc 600
acttgtgctg ggaatccata ctttccctgg ctggagtaga acctgtggac tg
#tagtcctg 660
agggcagtca tgttctgcct gtgcctggaa agacaagaaa cttgactgca ga
#gagaagaa 720
agaggagaga ggaacagagc gaggaaaccg cccgtctccg gggctttttc tg
#ttccctat 780
ccttgacttt ctaagaccag tggggtcccc tcctctgctt ctttttcctg ag
#ttctgtga 840
aattccccaa ttcttatttt ttatctcaaa ccagctcaag gtgggctgtt tt
#cctttcaa 900
ccaaagaaag gtgctcctgg tggctaaagg tacatattcg acagctagat tt
#ccaggctg 960
gaatcctgcc ctccacaaca tgcgaacaat acccgtgttg catatagagc at
#ggctgtga 1020
agagttgagt gagtgcccac aaagcactta gagcagtgtc tggtacatgc ta
#ttactccg 1080
cagcgggaaa ccacttcctc ctttgtcttc tgggcacttt tgtgagtgaa ag
#gaggcact 1140
aataacaatc acactgggat acctgtatat actggaatgc cccaggcaaa cc
#aggcttaa 1200
actgtattac tctatctgta gcttaaacta acaaacaacc cacacaaatc ac
#attttgtt 1260
cttcaggcga ttcaggaagg cctattaggc agggactgcc attttctctc tg
#agacaaac 1320
atcattccat aaactggccc acggtgggtg gcagagggag agggcccagg tg
#ggggcgga 1380
cactattgcc tgcacagtga tgtggaacca gaaagctgac tctggatgca gg
#aaaaaggt 1440
cagggttgca tttcccttcc ttgcttcttg atgggtgatc aatttttttg aa
#atacggac 1500
gtcccaaggc caatgagact ggtgtcattc cagaaaaggg ccactctgtg gg
#cgggtcgg 1560
tgggagggta cctgaaggtg gggtcaaggg aggccccaaa acagtctaca ca
#gcaggagg 1620
gatggctggg gctcttgagc tataagtggc acctcagggc cctgacgggc gt
#ctcgccat 1680
gctgctcctg ggcctgctgc tgctgcccct gctggctggc gcccgcctgc tg
#tggaactg 1740
gtggaagctc cggagcctcc acctcccgcc tcttgccccg ggcttcttgc ac
#ttgctgca 1800
gcccgacctc ccaatctatc tgcttggcct gactcagaaa ttcgggccca tc
#tacaggct 1860
ccaccttggg ctgcaaggtg agaggctgat ctcgctctgg ccctcaccat ag
#gagggggc 1920
ggaggtgacg gagagggtcc tctctccgct gacgctgctt tggctgtctc cc
#agatgtgg 1980
tggtgctgaa ctccaagagg accattgagg aagccatggt caaaaagtgg gc
#agactttg 2040
ctggcagacc tgagccactt acctgtaagg gctgggggca ttttttcttt ct
#taaacaaa 2100
ttttttttta agagatgggt tcttgctatg ttgcccaggc tggtcttaaa tt
#cctagtct 2160
caaatgatcc tcccacctca gcctcaagtg tgagccacct ttggggcatc cc
#caatccag 2220
gtccctggaa gctcttgggg ggcatatctg gtggggagaa agcaggggtt gg
#ggaggccg 2280
aagaaggtca ggccctcagc tgccttcatc agttcccacc ctccagcccc ca
#cctcctcc 2340
tgcagacaag ctggtgtcta agaactaccc ggacctgtcc ttgggagact ac
#tccctgct 2400
ctggaaagcc cacaagaagc tcacccgctc agccctgctg ctgggcatcc gt
#gactccat 2460
ggagccagtg gtggagcagc tgacccagga gttctgtgag gtaaggctgg gc
#tcctgagg 2520
ccacctcggg tcagcctcgc ctctcacagt agcccccgcc ctgccgctgc ac
#agcggcct 2580
gctgaactca cactgtttct ccacagcgca tgagagccca gcccggcacc cc
#tgtggcca 2640
ttgaggagga attctctctc ctcacctgca gcatcatctg ttacctcacc tt
#cggagaca 2700
agatcaaggt gcctcacagc ccctcaggcc cacccccagc ccctccctga gc
#ctctcctt 2760
gtcctgaact gaaagtactc cctccttttc tggcaggacg acaacttaat gc
#ctgcctat 2820
tacaaatgta tccaggaggt gttaaaaacc tggagccact ggtccatcca aa
#ttgtggac 2880
gtgattccct ttctcagggt gaggacctgg agcctagaca cccctgggtt gt
#aggggaga 2940
ggctggggtg gagggagagg ctccttccca cagctgcatt ctcatgcttc ct
#gccgcagt 3000
tcttccccaa tccaggtctc cggaggctga agcaggccat agagaagagg ga
#tcacatcg 3060
tggagatgca gctgaggcag cacaaggtgg ggactgtacg tggacggcct cc
#cctcggcc 3120
cacagccagt gatgctaccg gcctcagcat tgctatgagg cgggttcttt tg
#catacccc 3180
agttatgggc ctgttgccac tctgtactcc tctccccagg ccagccgctc ag
#cccgctcc 3240
tttcaccctc tgcaggagag cctcgtggca ggccagtgga gggacatgat gg
#actacatg 3300
ctccaagggg tggcgcagcc gagcatggaa gagggctctg gacagctcct gg
#aagggcac 3360
gtgcacatgg ctgcagtgga cctcctgatc ggtggcactg agaccacagc aa
#acaccctc 3420
tcctgggccg tggttttttt gcttcaccac cctgaggtgc gtcctgggga ca
#agcaaaag 3480
gctccttccc agcaacctgg ccagggcggt gggcaccctc actcagctct ga
#gcactgtg 3540
cggctggggc tgtgcttgcc tcaccggcac tcaggctcac tgggttgctg ag
#ggagcggc 3600
tggaggctgg gcagctgtgg gctgctgggg caggactcca cccgatcatt cc
#ccagattc 3660
agcagcgact gcaggaggag ctagaccacg aactgggccc tggtgcctcc ag
#ctcccggg 3720
tcccctacaa ggaccgtgca cggctgccct tgctcaatgc caccatcgcc ga
#ggtgctgc 3780
gcctgcggcc cgttgtgccc ttagccttgc cccaccgcac cacacggccc ag
#caggtgac 3840
tcccgagggt tggggatgag tgaggaaagc ccgagcccag ggaggtcctg gc
#cagcctct 3900
aactccagcc cccttcagca tctccggcta cgacatccct gagggcacag tc
#atcattcc 3960
gaacctccaa ggcgcccacc tggatgagac ggtctgggag aggccacatg ag
#ttctggcc 4020
tggtatgtgg gggccggggg cctgccgtca aaatgtggtg gaggctggtc cc
#cgctgccg 4080
ctgaacgcct ccccacccac ctgtccaccc gcccgcagat cgcttcctgg ag
#ccaggcaa 4140
gaactccaga gctctggcct tcggctgcgg tgccccggtg tgcctgggcg ag
#ccgctggc 4200
gcgcctggac ctcttcgtgg tgctgacccg actgctgcag gccttcacgc tg
#ctgccctc 4260
cggggacgcc ctgccctccc tgcagcccct gccccactgc agtgtcatcc tc
#aagatgca 4320
gcctttccaa gtgcggctgc agccccgggg gatgggggcc cacagcccag gc
#cagaacca 4380
gtgatggggc aggaccgatg ccagccgggt acctcagttt ctcctttatt gc
#tcccgtac 4440
gaacccctcc cctcccccct gtaaacacag tgctgcgaga tcgctggcag ag
#aaggcttc 4500
ctccagcggc tgggtggtga aggaccctgg ctcttctctc ggggcgaccc ct
#cagtgctc 4560
ggcagtcata ctggggtgcg agagaggtgg gcagcagctc agcctccccc cg
#ctggggag 4620
cgaaagtttc ttggtctcag cttcatttcc gtgaagggca ccgagaactc ga
#agcccttc 4680
cagtggtacc agctcactcc ctgggaaagg ggttgtcaag agagagtcaa ag
#ccggatgt 4740
cccatctgct cttcccgttc cccttaagga ggtagctccc agcactcaac ca
#acctcccc 4800
gcagagctcc cttcctgacc ctccgctgca gaggattgag gcttaattct ga
#gctggccc 4860
tttccagcca ataaatcaac tccagctccc tctgcgaggc tggcatgatt gt
#tccatttc 4920
acccagccgc tcagtccctt gcctgttaca ctgtggggct gaaacctagg ca
#ggccgagc 4980
cccagccacc ccagctctga gccggcctcc ccacccctca cctgatggtc ca
#ctgtgctc 5040
ccgtagagcc cgttgaggtt ggcgtagtgg cagttcctgt accaccaggc cc
#ctcggtag 5100
gagacagcgc aggagatgag caagctgttg gggtcccgat c
#
# 5141
```

1. A method of inhibiting HPV infection comprising interfering with TR2 or TR4 interaction with a TR2/TR4 response element in the HPV genome.

2. The method of claim 1, wherein the interfering occurs in stratified squamous epithelial cells.

3. The method of claim 1, wherein the interfering occurs in vaginal or cervix tissue.

4. The method of claim 1, wherein the interfering comprises the administration of a TR2 or TR4 molecule lacking a functional activation domain.

5. The method of claim 1 ,wherein the interfering comprises the administration of a TR2 or TR4 molecule lacking a functional ligand binding domain.

6. The method of claim 4, wherein the TR2 or TR4 consists of a DNA binding domain.

7. The method of claim 1, wherein the response element is a DR4 response element.

8. The method of claim 8, wherein the DR4 response element is located 175 bp upstream of the TATA-box at bp 65 of the E6 ORF and comprises SEQ ID No:1.

9. The method of claim 8, wherein the DR4 response element comprises the sequences set forth in SEQ ID NO:2.

10. The method of claim 8, wherein the DR4 response element comprises the sequence set forth in SEQ ID NO:3.

11. The method of claim 8, wherein the DR4 response element comprises the sequence set forth in SEQ ID NO:4.

12. A method of identifying an inhibitor of TR2 or TR4 HPV DR4 binding comprising forming an incubation mixture comprising a potential inhibitor, TR2, TR4, or combination thereof, and a nucleic acid comprising SEQ ID NO:1, SEQ ID NO:2, SEQ ID NO:3, or SEQ ID NO:4 in double stranded form, and assaying whether the potential inhibitor decreases the amount of TR2, TR4, or combination thereof bound to SEQ ID NO:1, SEQ ID NO:2, SEQ ID NO:3, or SEQ ID NO:4.

13. A method of producing an inhibitor of TR2 or TR4 HPV DR4 binding comprising identifying a potential inhibitor of TR2 or TR4 HPV DR4 binding comprising forming an incubation mixture comprising a potential inhibitor, TR2, TR4, or combination thereof, and a nucleic acid comprising SEQ ID NO:1, SEQ ID NO:2, SEQ ID NO:3, or SEQ ID NO:4 in double stranded form, and assaying whether the potential inhibitor decreases the amount of TR2, TR4, or combination thereof bound to SEQ ID NO:1, SEQ ID NO:2, SEQ ID NO:3, or SEQ ID NO:4.

14. A method of manufacturing an inhibitor of TR2 or TR4 HPV DR4 binding comprising synthesizing the potential inhibitor of claim 12.

15. A method of manufacturing a potential inhibitor of TR2 or TR4 HPV DR4 binding comprising admixing the potential inhibitor of claim 12 with a pharmaceutical carrier.

16. The method of claim 1, wherein the interfering occurs with the TR2 interaction with a TR2/TR4 response element in the HPV genome.

17. The method of claim 1, wherein the interfering occurs with the TR4 interaction with a TR2/TR4 response element in the HPV genome.

18. A method of inhibiting 21-Ohase gene expression comprising administering TR4 protein.

19. A method of inhibiting 21 -Ohase gene expression comprising administering TR4 protein comprising a sequence having at least 68% identity to SEQ ID NO:17.

20. The method of claim 19, wherein any difference between the TR4 protein sequence and SEQ ID NO:17 is a conserved substitution.

21. The method of claim 18 wherein the binding of TR4 protein to a nucleic acid molecule comprising SEQ ID NO:6 AGGTCA is reduced.

22. The method of claim 21, wherein the NBRE is located at −228 of the 21-Ohase gene set forth in SEQ ID NO:7.

23. The method of claim 22, wherein the 21-Ohase gene expression occurs in a human cell.

24. A method of inhibiting transcription of a gene comprising administering TR4, wherein TR4 binds a monomeric binding site associated with the gene.

25. The method of claim 24, wherein the gene is the 21-Ohase gene.

26. A method of identifying an inhibitor of 21-Ohase expression comprising forming an incubation mixture comprising a potential inhibitor and a nucleic acid comprising SEQ ID NO:6 in double stranded form, and assaying whether the potential inhibitor decreases the amount of TR2, TR4, or combination thereof bound to SEQ ID NO:6, and selecting those potential inhibitors that bind SEQ ID NO:6.

27. A method of producing an inhibitor of 21-Ohase gene expression, comprising identifying the inhibitor of 21-Ohase expression comprising forming an incubation mixture comprising a potential inhibitor and a nucleic acid comprising SEQ ID NO:1 or SEQ ID NO:2 in double stranded form, and assaying whether the potential inhibitor decreases the amount of TR2, TR4, or combination thereof bound to SEQ ID NO:1 or SEQ ID NO:2, and selecting those potential inhibitors that bind SEQ ID NO:1 or SEQ ID NO:2.

28. A method of manufacturing an inhibitor of 21-Ohase gene expression comprising synthesizing the potential inhibitor of claim 27.

29. A method of manufacturing a potential inhibitor 21-Ohase comprising admixing the potential inhibitor of claim 26 with a pharmaceutical carrier.

30. A composition comprising 1) an antisense TR4 and 2) a repressor for the expression of the antisense TR4.

31. The method of claim 30, wherein the expression of the repressor is inducible.

32. The method of claim 31, wherein the repressor is a Tet repressor.

33. A system comprising 1) a nucleic acid, wherein the nucleic acid expresses an antisense TR4 under the control of a repressor, and 2) the nucleic acid expresses the repressor.

34. A system comprising 1) a first nucleic acid, wherein the nucleic acid expresses an antisense TR4 under the control of a repressor, and 2) a second nucleic acid, wherein the second nucleic acid expresses the repressor.

35. The system of claim 33, wherein the repressor is a Tetr-Krab repressor.

36. The system of claim 33 wherein the repressor is under the control of a constitutive promoter.

37. The system of claim 33 wherein the promoter is a CMV promoter.

38. The system of claim 33 wherein the system is a cell.

39. The system of claim 38, wherein the cell is a human cell.

40. A method of inhibiting RA induced cellular activity comprising administering a composition that inhibits TR4 function.

41. The method of claim 40, wherein the composition is an antisense nucleic acid.

42. The method of claim 40, wherein the interfering comprises the administration of a TR4 molecule lacking a functional activation domain.

43. The method of claim 1, wherein the interfering comprises the administration of a TR4 molecule lacking a functional ligand binding domain.

44. The method of claim 42 wherein the TR4 consists of a DNA binding domain.

45. The method of claim 40, wherein the cellular activity is differentiation, cell cycle progression, cell morphology, or cell apoptosis.

46. A system for testing the effect of compounds on RA induced cellular activity comprising a cell expressing an inhibitor of T4, T2, or a combination thereof.

47. The system of claim 46, wherein the inhibitor is controlled by an inducible system.

48. The system of claim 47, wherein the inducible system is a tetracycline inducible system.

49. The system of claim 46, wherein the inducible system comprises a first plasmid and a second plasmid.

50. The method of claim 49, wherein the first plasmid comprises a transcriptional silencer protein under the control of a constitutive promoter.

51. The method of claim 50, wherein the first plasmid further comprises sequence encoding a poly A tail or a nuclear localization signal.

52. The method of claim 50, wherein the promoter is a CMV promoter.

53. The method of claim 50 wherein the transcriptional; silencer protein is TetR-KRAB.

54. The method of claim 49 wherein the second plasmid comprises a TR4 antisense molecule a TetR-KRAB site, and a strong constitutive promoter.

55. The method of claim 54, wherein there are 7 TetR-KRAB sites.

56. The method of claim 54, wherein the promoter is the CMV promoter.

57. The method of claim 50 wherein the first and second plasmid further comprise a selection marker.

58. A composition for modulating apoptosis, wherein the composition inhibits the expression of TR4.

59. The composition of claim 58, wherein the composition is an antisense molecule of TR4

60. A method of modulating retinoic acid induced apoptosis, comprising administering a modulator of TR4 activity.

61. The method of claim 60, wherein the modulator inhibits the RA induced apoptotic activity.

62. The method of claim 60 wherein the inhibitor reduces the expression of TR4.

63. The method of claim 62, wherein the inhibitor is an antisense molecule that interacts with TR4 mRNA.

64. A method of reducing RA mediated transactivation in a cell comprising administering TP2 to the cell.

65. The method of claim 64, wherein the cell is a human HaCaT Keratinocyte.

66. The method of claim 64, wherein the cell is a CV1 cell.

67. The method of claim 64, wherein the RA mediated transactivation is reduced by at least 44%

68. A method of reducing RA mediated transactivation in a cell comprising administering TR4 to the cell.

69. The method of claim 68, wherein the cell is a human HaCaT Keratinocyte.

70. The method of claim 69, wherein the RA mediated transactivation is reduced by at least 47%.

71. The method of claim 64, wherein the interfering comprises the administration of a TR2 molecule lacking a functional activation domain.

72. The method of claim 64, wherein the interfering comprises the administration of a TR2 molecule lacking a functional ligand binding domain.

73. The method of claim 71 wherein the TR2 consists of a DNA binding domain.

74. A method of inhibiting RA mediated transactivation comprising administering TR2 protein.

75. A method of inhibiting RA mediated transactivation comprising administering TR2 protein comprising a sequence having at least 68% identity to SEQ ID NO:6, 7, 8, 9, 11, 13, or 15.

76. The method of claim 75, wherein any difference between the TR4 protein sequence and SEQ ID NO: 6, 7, 8, 9, 11, 13, or 15 is a conserved substitution.

77. A method of regulating transactivation activity of the Wy-14643-mediated peroxisome proliferator-activated receptor α (PPARα) comprising administering TR4.

78. The method of claim 77, wherein the interfering comprises the administration of a TR4 molecule lacking a functional activation domain.

79. The method of claim 77, wherein the interfering comprises the administration of a TR4 molecule lacking a functional ligand binding domain.

80. The method of claim 78 wherein the TR4 consists of a DNA binding domain.

81. A method of regulating transactivation activity of the Wy-14643-mediated peroxisome proliferator-activated receptor α (PPARα) comprising administering TR4 protein comprising a sequence having at least 68% identity to SEQ ID NO:17.

82. The method of claim 81, wherein any difference between the TR4 protein sequence and SEQ ID NO:17 is a conserved substitution.

83. A method of regulating the expression of peroxisome proliferator-activated receptor α (PPARα) comprising contacting the PPRE of peroxisome proliferator-activated receptor α (PPARα) with TR2.

84. The method of claim 83, wherein the interfering comprises the administration of a TR2 molecule lacking a functional activation domain.

85. The method of claim 83, wherein the interfering comprises the administration of a TR2 molecule lacking a functional ligand binding domain.

86. The method of claim 84 wherein the TR2 consists of a DNA binding domain.

87. A method of regulating the expression of peroxisome proliferator-activated receptor α (PPARα) comprising contacting the PPRE of peroxisome proliferator-activated receptor α (PPARα) with a TR2 protein comprising a sequence having at least 68% identity to SEQ ID NO:6, 7, 8, 9, 11, 13, or 15.

88. The method of claim 87, wherein any difference between the TR4 protein sequence and SEQ ID NO: 6, 7, 8, 9, 11, 13, or 15 is a conserved substitution.

89. A method of regulating the expression of peroxisome proliferator-activated receptor α (PPARα) comprising contacting the PPRE of peroxisome proliferator-activated receptor α (PPARα) with TR4.

90. The method of claim 89, wherein the interfering comprises the administration of a TR4 molecule lacking a functional activation domain.

91. The method of claim 89, wherein the interfering comprises the administration of a TR4 molecule lacking a functional ligand binding domain.

92. The method of claim 90 wherein the TR4 consists of a DNA binding domain.

93. A method of regulating the expression of peroxisome proliferator-activated receptor α (PPARα) comprising contacting the PPRE of peroxisome proliferator-activated receptor α (PPARα) with a TR4 comprising a sequence having at least 68% identity to SEQ ID NO:17.

94. The method of claim 93, wherein any difference between the TR4 protein sequence and SEQ ID NO:17 is a conserved substitution.

95. The method of claim 94, wherein the PPRE has the sequence set forth in SEQ ID NO:5.

96. A method of regulating the expression of TR2 comprising administering Wy-14643.

97. A method of regulating the expression of TR4 comprising administering Wy-14643.

98. A composition comprising 1) an antisense TR2 and 2) a repressor for the expression of the antisense TR2.

99. The method of claim 98, wherein the expression of the repressor is inducible.

100. The method of claim 99, wherein the repressor is a Tet repressor.

101. A system comprising 1) a nucleic acid, wherein the nucleic acid expresses an antisense TR2 under the control of a repressor, and 2) the nucleic acid expresses the repressor.

102. A system comprising 1) a first nucleic acid, wherein the nucleic acid expresses an antisense TR2 under the control of a repressor, and 2) a second nucleic acid, wherein the second nucleic acid expresses the repressor.

103. The system of claim 101 wherein the repressor is a Tetr-Krab repressor.

104. The system of claim 101 wherein the repressor is under the control of a constitutive promoter.

105. The system of claim 101 wherein the promoter is a CMV promoter.

106. The system of claim 101 wherein the system is a cell.

107. The system of claim 105, wherein the cell is a human cell.
